(19) United States
(12) Patent Application Publication
(10) Pub. No.: US 2022/0274965 A1
(43) Pub. Date: Sep. 1, 2022

(54) ANTIVIRAL AGENTS AND USES THEREOF

(71) Applicant: GRIFFITH UNIVERSITY, Nathan (AU)

(72) Inventors: Mark Von Itzstein, Palm Beach (AU); Ibrahim El-Deeb, Runaway Bay (AU); Larissa Heilig, Burleigh Waters (AU); Patrice Guillon, Southport (AU)

(73) Assignee: GRIFFITH UNIVERSITY, Nathan (AU)

(21) Appl. No.: 17/631,257

(22) PCT Filed: Jul. 30, 2020
(86) PCT No.: PCT/AU2020/050784

§ 371 Date: Jan. 28, 2022

(30) Foreign Application Priority Data
Jul. 30, 2019 (AU) 2019902717

(51) Int. Cl. C07D 405/04; C07D 405/14; C07D 471/04; C07D 491/056; C07D 495/04

(52) U.S. Cl. CPC C07D 405/04; C07D 405/14; C07D 471/04; C07D 491/056; C07D 495/04

(57) ABSTRACT

The present invention relates to a compound of formula (I), or a pharmaceutically acceptable salt thereof: Formula (I) In which R₃ is selected from the group consisting of optionally substituted N-linked naphthotriazole, optionally substituted N-linked indazole, and certain N-linked triazoles. The present invention also relates to uses of the compounds in treating a disease, disorder or condition caused by viral infection, and pharmaceutical compositions comprising the compounds.

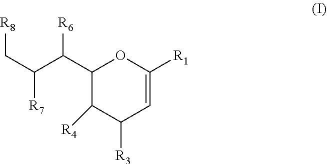

### FIELD OF THE INVENTION

The invention relates to the field of medical treatment. More particularly, this invention relates to novel antiviral agents and their use in treating a disease or condition caused by a viral infection.

### BACKGROUND TO THE INVENTION

Any reference to background art herein is not to be construed as an admission that such art constitutes common general knowledge in Australia or elsewhere.

Viruses are responsible for a wide range of mammalian disease which represents a great cost to society. The effects of viral infection can range from common flu symptoms to serious respiratory problems and can result in death, particularly amongst the young, elderly and immunocompromised members of the community.

Viruses of the family Orthomyxoviridae, including influenza virus types A, B and C, and the family Paramyxoviridae are the pathogenic organisms responsible for a significant number of human infections annually.

Taking the family Paramyxoviridae as one example, human parainfluenza viruses types 1 and 3 (hPIV-1 and 3) are a leading cause of upper and lower respiratory tract disease in infants and young children and impact the elderly and immunocompromised. Significantly, it is estimated that in the United States alone up to five million lower respiratory tract infections occur each year in children under 5 years old, and hPIV has been isolated in approximately one third of these cases. hPIV infections are frequently reported in transplant patients, with the mortality rate as high as 30% in hematopoietic stem cell transplant patients. There are currently neither vaccines nor specific antiviral therapy to prevent or treat hPIV infections respectively, despite continuing efforts. Some of the more recent approaches have focused on an entry blockade and the triggering of premature virus fusion by a small molecule.

An initial interaction of the parainfluenza virus with the host cell is through its surface glycoprotein, haemagglutinin-neuraminidase (HN) and involves recognition of N-acetylneuraminic acid-containing glycoconjugates. The parainfluenza virus HN is a multifunctional protein that encompasses the functions of receptor binding (for cell adhesion) and receptor destruction (facilitating virus release), not only within the one protein, but apparently in a single binding site. In addition, the HN is involved in activation of the viral surface fusion (F) protein necessary to initiate infection of the target host cell. Inhibition of haemagglutinin-neuraminidase may therefore provide a target for antivirals.

Certain antiviral compounds have been disclosed in the present Applicant's earlier filed International Application published as WO 2016/033660 as modulators of viral haemagglutinin-neuraminidase functions. While suitable for their purpose, the publication provides limited guidance in terms of the variability which is tolerated at certain key positions and optimal substitutions for efficacy.

### SUMMARY OF INVENTION

According to a first aspect of the invention, there is provided a compound of formula (I), or a pharmaceutically acceptable salt thereof:

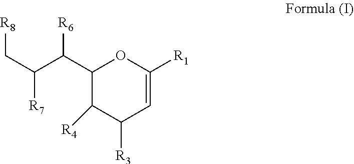

wherein, R₁ is selected from the group consisting of COOH, or a salt thereof, C(O)NR₉R₁₀, C(O)OR₁₁ wherein R₉, R₁₀ and R₁₁ are independently selected from the group consisting of hydrogen, optionally substituted C₁-C₆ alkyl and optionally substituted aryl;

R₃ is selected from the group consisting of optionally substituted N-linked naphthotriazole, optionally substituted N-linked indazole, and N-linked triazole of the following formula:

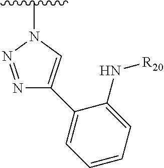

wherein R₂₀ is selected from the group consisting of

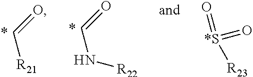

wherein, * is the point of attachment, and R₂₁, R₂₂ and R₂₃ are independently selected from the group consisting of optionally substituted alkyl, optionally substituted aryl, optionally substituted alkylheterocyclic, optionally substituted alkylheteroaryl, optionally substituted alkylamine, optionally substituted dialkylamine and an optionally substituted linker which links the compound to another compound of Formula (I);

R₄ is selected from the group consisting of sulfonamide, urea and NHC(O)R₁₇ wherein R₁₇ is selected from the group consisting of C₁-C₆ alkyl, C₁-C₆ haloalkyl and C₃-C₆ cycloalkyl, all of which may be optionally substituted;

R₆, R₇ and R₈ are independently selected from the group consisting of OH, protected OH, NH₂, C₁-C₆ alkyl, C₁-C₆ haloalkyl, NR₁₈R₁₈′, C₁-C₆ alkoxy, C₁-C₆ haloalkoxy, —OC(O)R₁₈, —NH(C═O)R₁₈, and S(O)ₙR₁₈, wherein n=0-2 and each R₁₈ and R₁₈′ are independently selected from hydrogen, optionally substituted C₁-C₆ alkyl and optionally substituted C₁-C₉ alkanoyl, as appropriate.

In one embodiment of the first aspect, the compound of formula (I) is a compound of formula (II):

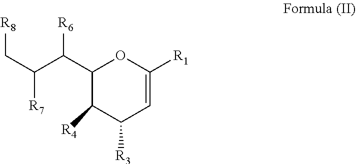

wherein, R₁, R₃, R₄, R₆, R₇ and R₈ are as described above.

According to a second aspect of the invention there is provided a pharmaceutical composition comprising an effective amount of a compound of the first aspect, or a pharmaceutically acceptable salt thereof, and a pharmaceutically acceptable carrier, diluent and/or excipient.

Suitably, the pharmaceutical composition is for the treatment or prophylaxis of a disease, disorder or condition caused by viral infection.

A third aspect of the invention resides in a method of treating a disease, disorder or condition caused by viral infection in a patient including the step of administering an effective amount of a compound of the first aspect, or a pharmaceutically effective salt thereof, or the pharmaceutical composition of the second aspect to the patient.

A fourth aspect of the invention provides for a compound of the first aspect, or a pharmaceutically effective salt thereof, or the pharmaceutical composition of the second aspect for use in the treatment of a disease, disorder or condition caused by viral infection in a patient.

A fifth aspect of the invention provides for use of a compound of the first aspect, or a pharmaceutically effective salt thereof, in the manufacture of a medicament for the treatment of a disease, disorder or condition caused by viral infection.

In one embodiment of the third, fourth or fifth aspects, the disease, disorder or condition is selected from parainfluenza, influenza, croup, bronchiolitis and pneumonia.

In one embodiment of the third, fourth or fifth aspects, the disease, disorder or condition is parainfluenza and/or influenza.

In embodiments, the viral respiratory infection may be caused by one or more of an influenza A virus, influenza B virus, influenza C virus, influenza D virus, parainfluenza virus, respiratory syncytial virus (RSV) and human metapneumovirus (hMPV).

When the disease, disorder or condition is influenza then it may be influenza A, B, C or D.

When the disease, disorder or condition is parainfluenza viral infection, it may be selected from the group consisting of an hPIV-1, -2, -3 and -4 virus. These may include all viral subtypes, e.g. 4a and 4b.

When the disease, disorder or condition is caused by RSV then it may be the A and/or B subtypes, for example, hRSV-A and hRSV-B.

When the disease, disorder or condition is caused by hMPV then it may be caused by any one or more of the hMPV A1, A2, B1 and B2 subtypes.

Preferably, the patient is a domestic or livestock animal or a human.

A sixth aspect of the invention provides for a method of modulating viral haemagglutinin and/or neuraminidase function including the step of contacting the viral haemagglutinin-neuraminidase with a compound of the first aspect.

The various features and embodiments of the present invention, referred to in individual sections above apply, as appropriate, to other sections, mutatis mutandis. Consequently, features specified in one section may be combined with features specified in other sections as appropriate.

Further features and advantages of the present invention will become apparent from the following detailed description.

### DETAILED DESCRIPTION

The present invention is predicated, at least in part, on the finding that certain neuraminic acid derivatives with modifications at key positions, including the C-4 position of the ring, display useful efficacy in the treatment of diseases caused by viral respiratory infection. Particularly, the compounds of the invention are useful in the inhibition of parainfluenza haemagglutinin-neuraminidase functions. This may be considered in terms of blocking the haemagglutination function and/or the neuraminidase (enzyme) function. While antiviral compounds have been disclosed in the present Applicant's earlier filed International Application, published as WO 2016/033660, as modulators of the viral haemagglutinin-neuraminidase the present application provides new compound templates which were not envisaged in that earlier publication which have led to a more complete exploitation of the hPIV HN binding pocket. Certain compounds disclosed herein also extend the inhibitor scaffold to outside of the hPIV HN binding pocket to access gains in beneficial binding interactions for improvements in potency, and for multivalency.

### Definitions

In this patent specification, the terms ‘comprises’, ‘comprising’, ‘includes’, ‘including’, or similar terms are intended to mean a non-exclusive inclusion, such that a method or composition that comprises a list of elements does not include those elements solely, but may well include other elements not listed.

Unless defined otherwise, all technical and scientific terms used herein have the same meaning as would be commonly understood by those of ordinary skill in the art to which this invention belongs.

As used herein, “effective amount” refers to the administration of an amount of the relevant active agent sufficient to prevent the occurrence of symptoms of the condition being treated, or to bring about a halt in the worsening of symptoms or to treat and alleviate or at least reduce the severity of the symptoms. The effective amount will vary in a manner which would be understood by a person of skill in the art with patient age, sex, weight etc. An appropriate dosage or dosage regime can be ascertained through routine trial.

The term “pharmaceutically acceptable salt”, as used herein, refers to salts which are toxicologically safe for systemic or localised administration such as salts prepared from pharmaceutically acceptable non-toxic bases or acids including inorganic or organic bases and inorganic or organic acids. The pharmaceutically acceptable salts may be selected from the group including alkali and alkali earth, ammonium, aluminium, iron, amine, glucosamine, chloride, sulphate, sulphonate, bisulphate, nitrate, citrate, tartrate, bitarate, phosphate, carbonate, bicarbonate, malate, maleate, napsylate, fumarate, succinate, acetate, benzoate, terephthalate, palmoate, piperazine, pectinate and S-methyl methionine salts and the like.

The terms “substituted” and “optionally substituted” in each incidence of its use herein, and in the absence of an explicit listing for any particular moiety, refers to substitution of the relevant moiety, for example an alkyl chain or ring structure, with one or more groups selected from C₁-C₆ alkyl, C₁-C₆ haloalkyl, C₁-C₆ alkoxy, C₁-C₆ haloalkoxy (such as trifluoromethoxy, trifluoroethoxy and the like) CN, OH, oxo, NH₂, NR₂₈R₂₈′ (wherein R₂₈ and R₂₈′ are independently selected from hydrogen, optionally substituted C₁-C₉ alkyl, optionally substituted aryl, R₂₉C═O, R₂₉SO₂, and R₂₉NHC═O wherein R₂₉ is C₁-C₉ alkyl), Cl, F, Br, I, aryl and heterocyclyl which latter two moieties may themselves be optionally substituted. When the term is used before the recitation of a number of functional groups then it is intended to apply to all of the listed functionalities unless otherwise apparent. For example, “optionally substituted amino, heterocyclic, aryl” means all of the amino, heterocyclic and aryl groups may be optionally substituted. In embodiments wherein the relevant group is R₃ and it is linking to another compound of formula (I) to form a dimer, then the recited moiety e.g. “optionally substituted alkyl” or “optionally substituted alkylheteroaryl/alkylheterocyclyl” may be substituted with a linker comprising an alkyl chain and/or a triazole ring through which it is connected to R₃ of the other compound of formula (I) forming the dimer.

The term “alkyl” refers to a straight-chain or branched alkyl substituent containing from, for example, 1 to about 12 carbon atoms, preferably 1 to about 8 carbon atoms, more preferably 1 to about 6 carbon atoms, even more preferably from 1 to about 4 carbon atoms, still yet more preferably from 1 to 2 carbon atoms. Examples of such substituents include methyl, ethyl, propyl, isopropyl, n-butyl, sec-butyl, isobutyl, tert-butyl, pentyl, isoamyl, 2-methylbutyl, 3-methylbutyl, hexyl, heptyl, 2-methylpentyl, 3-methylpentyl, 4-methylpentyl, 2-ethylbutyl, 3-ethylbutyl, octyl, nonyl, decyl, undecyl, dodecyl and the like. The number of carbons referred to relates to the carbon backbone and carbon branching but does not include carbon atoms belonging to any substituents, for example the carbon atoms of an alkoxy substituent branching off the main carbon chain.

The term “cycloalkyl” refers to optionally substituted non-aromatic mono-cyclic, bicyclic or tricyclic carbon groups. Where appropriate, the cycloalkyl group may have a specified number of carbon atoms, for example, C₃-C₆ cycloalkyl is a carbocyclic group having 3, 4, 5 or 6 carbon atoms. Non-limiting examples may include cyclopropyl, cyclobutyl, cyclopentyl, cyclopentenyl, cyclohexyl, cyclohexenyl, cyclohexadienyl and the like. In some embodiments, “cycloalkyl” refers to optionally substituted saturated mono-cyclic, bicyclic or tricyclic carbon groups.

The term “aryl” refers to an unsubstituted or substituted aromatic carbocyclic substituent, as commonly understood in the art. It is understood that the term aryl applies to cyclic substituents that are planar and comprise 4n+2π electrons, according to Hückel's Rule. C-6 aryl is preferred.

The terms “heterocyclic” and “heterocyclyl” as used herein specifically in relation to certain ‘R’ groups refer to a moiety obtained by removing a hydrogen atom from a ring atom of a heterocyclic compound which may have 5 to 7 atoms in the ring and of those atoms between 1 to 4 are heteroatoms, said ring being isolated or fused to a second ring wherein said heteroatoms are independently selected from O, N and S. Heterocyclic and heterocyclyl includes aromatic heterocyclyls and non-aromatic heterocyclyls. Heterocyclic systems may be attached to another moiety via any number of carbon atoms or heteroatoms of the radical and may be both saturated and unsaturated. Heterocyclic systems may be attached to another moiety via any number of carbon atoms or heteroatoms of the radical and may be both saturated and unsaturated. Non-limiting examples of heterocyclic may be selected from pyrazole, imidazole, indole, isoindole, triazole, benzotriazole, tetrazole, pyrimidine, pyridine, pyrazine, diazine, triazine, tetrazine, pyrrolidinyl, pyrrolinyl, pyranyl, piperidinyl, piperazinyl, morpholinyl, tetrahydrofuranyl, tetrahydrothiophenyl, pyrazolinyl, dithiolyl, oxathiolyl, dioxanyl, dioxinyl, oxazinyl, azepinyl, diazepinyl, thiazepinyl, oxepinyl and thiapinyl, imidazolinyl, thiomorpholinyl, and the like.

The terms “heteroaryl” or “aromatic heterocyclyl” refers to an aryl group containing from one or more (particularly one to four) non-carbon atom(s) (particularly N, O or S) or a combination thereof, which heteroaryl group is optionally substituted at one or more carbon or nitrogen atom(s). Heteroaryl rings may also be fused with one or more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5 membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having four heteroatoms (e.g., tetrazoles); 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heretoaryls with three heteroatoms (e.g., 1,3,5-triazine); and 6-membered heteroaryls with four heteroatoms. “Substituted heteroaryl” means a heteroaryl having one or more non-interfering groups as substituents and including those defined under ‘optionally substituted’. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. The group may be a terminal group or a bridging group.

The terms “alkylamine” and “dialkylamine” refer to —NHR and —NRR′ groups, respectively, wherein “R” and “R′” are alkyl, optionally substituted, and may be independently as defined above. That is, R and R′ may be, but are not necessarily, the same alkyl moiety.

The term “amine” may refer to —NH₂, “alkylamine” and “dialkylamine” as defined above.

The term “protected OH” or “protected hydroxy” refers to a hydroxyl group which is protected with a common protecting group such as an acyl group, ether group or ester group including C₁-C₃ acyl, C₁-C₄ alkyl groups to form the ether or aryl, such as benzyl, forming the ether or C₁-C₄ ester.

The term “N-linked” as used herein with reference to compounds of the first aspect including compounds of formula (I) and (II), for example “N-linked triazole”, “N-linked naphthotriazole”, “N-linked indazole” or “N-linked heterocycle”, refers to the moiety attached at the C-4 position of the neuraminic acid core (R₃ in formula (I) and (II)) and limits that attachment to involving a direct attachment between ring carbon and nitrogen atom. Preferably, it refers to the R₃ moiety being linked to the neuraminic acid core via a nitrogen atom which itself forms part of the appropriate heterocycle, such as one of the nitrogens of a triazole ring, indazole, naphthotriazole etc.

Whenever a range of the number of atoms in a structure is indicated (e.g., a C₁-C₁₂, C₁-C₁₀, C₁-C₉, C₁-C₆, C₁-C₄, alkyl, etc.), it is specifically contemplated that any sub-range or individual number of carbon atoms falling within the indicated range also can be used. Thus, for instance, the recitation of a range of 1-12 carbon atoms (e.g., C₁-C₁₂), 1-9 carbon atoms (e.g., C₁—C), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment for a disease or condition caused by viral infection. However, it will be understood that the aforementioned terms do not imply that symptoms are necessarily present.

References herein to “haemagglutinin-neuraminidase”, “haemagglutinin-neuraminidase protein” and the like may be considered interchangeable with “haemagglutinin and/or neuraminidase functions”. They may be considered to incorporate one or both of blocking of the haemagglutination function or inhibition of the neuraminidase (enzyme) function. The blocking of the haemagglutination function may therefore involve modulation, blocking or inhibition of the haemagglutinin-neuraminidase protein which may, without wishing to be bound by any theory, be one mechanism of action of the compounds described herein.

According to a first aspect of the invention, there is provided a compound of formula (I), or a pharmaceutically acceptable salt thereof:

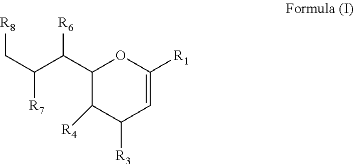

wherein, R₁ is selected from the group consisting of COOH, or a salt thereof, C(O)NR₉R₁₀, C(O)OR₁₁ wherein R₉, R₁₀ and R₁₁ are independently selected from the group consisting of hydrogen, optionally substituted C₁-C₆ alkyl and optionally substituted aryl;

R₃ is selected from the group consisting of optionally substituted N-linked naphthotriazole, optionally substituted N-linked indazole, and N-linked triazole of the following formula:

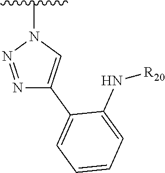

wherein R₂₀ is selected from the group consisting of

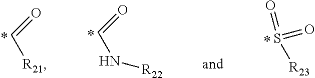

wherein, * is the point of attachment, R₂₁, R₂₂ and R₂₃ are independently selected from the group consisting of optionally substituted alkyl, optionally substituted alkenyl, substituted alkynyl, optionally substituted cycloalkyl, optionally substituted aryl, optionally substituted heteroaryl, optionally substituted heterocyclyl, optionally substituted alkylheterocyclic, optionally substituted alkylheteroaryl, optionally substituted alkylamine, optionally substituted dialkylamine and an optionally substituted linker which links the compound to another compound of Formula (I);

R₄ is selected from the group consisting of sulfonamide, urea and NHC(O)R₁₇ wherein R₁₇ is selected from the group consisting of C₁-C₆ alkyl, C₁-C₆ haloalkyl and C₃-C₆ cycloalkyl, all of which may be optionally substituted;

R₆, R₇ and R₈ are independently selected from the group consisting of OH, protected OH, NH₂, C₁-C₆ alkyl, C₁-C₆ haloalkyl, NR₁₈R₁₈′, C₁-C₆ alkoxy, C₁-C₆ haloalkoxy, —OC(O)R₁₈, —NH(C═O)R₁₈, and S(O)ₙR₁₈, wherein n=0-2 and each R₁₈ and R₁₈′ are independently selected from hydrogen, optionally substituted C₁-C₆ alkyl and optionally substituted C₁-C₉ alkanoyl, as appropriate.

In one embodiment of the first aspect, the compound of formula (I) is a compound of formula (II):

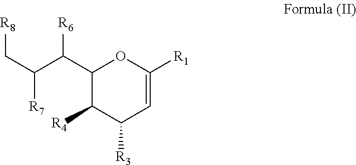

wherein, R₁, R₃, R₄, R₆, R₇ and R₈ are as previously described.

In one embodiment of the compound of formula (I) or (II) R₁ is COOH, or a salt thereof, or C(O)OR₁₁ wherein R₁₁ is selected from methyl, ethyl and propyl.

In certain specific embodiments R₁ is selected from the group consisting of COOH, COONa and C(O)OMe.

In one embodiment of the compound of formula (I) or (II), when R₃ is optionally substituted N-linked naphthotriazole it is of the following formula:

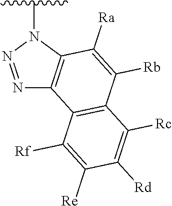

wherein, Rₐ, R_{b}, R_{c}, R_{d}, Rₑ, and R_{f} are independently selected from the group consisting of hydrogen, hydroxyl, cyano, halo, amido, C₁-C₁₂ alkyl, C₁-C₁₂ alkoxy, C₁-C₁₂ haloalkoxy, C₁-C₁₂ alkanoyl, C₁-C₁₂ haloalkanoyl, C₁-C₁₂ haloalkyl, pyridyl and phenyl, all of which groups may be optionally substituted as appropriate.

In certain embodiments, Rₐ, R_{b}, R_{c}, R_{d}, Rₑ, and R_{f} are independently selected from the group consisting of hydrogen, hydroxyl, cyano, halo, acetamido, C₁-C₆ alkyl, C₁-C₉ alkoxy, C₁-C₉ alkanoyl, C₁-C₆ haloalkyl, optionally substituted pyridyl and optionally substituted phenyl.

In certain embodiments, one or more of Rₐ and R_{b}; R_{b} and R_{c}; R_{c} and R_{d}; R_{d} and Rₑ; and Rₑ, and R_{f} may form a 5- or 6-membered aryl or heteroaryl or heterocyclic ring.

In one embodiment of the compound of formula (I) or (II), when R₃ is optionally substituted N-linked indazole it is of the following formula:

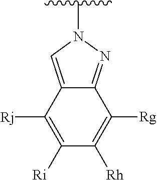

wherein, R_{g}, Rₕ, Rᵢ, and Rⱼ are independently selected from the group consisting of hydrogen, hydroxyl, halo, C₁-C₆ alkoxy, C₁-C₆ haloalkyl, cyano, sulfonyl, amine, amido, and carboxyl; and

R_{g} and Rₕ, Rₕ and Rᵢ, and Rᵢ and Rⱼ may together form a heteroaryl, heterocyclic or aryl ring, each of which may be optionally substituted.

In embodiments, R_{g} and Rₕ, Rₕ and Rᵢ, and Rᵢ and Rⱼ may together form a 5-, 6- or 7-membered: heteroaryl, heterocyclic or aryl ring (especially a 5 or 6 membered heteroaryl, heterocyclic or aryl ring; more especially 1,3-dioxolane, pyridine, thiophene, imidazole, pyrrole or phenyl), each of which may be optionally substituted (especially by at least one of halo and cyano; more especially by at least one of F, Br and cyano).

In certain embodiments, R_{g} is selected from the group consisting of hydrogen, hydroxyl, cyano, halo (including fluoro), C₁-C₆ alkoxy, amido, and carboxyl.

In embodiments, Rₕ is selected from the group consisting of hydrogen, hydroxyl, halo (including fluoro or bromo), cyano, C₁-C₆ haloalkyl, C₁-C₆ alkoxy, sulfonyl, carboxyl, and amine.

In embodiments, when any one or more of R_{g}, Rₕ, Rᵢ, and Rⱼ are amine then they may be alkylamine or dialkylamine.

In certain embodiments, R_{g} and Rₕ may together form a 6 membered heteroaryl or aryl ring, each of which may be optionally substituted.

In embodiments, Rᵢ is selected from the group consisting of hydrogen, hydroxyl, halo, C₁-C₆ haloalkyl, C₁-C₆ alkoxy, cyano, and carboxyl.

In certain embodiments, Rₕ and Rᵢ may together form a 5- or 6-membered heterocyclic, heteroaryl or aryl ring (for example a 1,3-dioxolane), each of which may be optionally substituted.

In embodiments, Rⱼ is selected from the group consisting of hydrogen, hydroxyl, halo, C₁-C₆ alkoxy, amido, and carboxy.

In certain embodiments, Rᵢ and Rⱼ may together form a 5 or 6 membered heteroaryl or aryl ring, each of which may be optionally substituted. In some embodiments, Rᵢ and Rⱼ may together form a pyrrole, pyridine, thiophene or imidazole ring (especially a pyridine, thiophene or imidazole ring) which may be optionally substituted (especially by at least one of halo and cyano; more especially by at least one of F, Br and cyano).

In embodiments, R_{g} is selected from the group consisting of hydrogen, hydroxyl, Br, F, methoxy, ethoxy, acetamido, and carboxyl.

In embodiments, Rₕ is selected from the group consisting of hydrogen, hydroxyl, Br, F, trifluoroalkyl, methoxy, ethoxy, methylsulfonyl, cyano, carboxyl, dimethylamine and diethylamine.

In certain embodiments, R_{g} and Rₕ may together form an optionally substituted phenyl ring.

In embodiments, Rᵢ is selected from the group consisting of hydrogen, hydroxyl, Br, F, methoxy, ethoxy, cyano, and carboxyl.

In certain embodiments, Rₕ and Rᵢ may together form a 5-membered oxygen-containing heterocycle or a phenyl ring, each of which may be optionally substituted.

In embodiments, Rⱼ is selected from the group consisting of hydrogen, hydroxyl, Br, F, methoxy, ethoxy, acetamido, and carboxy.

In certain embodiments, Rᵢ and Rⱼ may together form a phenyl ring, each of which may be optionally substituted.

In embodiments wherein R₃ is N-linked triazole, as defined above, of the following formula:

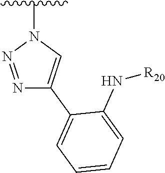

wherein R₂₀ is selected from the group consisting of

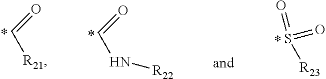

then, in embodiments, R₂₁ may be selected from the group consisting of optionally substituted C₁-C₁₂ alkyl, optionally substituted C₁-C₁₂ alkenyl, optionally substituted 5 or 6 membered aryl, optionally substituted C₁-C₁₂ alkyl-nitrogenheterocycle, optionally substituted C₁-C₉ alkyl-nitrogenheteroaryl, optionally substituted C₁-C₁₂ alkylamine, optionally substituted C₁-C₁₂ dialkylamine, optionally substituted C₁-C₆ alkyl-NH—CO-aryl, optionally substituted C₁-C₆ alkyl-NH—CO-aryl-aryl, optionally substituted C₁-C₆ alkyl-NH—CO-cycloalkyl, optionally substituted C₁-C₆ alkyl-NH—SO₂-aryl, optionally substituted C₁-C₆ alkyl-NH—SO₂-C₁-C₆alkyl-aryl, and an optionally substituted linker which links the compound to another compound of Formula (I).

In embodiments, R₂₁, R₂₂ and R₂₃ are independently selected from the group consisting of optionally substituted C₁-C₁₂ alkyl, optionally substituted C₂-C₁₂ alkenyl, optionally substituted C₂-C₁₂ alkynyl, optionally substituted C₆ cycloalkyl, optionally substituted 5 or 6-membered aryl, optionally substituted 5 or 6-membered heteroaryl, optionally substituted 5 or 6-membered heterocyclyl, optionally substituted C₁-C₉ alkyl 5 or 6-membered heterocyclic, optionally substituted C₁-C₉ alkyl 5 or 6-membered heteroaryl, optionally substituted C₁-C₁₂ alkylamine, optionally substituted C₁-C₁₂ dialkylamine, optionally substituted C₁-C₆ alkyl-NH—CO-aryl, optionally substituted C₁-C₆ alkyl-NH—CO-aryl-aryl, optionally substituted C₁-C₆ alkyl-NH—CO-cycloalkyl, optionally substituted C₁-C₆ alkyl-NH—SO₂-aryl, optionally substituted C₁-C₆ alkyl-NH—SO₂—C₁-C₆alkyl-aryl, and an optionally substituted linker which links the compound to another compound of Formula (I).

In embodiments, R₂₁, R₂₂ and R₂₃ are independently selected from the group consisting of optionally substituted C₁-C₉ alkyl, optionally substituted C₂-C₉ alkenyl, optionally substituted C₂-C₉ alkynyl, optionally substituted C₆ cycloalkyl, optionally substituted 5 or 6-membered aryl, optionally substituted 5 or 6-membered nitrogen heteroaryl, optionally substituted 5 or 6-membered nitrogen heterocyclyl, optionally substituted C₁-C₆ alkyl 5 or 6-membered heterocyclic, optionally substituted C₁-C₆ alkyl 5 or 6-membered nitrogen heteroaryl, optionally substituted C₁-C₉ alkylamine, optionally substituted C₁-C₉ dialkylamine, optionally substituted C₁-C₆ alkyl-NH—CO-phenyl, optionally substituted C₁-C₆ alkyl-NH—CO-phenyl-phenyl, optionally substituted C₁-C₆ alkyl-NH—CO-(3 to 6 membered)cycloalkyl, optionally substituted C₁-C₆ alkyl-NH—SO₂-phenyl, optionally substituted C₁-C₆ alkyl-NH—SO₂—C₁-C₆alkyl-phenyl, and an optionally substituted linker which links the compound to another compound of Formula (I).

In particular embodiments, R₂₁ may be selected from optionally substituted C₁-C₉ alkyl, C₂-C₉ alkenyl, and C₂-C₉ alkynyl with the terminal carbon of the relevant chain connecting to a moiety selected from the group consisting of azido, optionally substituted amino, and optionally substituted 5-membered nitrogen heteroaryl. Preferably, the optionally substituted 5-membered nitrogen heteroaryl is selected from the group consisting of pyrrole, imidazole, pyrazole, triazole, tetrazole, benzotriazole and isoindole, each of which may be optionally substituted as appropriate. More preferably the optionally substituted 5-membered nitrogen heteroaryl is optionally substituted triazole.

In particular embodiments, R₂₁ may be selected from optionally substituted C₁-C₉ alkyl, C₂-C₉ alkenyl, C₂-C₉ alkynyl, optionally substituted C₁-C₆alkyl amino, optionally substituted phenyl (in which the optional substituents may especially be selected from at least one of the group consisting of: halo, —OC₁-C₆alkyl, O—C₁-C₆-haloalkyl, nitro, and C₁-C₆-alkyl), optionally substituted C₁-C₆alkyl-NHCO-phenyl (in which the phenyl may especially be optionally substituted with at least one of the group consisting of: —N(C₁-C₆ alkyl)₂ and phenyl), optionally substituted C₁-C₆alkyl-NHSO₂-phenyl (in which the phenyl may especially be optionally substituted with nitro), optionally substituted C₁-C₆alkyl-NHSO₂-C₁-C₆ alkyl-phenyl, and optionally substituted C₁-C₆alkyl-NHCO-(3 to 6 membered)cycloalkyl.

In particular embodiments, R₂₃ may be selected from C₁-C₆ alkyl and optionally substituted phenyl (in which the optional substituents may especially be nitro).

When R₂₁ is an optionally substituted linker which links the compound to another compound of Formula (I) then the linker may be selected from optionally substituted C₁-C₁₂ alkyl; optionally substituted C₁-C₉ alkyl; optionally substituted C₂-C₉ alkenyl; and optionally substituted C₂-C₉ alkynyl; any of which may be linked to a 5-membered nitrogen heteroaryl. Suitably, the 5-membered nitrogen heteroaryl may be triazole.

When R₂₁ is an optionally substituted linker which links the compound to another compound of Formula (I) then the compound of formula (I) may be of the following formula:

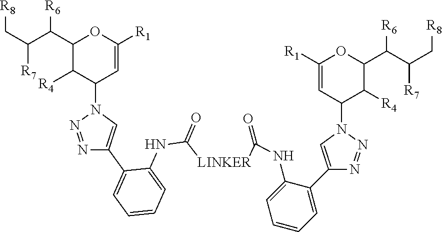

wherein, R₁, R₄, R₆, R₇ and R₈ are as previously described and LINKER is selected from C₁-C₁₂ alkyl; C₁-C₉ alkyl; C₂-C₉ alkenyl; and C₂-C₉ alkynyl; any of which may be optionally substituted and optionally linked to a 5-membered nitrogen heteroaryl.

The optional substituents may be as previously defined with one or more of hydroxyl, aryl, heteroaryl, amido and ether being particularly preferred.

In certain embodiments, LINKER is selected from the following: C₁-C₁₂ alkyl, C₁-C₉ alkyl, C₂-C₉ alkenyl, C₂-C₉ alkynyl,

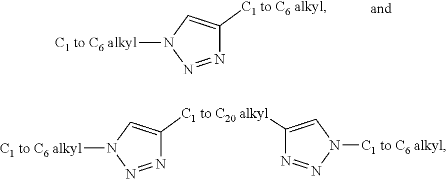

wherein the C₁-C₁₂ alkyl, C₁-C₆ alkyl and C₁-C₂₀ alkyl moieties referred to may all be optionally substituted with one or more of hydroxyl, aryl, heteroaryl, amido and ether.

In the above structure having C₁ to C₂₀ alkyl linking the two triazole rings, the C₁ to C₂₀ alkyl may, in embodiments, be selected from the group consisting of C₁ to C₁₆ alkyl, C₁ to C₁₂ alkyl, C₁ to C₉ alkyl and C₁ to C₆ alkyl, which may all be optionally substituted with one or more of aryl, heteroaryl, amido and ether.

In certain embodiments, R₃ may be selected from the group consisting of:

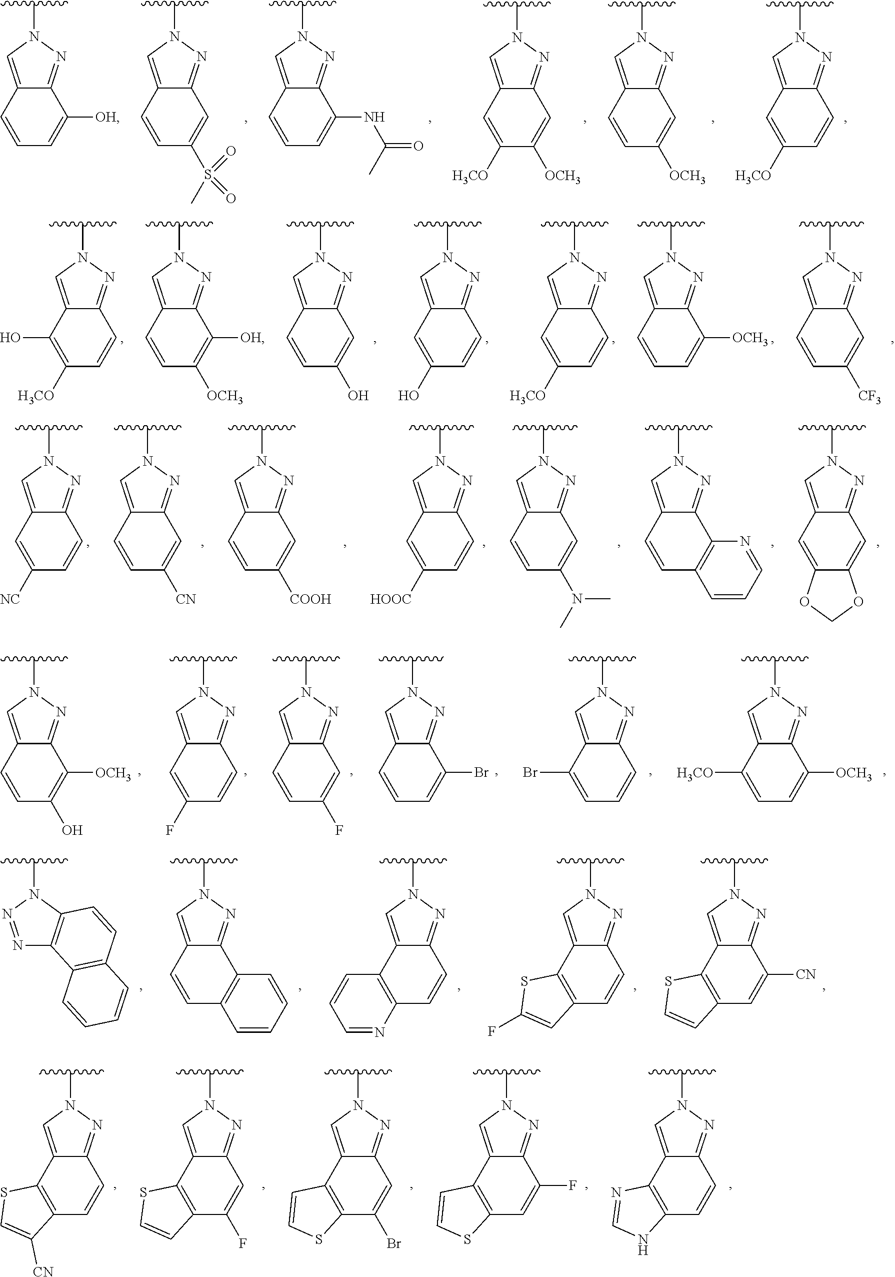

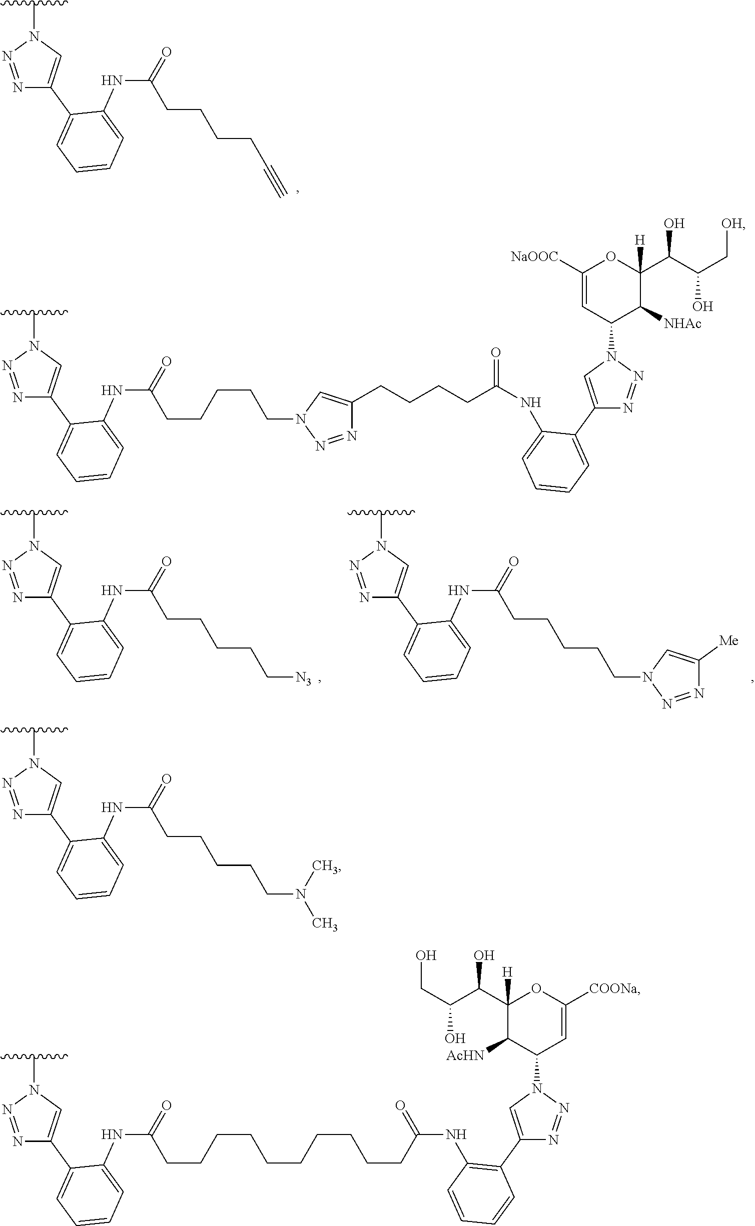

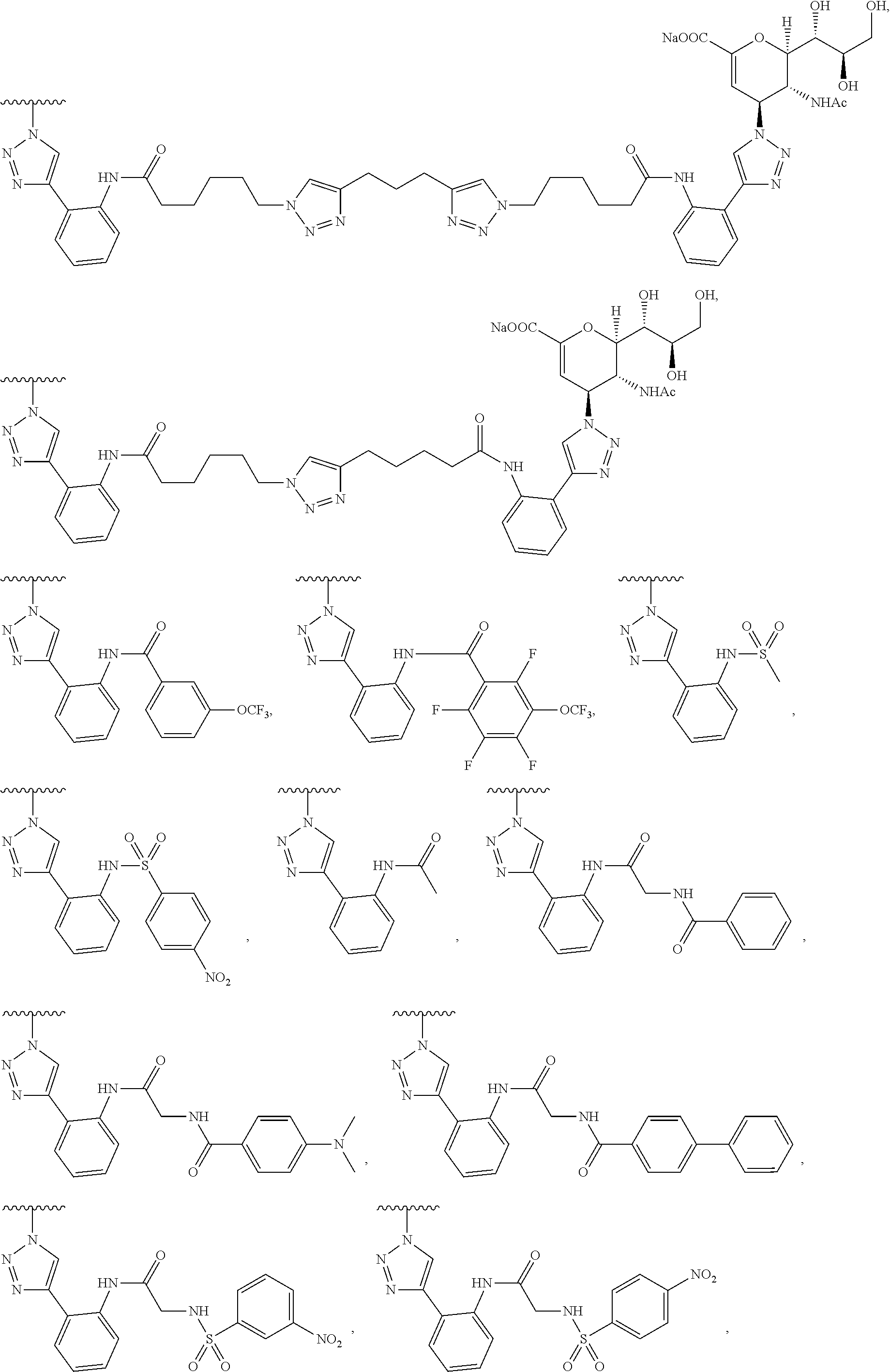

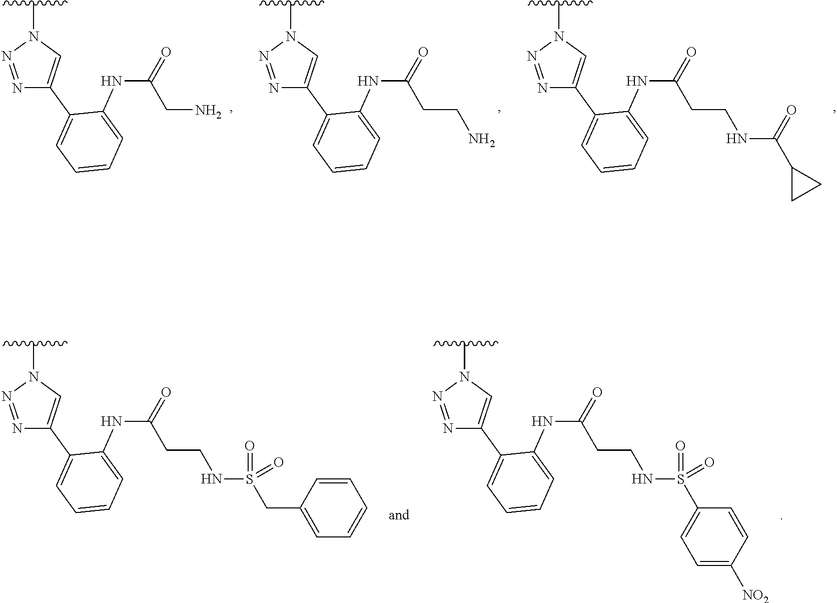 [SMILES: *n1cc(-c2ccccc2NC(=O)CN)nn1 ; *n1cc(-c2ccccc2NC(=O)CCN)nn1 ; *n1cc(-c2ccccc2NC(=O)CCNC(=O)C2CC2)nn1 ; *n1cc(-c2ccccc2NC(=O)CCNS(=O)(=O)Cc2ccccc2)nn1 ; *n1cc(-c2ccccc2NC(=O)CCNS(=O)(=O)c2ccc([N+](=O)[O-])cc2)nn1]

The specific moieties or the disclosure of any R₃ group listed above may be combined with any disclosure of an R₁, R₄, R₆, R₇ or R₈ group as described herein.

In any of the previously described embodiments, R₄ may be selected from the group consisting of —NHS(O)₂R₂₇ wherein R₂₇ is selected from the group consisting of C₁-C₆ alkyl, C₁-C₆ haloalkyl and C₃-C₆ cycloalkyl, all of which may be optionally substituted, —NHC(O)NHR₁₇ wherein R₁₇ may be as previously defined, and the following:

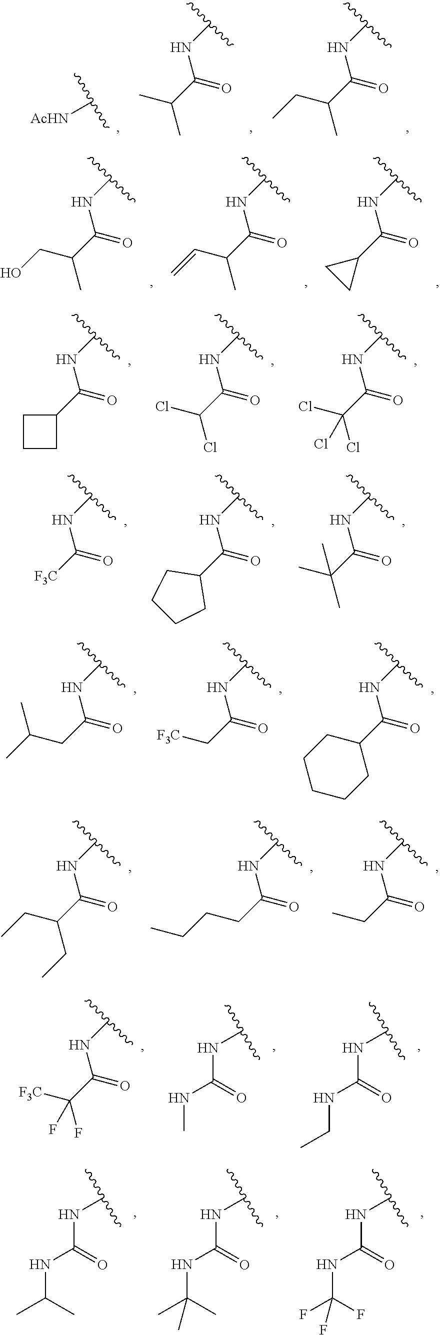

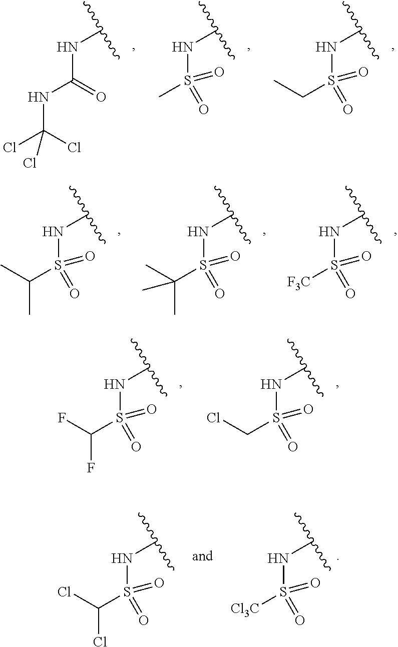

In embodiments of any of the formulae described for the first aspect, R₄ is selected from the group consisting of —NHAc, —NHC(O)CH(CH₃)₂, —NHC(O)CF₃ and —NHC(O)CH₂CH₃.

In any embodiment of the compounds of formula (I) or (II), R₆, R₇ and R₈ may be independently selected from the group consisting of OH, C₁-C₃ alkoxy, —OC(O)R₁₈ wherein R₁₈ is optionally substituted C₁-C₃ alkyl, and —NR₁₈R₁₈′ wherein R₁₈ and R₁₈′ are selected from hydrogen, optionally substituted C₁-C₃ alkyl and optionally substituted C₁-C₆ alkanoyl. When R₁₈ is C(O)R (i.e. alkanoyl) then ‘R’ may be C₁-C₆ alkyl or C₅-C₆ cycloalkyl.

In embodiments of any of the formulae described for the first aspect, R₆, R₇ and R₈ may be independently selected from OH and OAc.

In one embodiment, the compound of formula (I) may be a compound of formula (IIIa) or (IIIb):

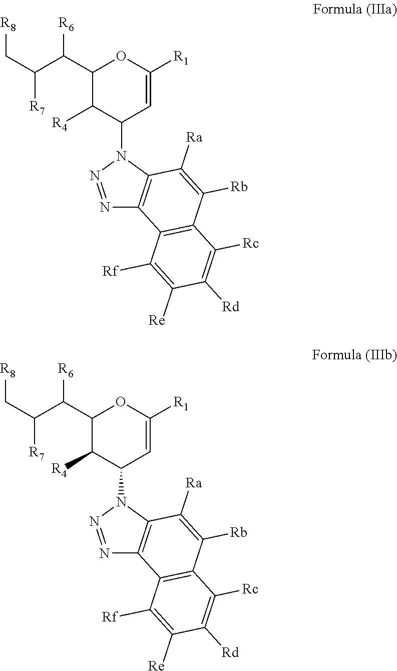

wherein, R₁, R₄, R₆, R₇, R₈, Rₐ, R_{b}, R_{c}, R_{d}, Rₑ, and R_{f} are as previously described.

In certain embodiments of formula (IIIa) and formula (IIIb), R₁ may be selected from COOH, or a salt thereof, or C(O)OR₁₁ wherein R₁₁ is selected from methyl, ethyl and propyl, preferably R₁ is selected from COOH, COONa and C(O)OMe; R₄ is selected from the group consisting of —NHAc, —NHC(O)CH₂(CH₃)₂, —NHC(O)CF₃, —NHC(O)CH₂CH₃, —NHS(O)₂R₂₇ wherein R₂₇ is selected from the group consisting of C₁-C₆ alkyl, C₁-C₆ haloalkyl and C₃-C₆ cycloalkyl, all of which may be optionally substituted, —NHC(O)NHR₁₇ wherein R₁₇ may be as previously defined; and R₆, R₇ and R₈ may be independently selected from OH and OAc.

In one embodiment, the compound of formula (I) may be a compound of formula (IVa) or (IVb):

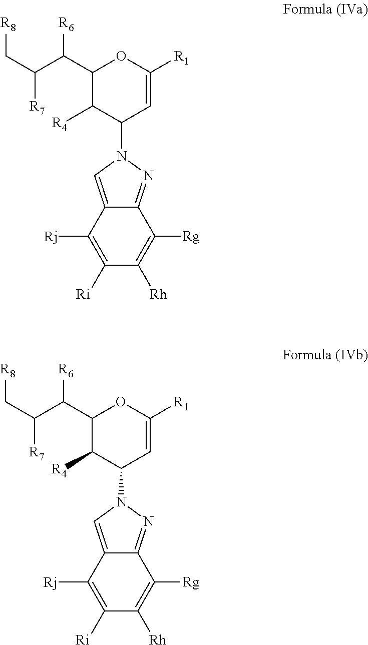

wherein, R₁, R₄, R₆, R₇, R₈, R_{g}, Rₕ, Rᵢ, and Rⱼ are as previously described.

In certain embodiments of formula (IVa) and formula (IVb), R₁ may be selected from COOH, or a salt thereof, or C(O)OR₁₁ wherein R₁₁ is selected from methyl, ethyl and propyl, preferably R₁ is selected from COOH, COONa and C(O)OMe; R₄ is selected from the group consisting of —NHAc, —NHC(O)CH₂(CH₃)2, —NHC(O)CF₃ and —NHC(O)CH₂CH₃; and R₆, R₇ and R₈ may be independently selected from OH and OAc.

In one embodiment, the compound of formula (I) may be a compound of any one or more of formulae Va, Vb, VIa, VIb, VIIa and VIIb:

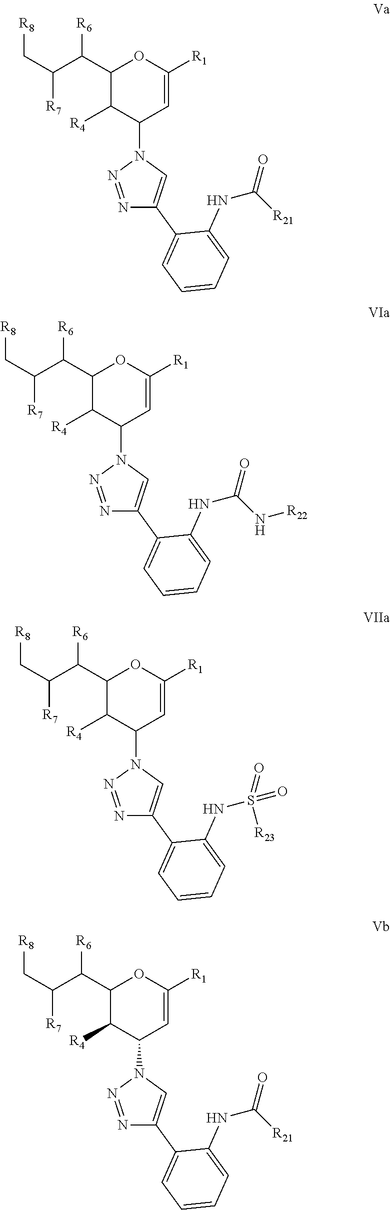

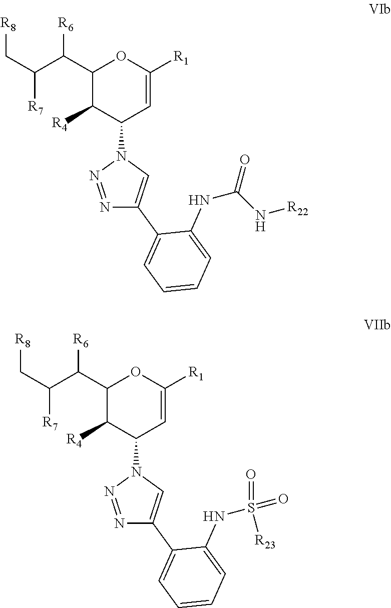

wherein, R₁, R₄, R₆, R₇, R₈, R₂₁, R₂₂ and R₂₃ areas previously defined for anyone or more embodiments of the first aspect.

In one embodiment of formula (Va) and (Vb), the compound is of the below formula:

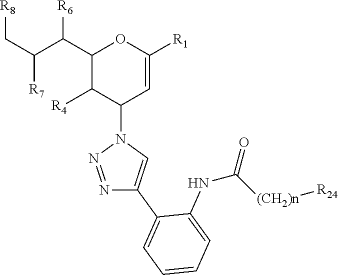

wherein n is between 1 and 9, preferably 1 and 6 and wherein R₂₄ is selected from the group consisting of azido; 5-membered nitrogen heteroaryl optionally fused with a further ring system; COOR₃₀ wherein R₃₀ is selected from hydrogen, C₁-C₁₂ alkyl and aryl; and —NR₂₅R₂₆ wherein R₂₅ and R₂₆ are independently selected from hydrogen and C₁-C₆ alkyl.

In embodiments, when R₂₄ is a 5-membered nitrogen heteroaryl optionally fused with a further ring system then it may be fused with a 5- or 6-membered aryl ring including a phenyl ring. For example, isoindole and similar fused ring systems may be formed.

Compounds of the above formula may be suitable for use in forming the dimers of compounds of formula (I) as described herein. That is, certain compounds of formula Va and Vb, in particular, may be useful for conversion into dimers of formula (I).

In embodiments of formula (I) and formula (II) the compound may be selected from the group consisting of:

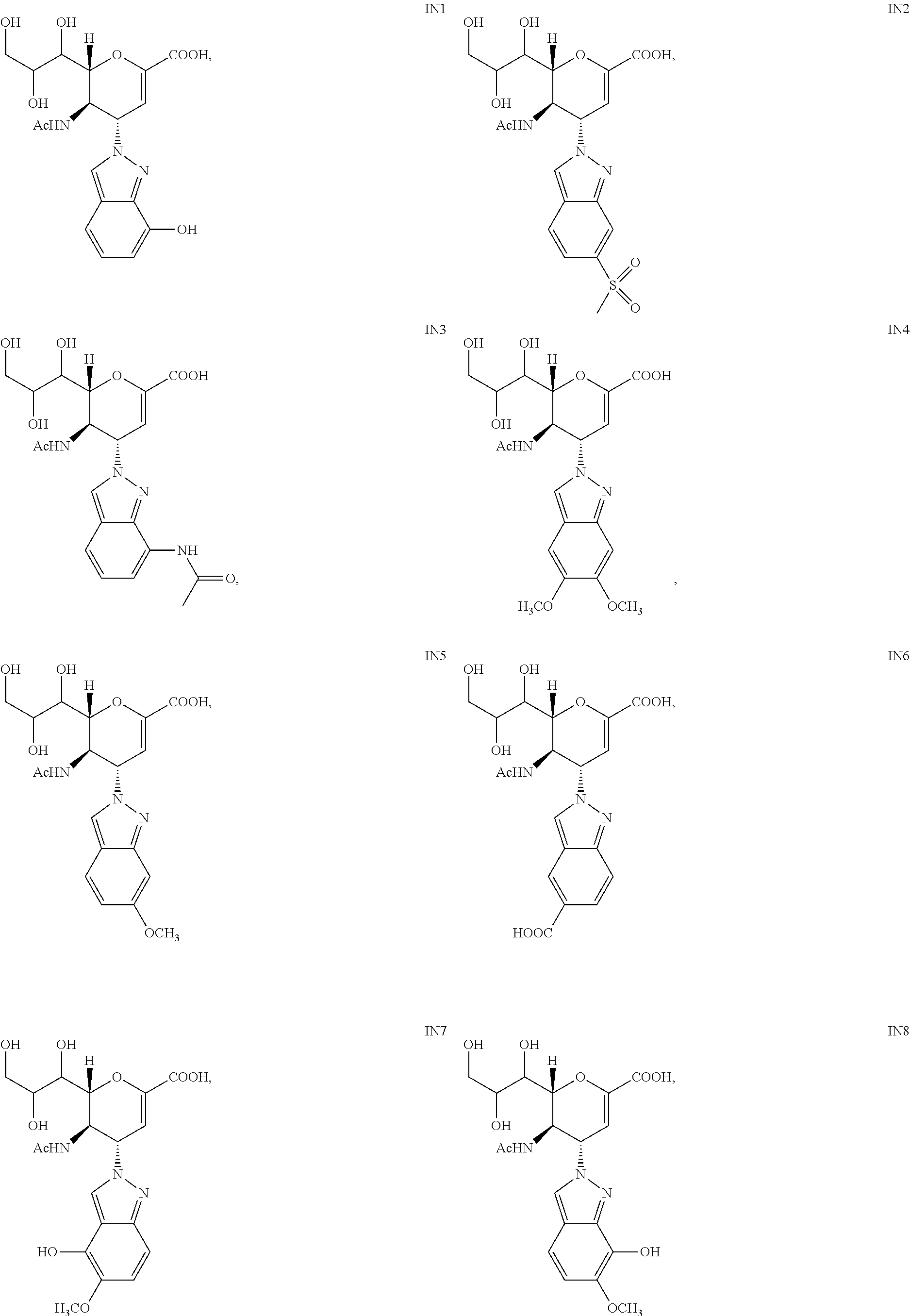

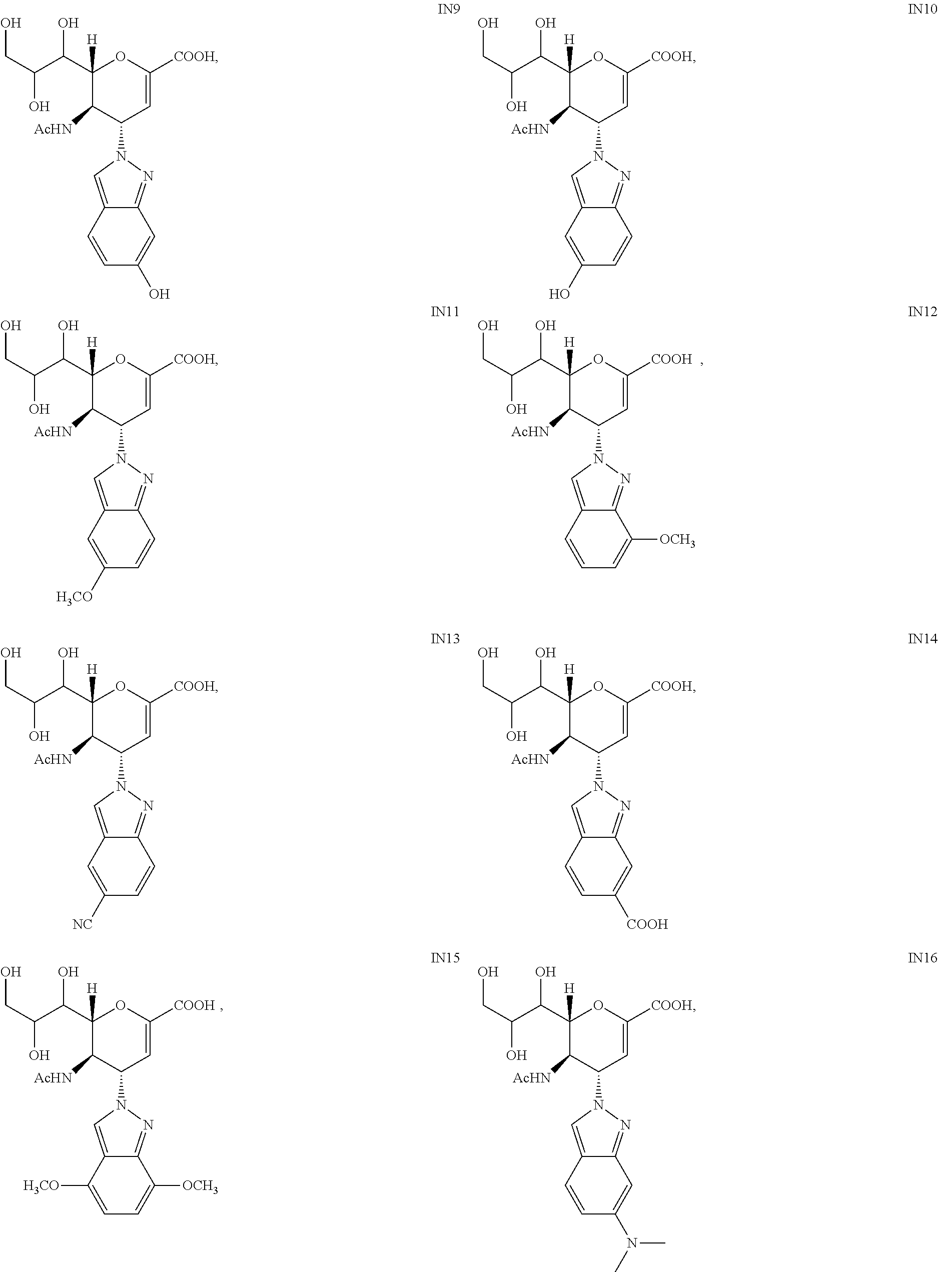

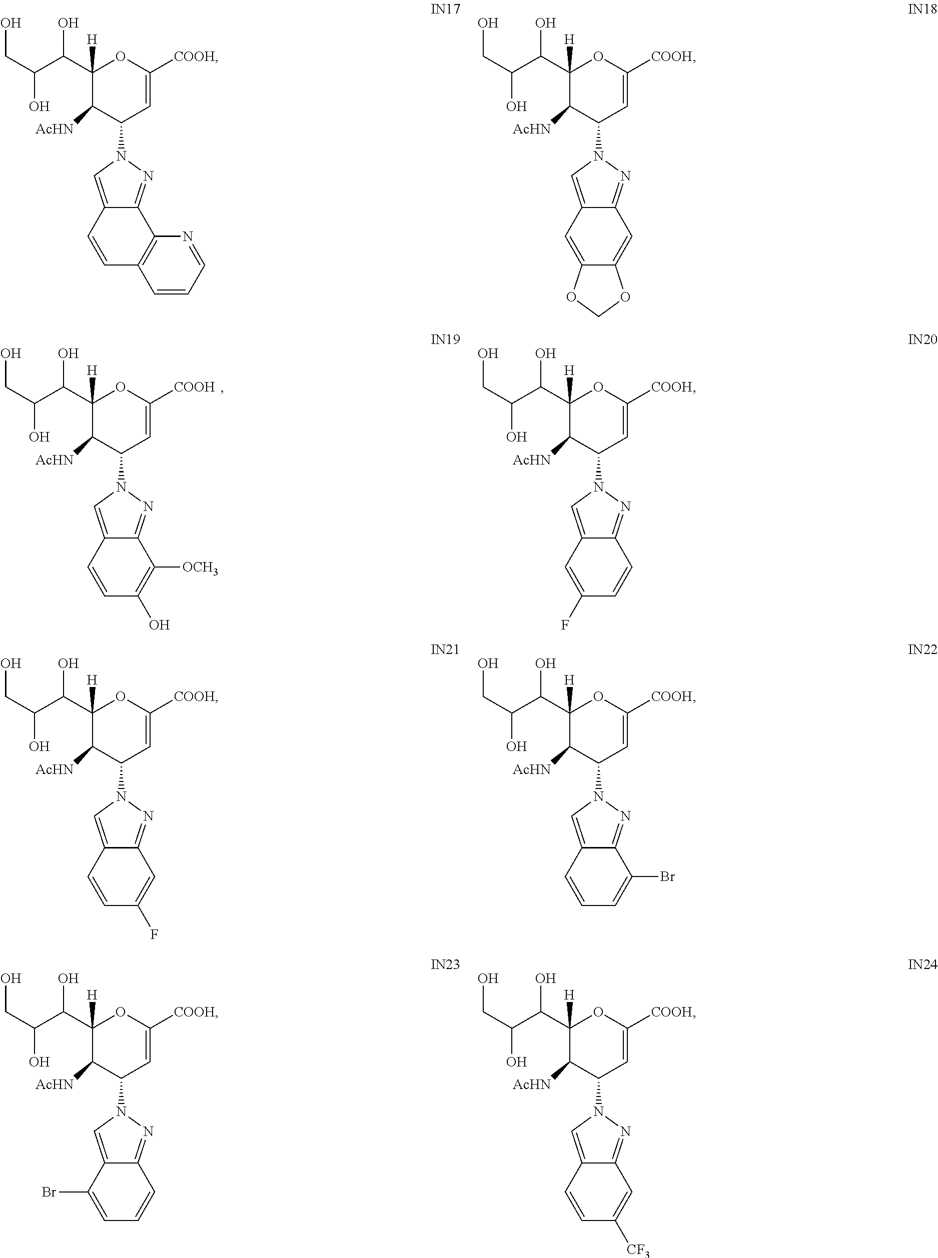

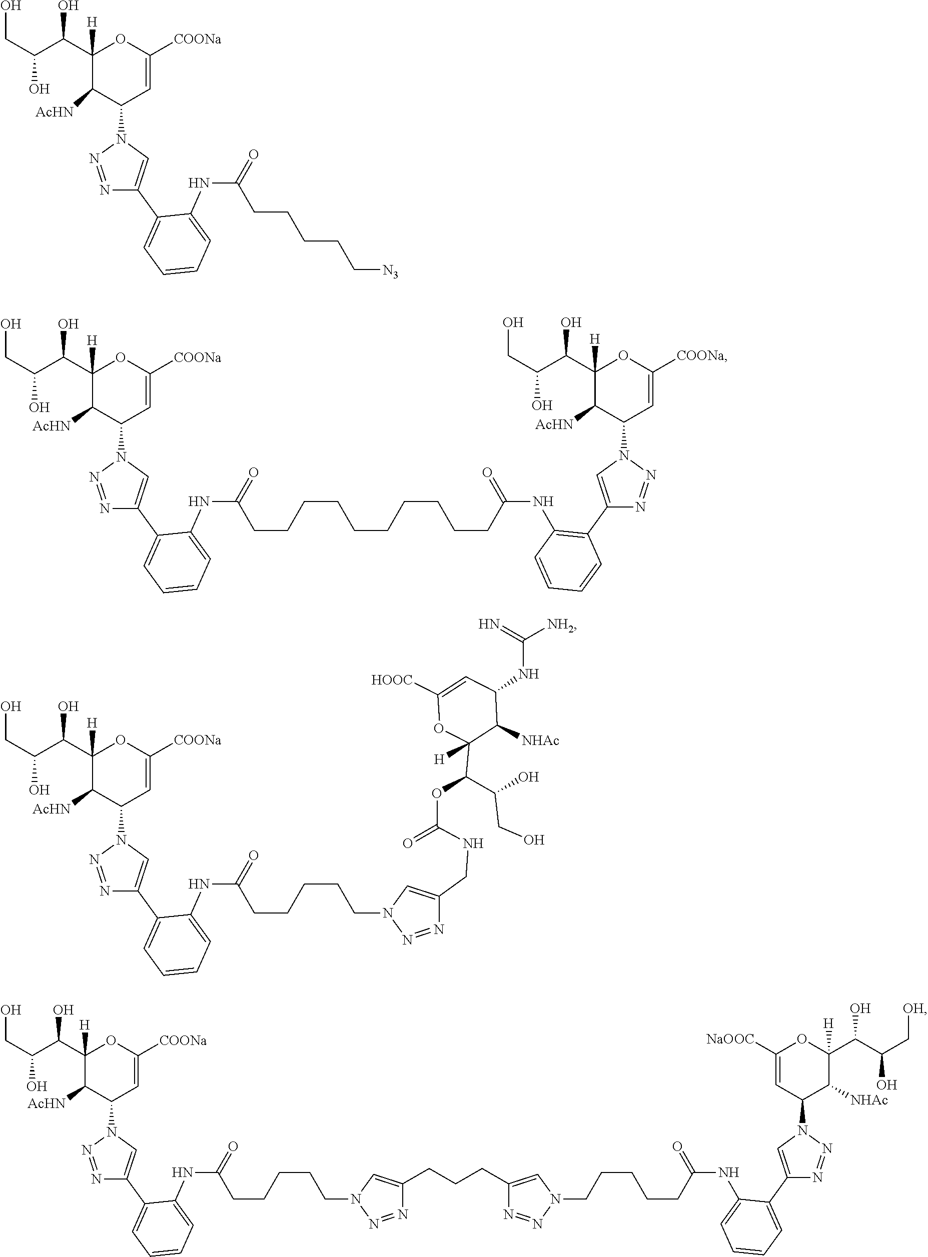

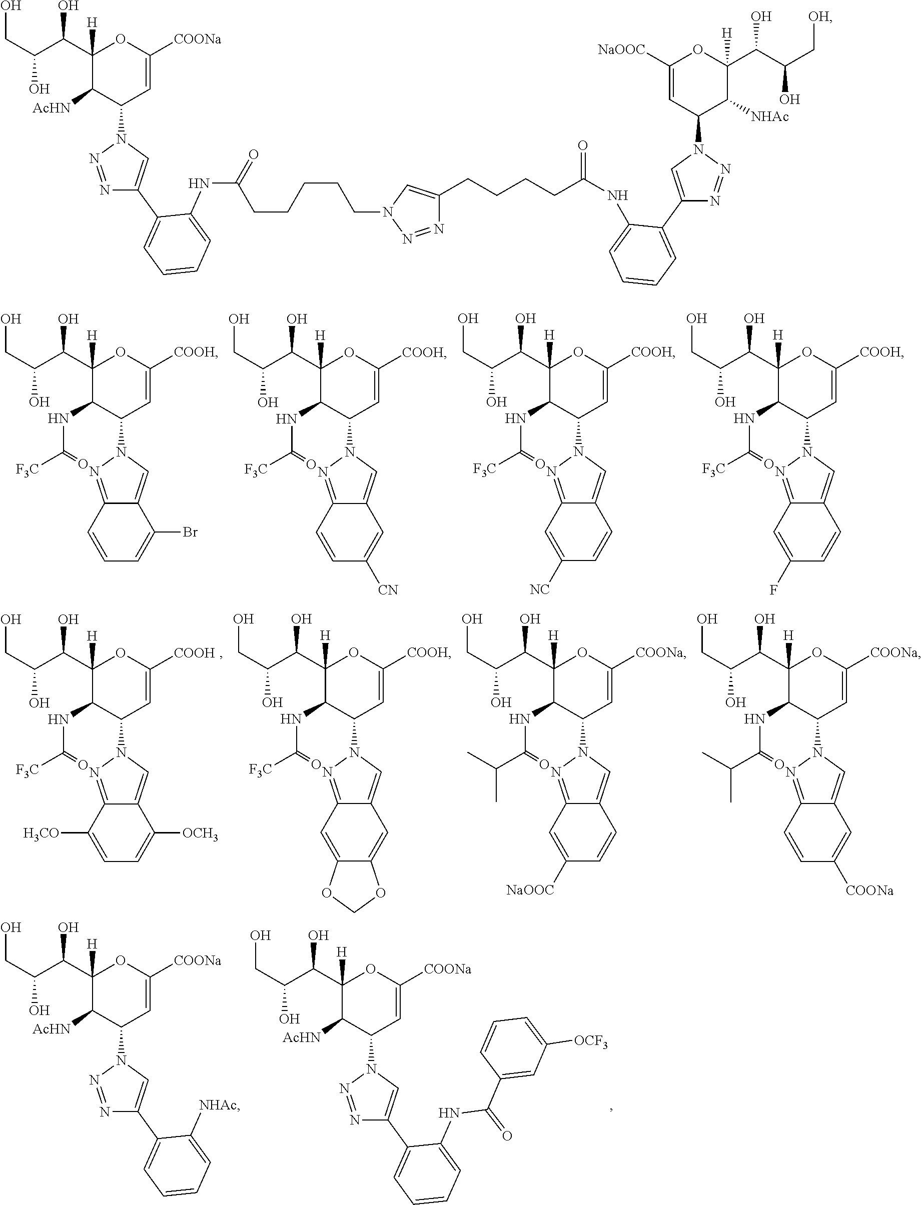

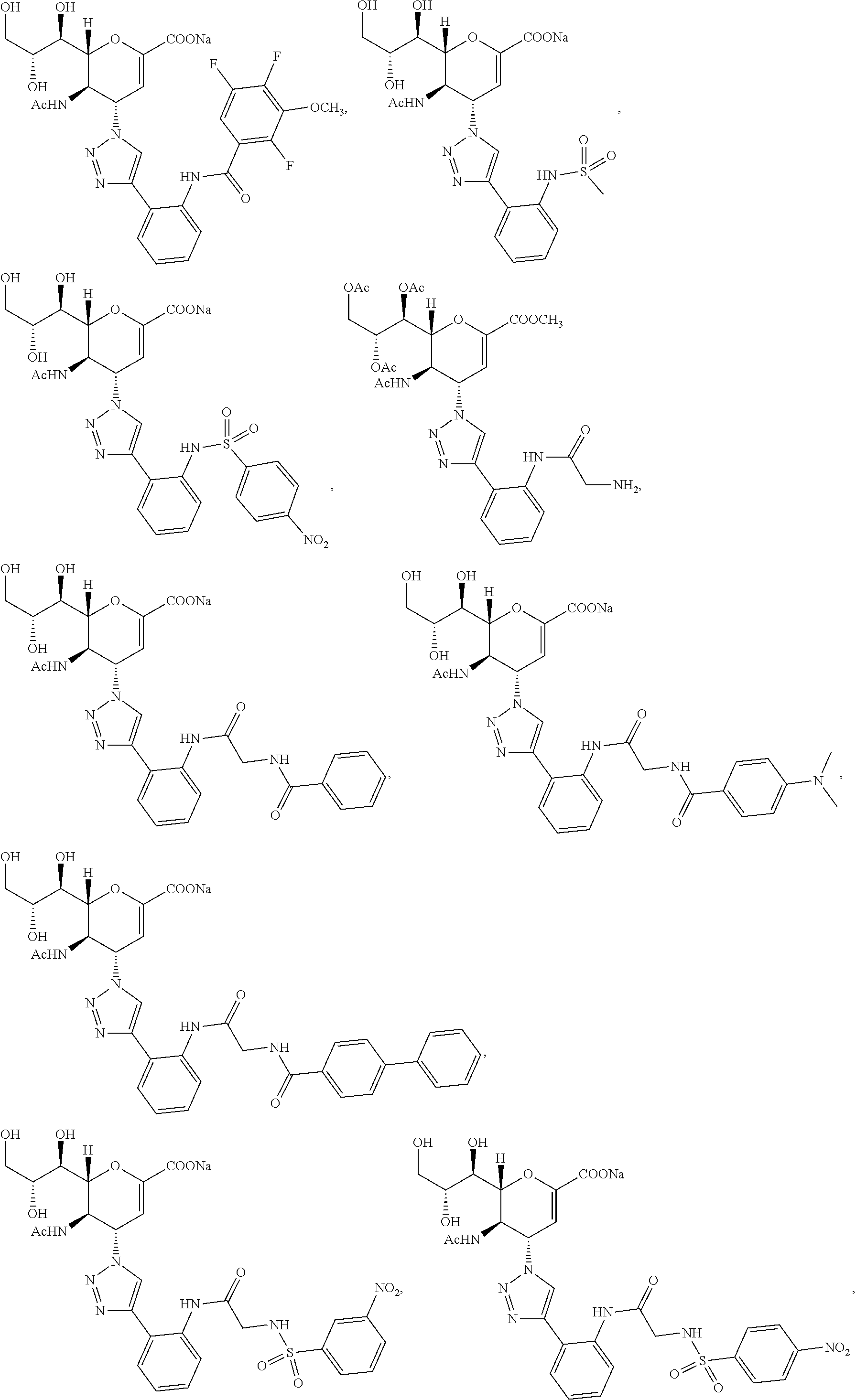

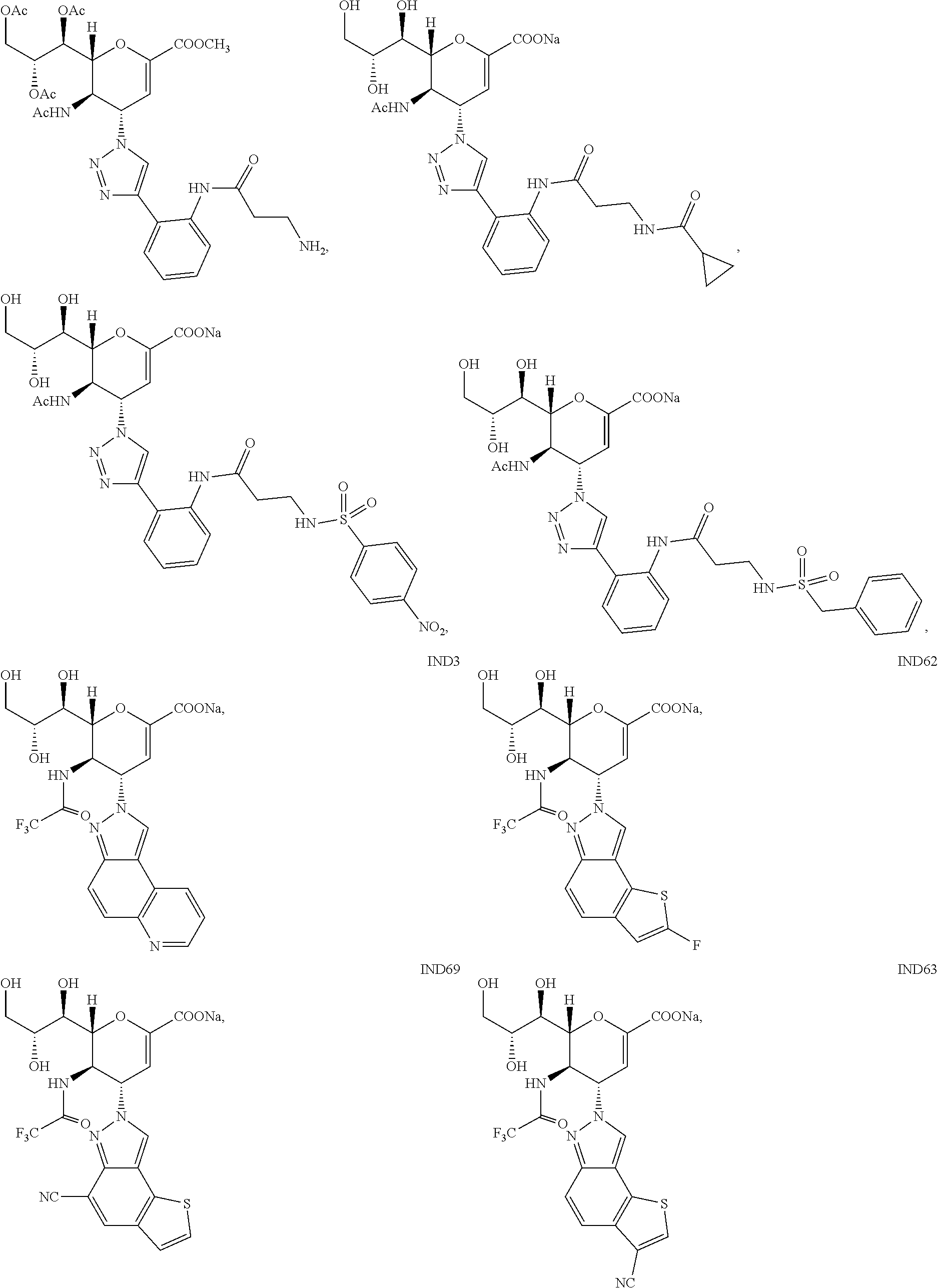

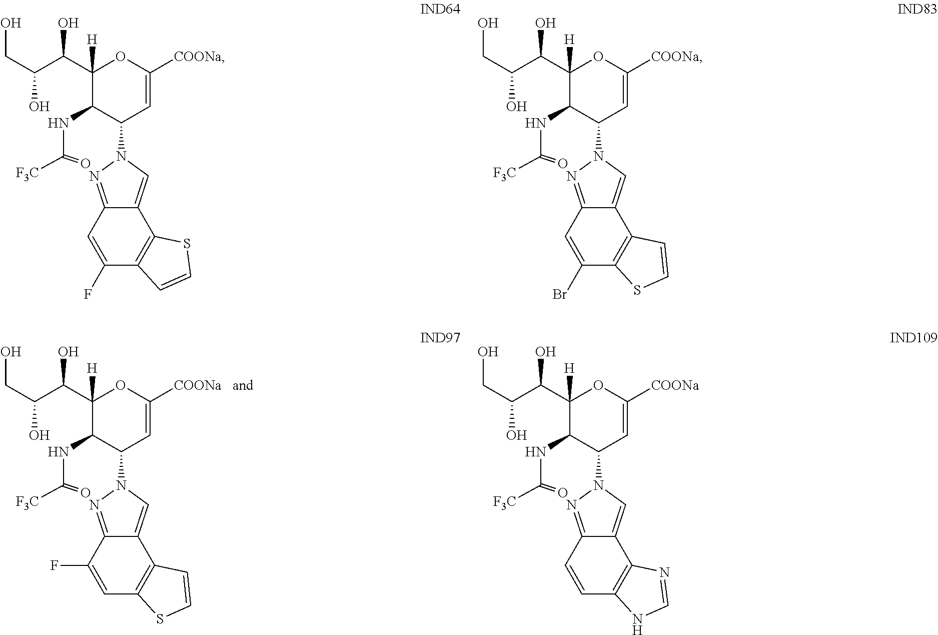

and protected forms thereof, including acetyl replacing hydrogen at the free hydroxyls, all C-2 analogues thereof wherein the C-2 carboxy group is in the protonated form, sodium salt form or prodrug form and wherein each compound may be considered to have close analogues disclosed wherein the R₄ position is explicitly replaced with any —NHC(O)R group wherein R is C₁-C₄ alkyl or haloalkyl.

It will be appreciated by a person of skill in the art of synthetic chemistry that the COOH group is easily interchanged with a salt form or an ester protecting group, for example a methyl ester group, and so all such forms are considered to be disclosed herein with reference to the compounds listed above.

The prodrug form of the above compounds may be explicitly considered to include C₁-C₂₀ ester or ester comprising a cycloalkyl, or aryl moiety. The aryl moiety may include substituted phenyl or fused 2-3 cyclic aromatic rings.

In one embodiment, the compound of the first aspect is a haemagglutinin-neuraminidase modulator. That is, the compound of the first aspect is a modulator of haemagglutinin and/or neuraminidase functions. Preferably, the compound of the first aspect is a haemagglutinin-neuraminidase inhibitor. That is, an inhibitor of haemagglutinin and/or neuraminidase functions. This may include blocking of the haemagglutination function through modulation of the haemagglutinin protein.

In one embodiment, it may be preferred that the haemagglutinin-neuraminidase inhibitor is an influenza or parainfluenza haemagglutinin and/or neuraminidase inhibitor or blocker. Put another way, in one embodiment, it may be preferred that the inhibitor of haemagglutinin and/or neuraminidase functions is an inhibitor of influenza or parainfluenza haemagglutinin and/or neuraminidase functions. This may include blocking of the influenza or parainfluenza haemagglutination function and so modulation of the influenza haemagglutinin protein or parainfluenza haemagglutinin-neuraminidase protein.

A number of synthetic pathways can be employed to access the compounds of the invention. The experimental section details certain pathways by which certain inhibitors of the invention were synthesised to use as reference compounds. Relevant synthetic techniques, which may also be applied to synthesis of compounds of the first aspect, are disclosed in Nature Scientific Reports, 7:4507, 3 Jul. 2017; Angew. Chem. Int. Ed. 2015, 54, 2936-2940; Nature Scientific Reports, 6:24138, 7 Apr. 2016; Med. Chem. Commun., 2017, 8, 130-134; J. Med. Chem. 2014, 57, 7613-7623; Carbohydr. Res. 244, 181-185 (1993); Nature Communications, 5:5268, 20 Oct. 2014; Viruses, 2019, 11, 417, 05 May 2019; Carbohydr. Res. 342, 1636-1650 (2007); Bioorg. Med. Chem. Lett. 16, 5009-5013 (2006); PCT application WO2002076971; and PCT application WO2016033660, each of which is hereby incorporated by reference in their entirety. Such techniques and synthetic approaches can be employed to access all of the compounds of the first aspect.

According to a second aspect of the invention there is provided a pharmaceutical composition comprising an effective amount of a compound of any embodiment or formulae of the first aspect, or a pharmaceutically acceptable salt thereof, and a pharmaceutically acceptable carrier, diluent and/or excipient.

Suitably, the pharmaceutical composition is for the treatment or prophylaxis of a disease, disorder or condition caused by viral infection.

The pharmaceutical composition may include more than one compound of formula (I). When the composition includes more than one compound then the compounds may be in any ratio. The composition may further comprise known co-actives, delivery vehicles or adjuvants.

The compound of any embodiment or formulae of the first aspect is present in the pharmaceutical composition in an amount sufficient to inhibit or ameliorate the disease, disorder or condition which is the subject of treatment. Suitable dosage forms and rates of the compounds and the pharmaceutical compositions containing such may be readily determined by those skilled in the art.

Dosage forms may include tablets, dispersions, mists, aerosols, suspensions, injections, solutions, syrups, troches, capsules and the like.

A third aspect of the invention resides in a method of treating a disease, disorder or condition caused by viral infection in a patient including the step of administering an effective amount of a compound of any embodiment or formulae of the first aspect, or a pharmaceutically effective salt thereof, or the pharmaceutical composition of the second aspect to the patient.

A fourth aspect of the invention provides for a compound of any embodiment or formulae of the first aspect, or a pharmaceutically effective salt thereof, or the pharmaceutical composition of the second aspect for use in the treatment of a disease, disorder or condition caused by viral infection in a patient.

A fifth aspect of the invention provides for use of a compound of any embodiment or formulae of the first aspect, or a pharmaceutically effective salt thereof, in the manufacture of a medicament for the treatment of a disease, disorder or condition caused by viral infection.

In one embodiment of the third, fourth or fifth aspects, the disease, disorder or condition is an infection caused by an influenza and/or parainfluenza virus.

The infection may be caused by one or more of an influenza A virus, influenza B virus, influenza C virus, influenza D virus, parainfluenza virus, respiratory syncytial virus (RSV) and human metapneumovirus (hMPV).

When the disease, disorder or condition is parainfluenza viral infection, it may be selected from the group consisting of an hPIV-1, -2, -3 and -4 virus. These may include all viral subtypes, e.g. 4a and 4b.

When the disease, disorder or condition is caused by RSV then it may be the A and/or B subtypes, for example, hRSV-A and hRSV-B.

When the disease, disorder or condition is caused by hMPV then it may be caused by any one or more of the hMPV A1, A2, B1 and B2 subtypes

Preferably, the patient is a domestic or livestock animal or a human.

A sixth aspect of the invention provides for a method of modulating viral haemagglutinin and/or neuraminidase function including the step of contacting the viral haemagglutinin-neuraminidase with a compound of any embodiment or formulae of the first aspect.

Preferably, the modulating involves inhibiting the viral haemagglutinin and/or neuraminidase functions or viral haemagglutinin-neuraminidase enzyme.

The following experimental section describes in more detail the characterisation of certain of the compounds of the invention and their antiviral activity. The intention is to illustrate certain specific embodiments of the compounds of the invention and their efficacy without limiting the invention in any way.

### EXPERIMENTAL

### Chemistry

### General Methods

Reagents and dry solvents were purchased from commercial sources and used without further purification. Anhydrous reactions were carried out under an atmosphere of argon in oven-dried glassware. Reactions were monitored using thin layer chromatography (TLC) on aluminium plates pre-coated with Silica Gel 60 F254 (E. Merck). Developed plates were observed under UV light at 254 nm and then visualized after application of a solution of H₂SO₄ in EtOH (5% v/v) followed by charring. Flash chromatography was performed on Silica Gel 60 (0.040-0.063 mm) using distilled solvents. ¹H and ¹³C NMR spectra were recorded at 400 and 100 MHz respectively on a BrukerAvance 400 MHz spectrometer. Chemical shifts (6) are reported in parts per million, relative to the residual solvent peak as internal reference [CDCl₃: 7.26 (s) for ¹H, 77.0 (t) for ¹³C; CD₃OD: 4.78 (s) and 3.31 (pent) for ¹H, 49.15 (hept) for ¹³C; D₂O: 4.79 (s) for ¹H]. 2D COSY and HSQC experiments were run to support assignments. Low-resolution mass spectra (LRMS) were recorded, in electrospray ionization mode, on a BrukerDaltonics Esquire 3000 ESI spectrometer, using positive mode.

### Synthesis

### Naphthotriazole Synthesis

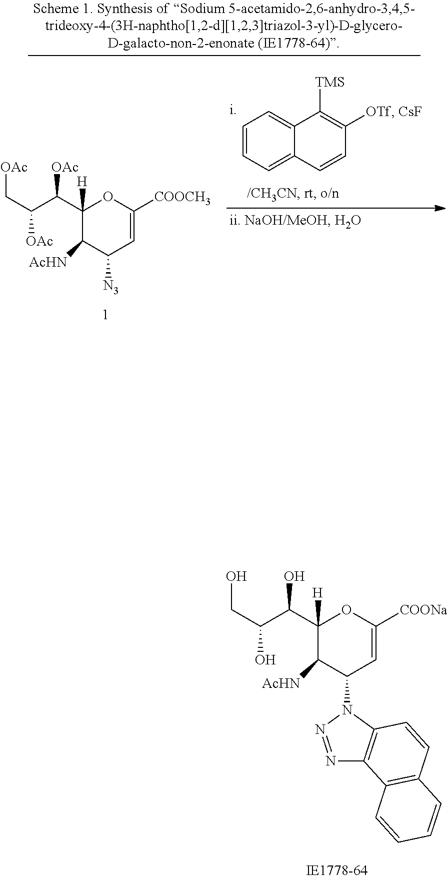

To a solution of the azide derivative 1 (60 mg, 0.13 mmol) in anhydrous acetonitrile (3 mL) was added cesium fluoride (40 mg, 0.26 mmol) followed by 1-(trimethylsilyl)-2-naphtyltrifluoromethane sulfonate (55 μL, 0.197 mmol), and the reaction mixture was stirred at rt under argon o/n. Sat. aq. NaHCO₃ (20 mL) was added, and the mixture was extracted with ethyl acetate (100 mL). The organic layer was separated, washed with water, brine, then dried over anhydrous Na₂SO₄. The dried organic solvent was concentrated under vacuum, and purified by silica gel chromatography using ethyl acetate:hexane (6:1) as solvent to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1778-64 (38 mg, 64% yield over two steps) as fluffy white powder after freeze-drying. ¹H NMR (400 MHz, D₂O): δ 1.62 (s, 3H), 3.63-3.76 (m, 2H), 3.93 (dd, J=12.1, 2.7 Hz, 1H), 4.07 (ddd, J=9.3, 6.3, 2.7 Hz, 1H), 4.58 (t, J=10.3 Hz, 1H), 4.67-4.74 (m, 1H), 5.91 (dd, J=9.7, 2.3 Hz, 1H), 6.12 (d, J=2.3 Hz, 1H), 7.60-7.73 (m, 2H), 7.74-7.83 (m, 1H), 7.90 (d, J=9.1 Hz, 1H), 8.03 (d, J=8.0 Hz, 1H), 8.51 (d, J=8.2 Hz, 1H); ¹³C NMR (101 MHz, D₂O): 21.44, 48.07, 59.01, 63.09, 68.10, 69.76, 75.39, 102.37, 109.52, 121.30, 124.00, 126.96, 128.50, 128.91, 130.26, 130.62, 131.42, 141.56, 150.48, 168.83, 173.22; LRMS [C₂₁H₂₁N₄NaO₇] (m/z): (+ve ion mode) 487.1 [M+Na]⁺

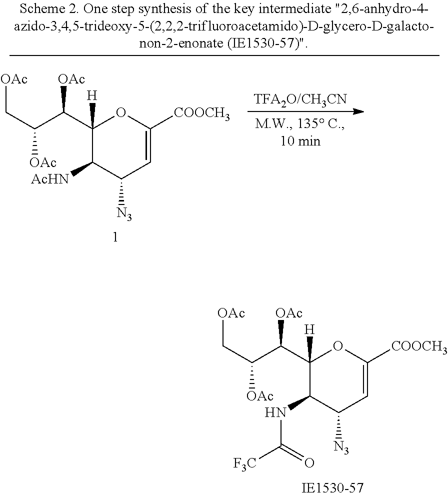 [SMILES: COC(=O)C1=C[C@H](N=[N+]=[N-])[C@@H](NC(C)=O)[C@H]([C@H](OC(C)=O)[C@@H](COC(C)=O)OC(C)=O)O1 ; COC(=O)C1=C[C@H](N=[N+]=[N-])[C@@H](NC(=O)C(F)(F)F)[C@H]([C@H](OC(C)=O)[C@@H](COC(C)=O)OC(C)=O)O1]

To a solution of the azide derivative 1 (100 mg, 0.22 mmol) in acetonitrile (0.5 mL) was added trifluoroacetic anhydride (220 μL, 1.54 mmol) and the mixture was heated in microwave reactor at 135° C. for 10 min. After cooling, MeOH (1 mL) was added, and the reaction mixture was concentrated under vacuum. The crude product was purified by silica gel chromatography using hexane:acetone (3:2) as solvent to yield pure IE1530-57 (84 mg, 75%). ¹H NMR (400 MHz, CDCl₃): δ 2.05 (s, 3H), 2.08 (s, 3H), 2.14 (s, 3H), 3.82 (s, 3H), 3.92 (q, J=8.9 Hz, 1H), 4.19 (dd, J=12.5, 6.5 Hz, 1H), 4.47-4.57 (m, 2H), 4.66 (dd, J=12.5, 2.7 Hz, 1H), 5.31 (td, J=6.0, 5.4, 2.8 Hz, 1H), 5.39 (dd, J=5.2, 2.3 Hz, 1H), 6.02 (d, J=2.7 Hz, 1H), 7.22 (d, J=8.8 Hz, 1H); ¹³C NMR (101 MHz, CDCl₃): δ 20.61, 20.66, 20.87, 49.05, 52.77, 57.03, 61.79, 67.54, 70.84, 75.01, 107.05, 115.39 (q, J=288.0 Hz), 145.25, 157.66 (d, J=38.3 Hz), 161.25, 170.50, 170.65, 170.92; LRMS [C₁₈H₂₁F₃N₄O₁₀] (m/z): (+ve ion mode) 533.2 [M+Na]⁺

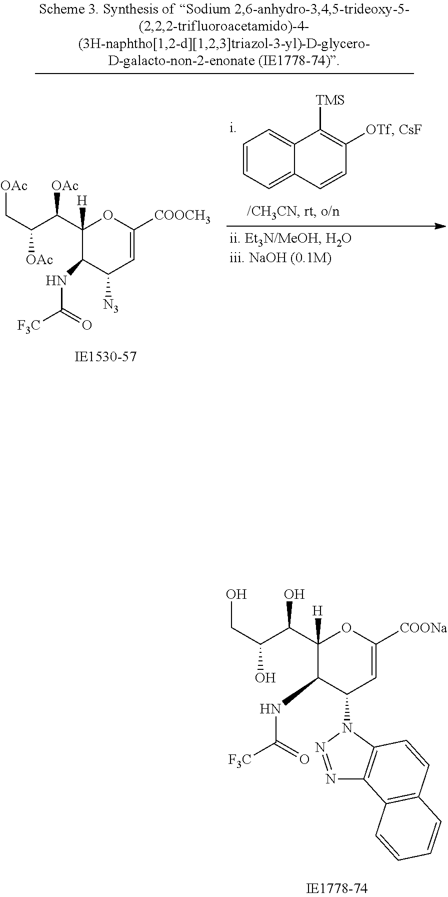

To a solution of the azide derivative IE1530-57 (60 mg, 0.118 mmol) in anhydrous acetonitrile (3 mL) was added cesium fluoride (36 mg, 0.24 mmol) followed by 1-(trimethylsilyl)-2-naphtyltrifluoromethane sulfonate (50 μL, 0.177 mmol), and the reaction mixture was stirred at rt under argon o/n. Sat. aq. NaHCO₃ (20 mL) was added, and the mixture was extracted with ethyl acetate (100 mL). The organic layer was separated, washed with water, brine, then dried over anhydrous Na₂SO₄. The dried organic solvent was concentrated under vacuum, and purified by silica gel chromatography using hexane:acetone (2:1) as solvent to yield pure protected product. The protected product was suspended in a 1:1 mixture of MeOH:H₂O (2 mL). To this suspension at 0° C. was added dropwise a triethylamine (1 mL). The temperature was gradually raised to rt and the mixture was stirred at rt overnight. The solution was concentrated under vacuum and the pH was adjusted to 8.0 using 0.05 M NaOH to convert the compound to its sodium salt. Finally, the compound was purified on a C18-GracePure™ cartridge using 2% acetonitrile/water as solvent to yield the pure deprotected product IE1778-74 (36 mg, 59% yield over two steps) as fluffy yellowish powder after freeze-drying. ¹H NMR (400 MHz, D₂O): δ 3.63-3.75 (m, 2H), 3.93 (dd, J=12.0, 2.7 Hz, 1H), 4.08 (ddd, J=9.4, 6.5, 2.7 Hz, 1H), 4.66-4.73 (m, 1H), 4.75 (s, 1H), 5.99 (dd, J=9.5, 2.3 Hz, 1H), 6.14 (d, J=2.2 Hz, 1H), 7.67-7.77 (m, 2H), 7.81 (ddd, J=8.2, 7.1, 1.2 Hz, 1H), 7.95 (d, J=9.2 Hz, 1H), 8.07 (d, J=8.0 Hz, 1H), 8.53-8.60 (m, 1H); ¹³C NMR (101 MHz, D₂O): δ 49.49, 58.86, 58.95, 63.10, 68.27, 69.78, 75.56, 102.41, 109.70, 121.34, 124.01, 127.03, 128.54, 128.97, 130.33, 130.74, 131.46, 141.63, 150.56, 168.77; LRMS [C₂₁H₁₈F₃N₄NaO₇] (m/z): (+ve ion mode) 541.1 [M+Na]⁺

### Indazole Synthesis

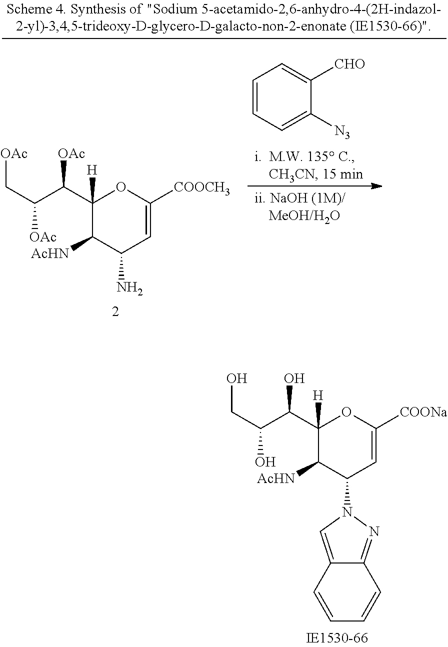

To a solution of the amine derivative 2 (60 mg, 0.14 mmol) in anhydrous acetonitrile (1 mL) was added 2-azidobenzaldehyde (30 mg, 0.21 mmol) and the reaction mixture was heated at 135° C. under Microwave irradiation for 15 min. The mixture was left to cool to rt, concentrated under vacuum, and purified by silica gel chromatography using ethyl acetate:hexane (4:1) as solvent to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1530-66 (33 mg, 58% yield over two steps) as fluffy powder after freeze-drying. 1H NMR (400 MHz, D₂O): δ 1.83 (s, 3H), 3.63-3.73 (m, 2H), 3.92 (dd, J=12.0, 2.7 Hz, 1H), 4.03 (ddd, J=9.3, 6.3, 2.7 Hz, 1H), 4.47-4.61 (m, 2H), 5.40-5.47 (m, 1H), 5.93 (d, J=2.2 Hz, 1H), 7.18 (ddd, J=8.5, 6.6, 0.9 Hz, 1H), 7.41 (ddd, J=8.8, 6.7, 1.1 Hz, 1H), 7.67 (d, J=8.8 Hz, 1H), 7.79 (d, J=8.5 Hz, 1H), 8.35 (s, 1H); ¹³C NMR (101 MHz, D₂O): 21.61, 48.98, 61.78, 63.10, 68.18, 69.78, 75.39, 103.54, 116.02, 120.94, 122.01, 124.70, 127.28, 148.42, 149.77, 169.08, 173.46; LRMS [C₁₈H₂₀N₃NaO₇](m/z): (+ve ion mode) 436.2 [M+Na]⁺

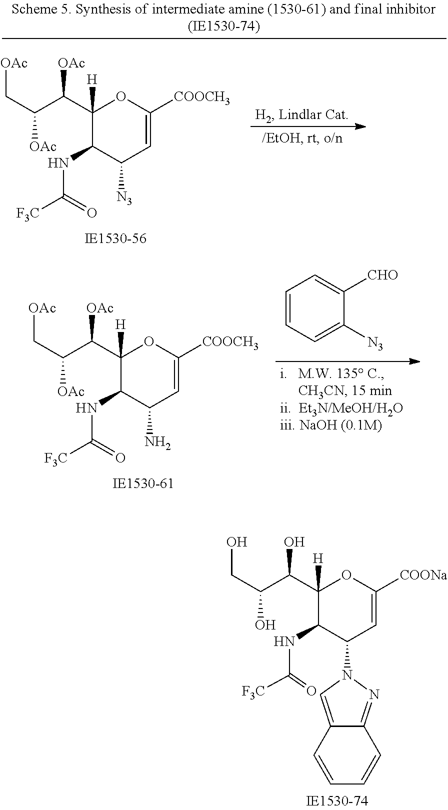

### Methyl 7,8,9-Tri-O-acetyl-4-amino-2,6-anhydro-3,4,5-trideoxy-5-(2,2,2-trifluoroacetamido)-D-glycero-D-galacto-non-2-enonate (IE1530-61)

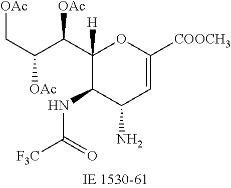 [SMILES: COC(=O)C1=C[C@H](N)[C@@H](NC(=O)C(F)(F)F)[C@H]([C@H](OC(C)=O)[C@@H](COC(C)=O)OC(C)=O)O1]

To a solution of the azide derivative IE1530-56 (200 mg, 0.41 mmol) in ethanol (5 mL) was added Lindlar catalyst (20 mg) and the reaction mixture was stirred under H₂ atmosphere at rt o/n. Upon reaction completion, the reaction mixture was filtered through celite bed, followed by washing with ethanol (50 mL). The combined filtrate and washing were combined and concentrated under vacuum to yield crude IE1530-61 (quantitative yield) that was of good purity to be used in next step without further purification. LRMS [C₁₈H₂₆N₂O₁₀] (m/z): (+ve ion mode) 507.2 [M+Na]⁺

### Sodium 2,6-anhydro-3,4,5-trideoxy-4-(2H-indazol-2-yl)-5-(2,2,2-trifluoroacetamido)-D-glycero-D-galacto-non-2-enonate (IE1530-74)

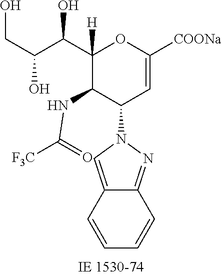

To a solution of the amine derivative IE1530-61 (50 mg, 0.103 mmol) in anhydrous acetonitrile (1 mL) was added 2-azidobenzaldehyde (22 mg, 0.15 mmol) and the reaction mixture was heated at 135° C. under Microwave irradiation for 15 min. The mixture was left to cool to rt, concentrated under vacuum, and purified by silica gel chromatography using ethyl acetate:hexane (3:1) as solvent to yield pure protected product. The protected product was suspended in a 1:1 mixture of MeOH:H₂O (2 mL). To this suspension at 0° C. was added dropwise a triethylamine (1 mL). The temperature was gradually raised to rt and the mixture was stirred at rt overnight. The solution was concentrated under vacuum and the pH was adjusted to 8.0 using 0.05 M NaOH to convert the compound to its sodium salt. Finally, the compound was purified on a C18-GracePure™ cartridge using 2% acetonitrile/water as solvent to yield the pure deprotected product IE1530-74 (26 mg, 55% yield over two steps). ¹H NMR (400 MHz, D₂O): δ 3.63-3.73 (m, 2H), 3.92 (dd, J=12.0, 2.7 Hz, 1H), 4.05 (ddd, J=9.4, 6.4, 2.7 Hz, 1H), 4.58-4.77 (m, 2H), 5.54 (dd, J=9.4, 2.3 Hz, 1H), 5.99 (d, J=2.2 Hz, 1H), 7.19 (ddd, J=8.5, 6.7, 0.9 Hz, 1H), 7.42 (ddd, J=8.8, 6.6, 1.1 Hz, 1H), 7.66 (dd, J=8.8, 1.0 Hz, 1H), 7.79 (d, J=8.5 Hz, 1H), 8.39 (d, J=1.0 Hz, 1H); ¹³C NMR (101 MHz, D₂O): 49.79, 61.29, 63.05, 68.21, 69.73, 74.93, 103.18, 111.19-119.63 (m), 120.94, 121.46, 122.16, 124.69, 127.44, 148.57, 150.06, 158.32 (q, J=38.0, 37.1 Hz), 168.85; LRMS [C₁₈H₁₇F₃N₃NaO₇] (m/z): (+ve ion mode) 490.2 [M+Na]⁺

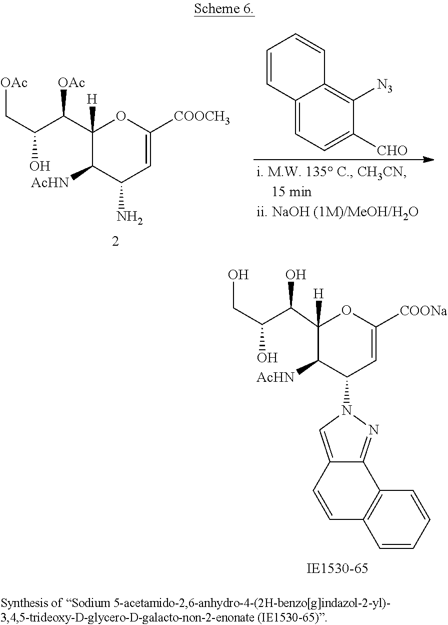

To a solution of the amine derivative 2 (60 mg, 0.14 mmol) in anhydrous acetonitrile (1 mL) was added 1-azido-2-naphthaldehyde (41 mg, 0.21 mmol) and the reaction mixture was heated at 135° C. under Microwave irradiation for 15 min. The mixture was left to cool to rt, concentrated under vacuum, and purified by silica gel chromatography using ethyl acetate:hexane (3:1) as solvent to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1530-65 (36 mg, 56% yield over two steps) as fluffy powder after freeze-drying. ¹H NMR (400 MHz, D₂O): δ 1.81 (s, 3H), 3.63-3.75 (m, 2H), 3.93 (dd, J=12.0, 2.7 Hz, 1H), 4.04 (ddd, J=9.3, 6.3, 2.7 Hz, 1H), 4.51 (t, J=10.2 Hz, 1H), 4.56-4.64 (m, 1H), 5.43 (dd, J=9.5, 2.3 Hz, 1H), 5.97 (d, J=2.2 Hz, 1H), 7.43 (d, J=9.0 Hz, 1H), 7.56-7.69 (m, 3H), 7.86-7.95 (m, 1H), 8.27 (s, 1H), 8.43-8.50 (m, 1H); ¹³C NMR (101 MHz, D₂O): δ 21.62, 49.09, 61.54, 63.11, 68.19, 69.79, 75.48, 103.51, 118.69, 118.89, 121.74, 123.62, 124.05, 125.21, 126.93, 127.28, 128.65, 132.49, 145.82, 149.94, 169.13, 173.48; LRMS [C₂₂H₂₂N₃NaO₇] (m/z): (+ve ion mode) 486.2 [M+Na]⁺

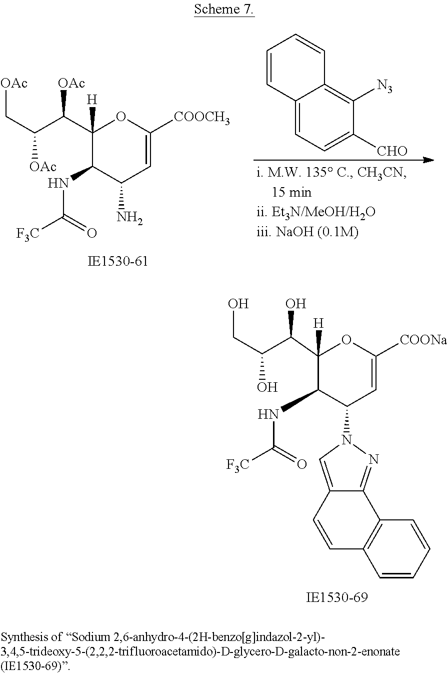

To a solution of the amine derivative IE1530-61 (50 mg, 0.103 mmol) in anhydrous acetonitrile (1 mL) was added 1-azido-2-naphthaldehyde (30 mg, 0.15 mmol) and the reaction mixture was heated at 135° C. under Microwave irradiation for 15 min. The mixture was left to cool to rt, concentrated under vacuum, and purified by silica gel chromatography using ethyl acetate:hexane (2:1) as solvent to yield pure protected product. The protected product was suspended in a 1:1 mixture of MeOH:H₂O (2 mL). To this suspension at 0° C. was added dropwise a triethylamine (1 mL). The temperature was gradually raised to rt and the mixture was stirred at rt overnight. The solution was concentrated under vacuum and the pH was adjusted to 8.0 using 0.05 M NaOH to convert the compound to its sodium salt. Finally, the compound was purified on a C18-GracePure™ cartridge using 2% acetonitrile/water as solvent to yield the pure deprotected product IE1530-69 (28 mg, 59% yield over two steps). ¹H NMR (400 MHz, D₂O): δ 3.64-3.74 (m, 2H), 3.93 (dd, J=11.9, 2.7 Hz, 1H), 4.06 (ddd, J=9.4, 6.4, 2.7 Hz, 1H), 4.59 (t, J=10.2 Hz, 1H), 4.69-4.76 (m, 1H), 5.52 (d, J=8.8 Hz, 1H), 6.04 (d, J=2.2 Hz, 1H), 7.45 (d, J=9.1 Hz, 1H), 7.63 (dd, J=19.3, 8.1 Hz, 3H), 7.91 (d, J=7.2 Hz, 1H), 8.31 (s, 1H), 8.45 (d, J=8.2 Hz, 1H); ¹³C NMR (101 MHz, D₂O): δ 50.17, 61.11, 63.08, 68.24, 69.75, 75.09, 115.53 (q, J=286.1, 285.1 Hz), 118.83, 121.84, 123.79, 123.99, 125.03, 126.94, 127.36, 128.64, 132.54, 145.98, 150.30, 158.37 (d, J=37.7 Hz), 168.92; LRMS [C₂₂H₁₉F₃N₃NaO₇] (m/z): (+ve ion mode) 540.2 [M+Na]⁺

### Alternate Indazole Synthesis

The previously described method for the synthesis of indazoles included heating the amine with 2-azido-1-carboxaldehyde derivatives at high temperature in Microwave reactor to affect imine-formation and cyclization in a single step. It was found that this method cannot be used with all 2-azido-1-carboxaldehyde derivatives. Accordingly, the copper catalysed one-pot synthesis shown below was developed:

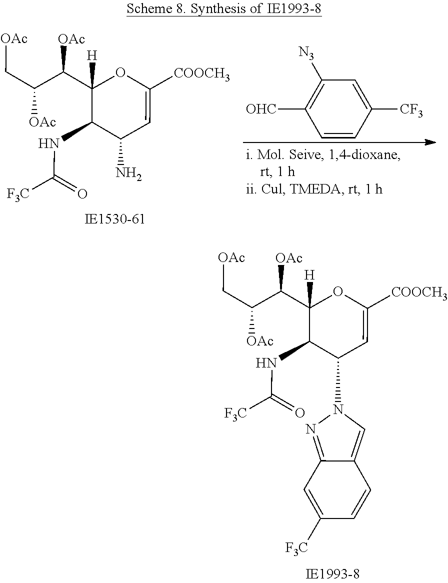 [SMILES: COC(=O)C1=C[C@H](N)[C@@H](NC(=O)C(F)(F)F)[C@H]([C@H](OC(C)=O)[C@@H](COC(C)=O)OC(C)=O)O1 ; [N-]=[N+]=Nc1cc(C(F)(F)F)ccc1C=O ; COC(=O)C1=C[C@H](n2cc3ccc(C(F)(F)F)cc3n2)[C@@H](NC(=O)C(F)(F)F)[C@H]([C@H](OC(C)=O)[C@@H](COC(C)=O)OC(C)=O)O1]

### Experimental Procedure

To a mixture of amine (50 mg, 0.103 mM), 2-azido-1-carboxaldehyde derivative (1.2 eq), CuI (0.1 eq), and 4A Molecular Sieve (50 mg) in dry 1,4-dioxane (2 mL) under argon, was added tetramethylethylenediamine (TMEDA, 1.0 eq). The mixture was allowed to stir at rt for 1 h, and was then filtered over celite, concentrated and purified by flash silica column chromatography.

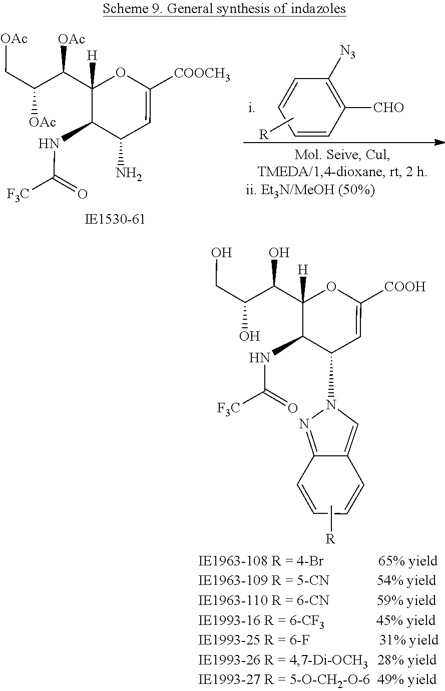

### General Synthesis of the Indazoles (Scheme 9)

To a mixture of amine (1.0 eq), 2-azido-1-carboxaldehyde derivative (1.2 eq), CuI (0.1 eq), and 4A Molecular Sieve (50 mg) in dry 1,4-dioxane (2 mL) under argon, was added tetramethylethylene-diamine (TMEDA, 1.0 eq). The mixture was allowed to stir at rt for 1 h, and was then filtered over celite, concentrated and purified by flash silica column chromatography to yield the protected indazole product. The protected product was suspended in a 1:1 mixture of MeOH:H₂O (2 mL). To this suspension at 0° C. was added dropwise Et₃N (1.0 mL) and the temperature was gradually raised to rt and the mixture was stirred at rt overnight. The solution was concentrated under vacuum, then purified by flash silica column chromatography to yield the pure deprotected product.

### IE1963-108

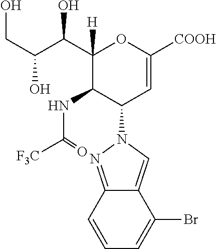 [SMILES: O=C(O)C1=C[C@H](n2cc3c(Br)cccc3n2)[C@@H](NC(=O)C(F)(F)F)[C@H]([C@H](O)[C@H](O)CO)O1]

¹H NMR (400 MHz, CD₃OD): δ 3.56 (d, J=9.4 Hz, 1H), 3.68 (dd, J=11.5, 5.5 Hz, 1H), 3.84 (dd, J=11.5, 2.9 Hz, 1H), 3.95 (ddd, J=9.4, 5.4, 2.9 Hz, 1H), 4.63-4.72 (m, 2H), 5.57 (td, J=5.0, 2.2 Hz, 1H), 5.86 (d, J=2.1 Hz, 1H), 7.16 (dd, J=8.6, 7.2 Hz, 1H), 7.25 (d, J=7.1 Hz, 1H), 7.54-7.59 (m, 1H), 8.28 (d, J=0.9 Hz, 1H); ¹³C NMR (101 MHz, CD₃OD): δ 49.78, 61.34, 63.39, 68.69, 69.92, 75.03, 101.18, 112.68, 115.73 (q, J=287.2 Hz), 116.05, 123.53, 123.60, 124.12, 126.76, 148.69, 151.06, 157.34 (q, J=37.6 Hz), 167.81; LRMS [C₁₈H₁₇BrF₃N₃O₇] (m/z): (+ve ion mode) 569.4 [M+Na]⁺.

### IE1963-109

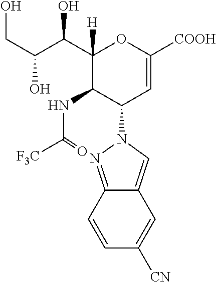 [SMILES: N#Cc1ccc2nn([C@H]3C=C(C(=O)O)O[C@@H]([C@H](O)[C@H](O)CO)[C@@H]3NC(=O)C(F)(F)F)cc2c1]

¹H NMR (400 MHz, CD₃OD): δ 3.56 (d, J=9.4 Hz, 1H), 3.68 (dd, J=11.5, 5.4 Hz, 1H), 3.84 (dd, J=11.5, 2.9 Hz, 1H), 3.95 (ddd, J=9.5, 5.4, 2.9 Hz, 1H), 4.64-4.73 (m, 2H), 5.56-5.65 (m, 1H), 5.84 (d, J=2.1 Hz, 1H), 7.42 (dd, J=9.0, 1.5 Hz, 1H), 7.72 (dt, J=9.0, 1.0 Hz, 1H), 8.27 (t, J=1.2 Hz, 1H), 8.54 (d, J=0.9 Hz, 1H); ¹³C NMR (101 MHz, CD₃OD): δ 49.80, 61.70, 63.38, 68.64, 69.89, 75.04, 100.91, 104.58, 115.73 (q, J=287.2 Hz), 118.37, 119.30, 121.02, 125.13, 126.40, 128.75, 148.60, 151.24, 157.33 (d, J=37.4 Hz), 167.69; LRMS [C₁₉H₁₇F₃N₄O₇] (m/z): (+ve ion mode) 514.5 [M+Na]⁺.

### IE1963-110

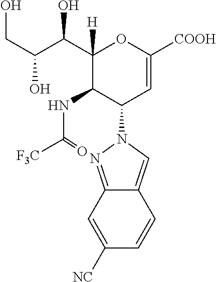 [SMILES: N#Cc1ccc2cn([C@H]3C=C(C(=O)O)O[C@@H]([C@H](O)[C@H](O)CO)[C@@H]3NC(=O)C(F)(F)F)nc2c1]

¹H NMR (400 MHz, CD₃OD): δ 3.56 (d, J=9.5 Hz, 1H), 3.67 (dd, J=11.5, 5.5 Hz, 1H), 3.84 (dd, J=11.5, 2.9 Hz, 1H), 3.95 (ddd, J=9.5, 5.5, 2.8 Hz, 1H), 4.65-4.72 (m, 2H), 5.62 (td, J=5.0, 2.2 Hz, 1H), 5.84 (d, J=2.1 Hz, 1H), 7.23 (dd, J=8.7, 1.3 Hz, 1H), 7.87 (dd, J=8.7, 1.0 Hz, 1H), 8.10 (d, J=1.1 Hz, 1H), 8.45 (d, J=1.0 Hz, 1H); ¹³C NMR (101 MHz, CD₃OD): δ 49.83, 61.81, 63.41, 68.68, 69.88, 75.06, 101.04, 109.07, 115.70 (d, J=287.1 Hz), 119.03, 121.70, 122.45, 123.36, 123.79, 123.96, 146.84, 151.14, 157.28 (q, J=37.3 Hz), 167.60; LRMS [C₁₉H₁₇F₃N₄O₇] (m/z): (+ve ion mode) 514.5 [M+Na]⁺.

### IE1993-25

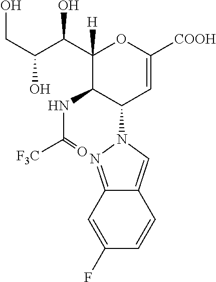 [SMILES: O=C(O)C1=C[C@H](n2cc3ccc(F)cc3n2)[C@@H](NC(=O)C(F)(F)F)[C@H]([C@H](O)[C@H](O)CO)O1]

¹H NMR (400 MHz, CD₃OD): δ 3.54 (d, J=9.5 Hz, 1H), 3.67 (dd, J=11.5, 5.5 Hz, 1H), 3.84 (dd, J=11.5, 2.9 Hz, 1H), 3.94 (ddd, J=9.0, 5.5, 2.8 Hz, 1H), 4.64-4.70 (m, 2H), 5.49-5.56 (m, 1H), 5.82 (d, J=2.2 Hz, 1H), 6.79 (dd, J=8.9, 1.9 Hz, 1H), 7.18-7.24 (m, 1H), 7.71 (dd, J=8.8, 0.8 Hz, 1H), 8.27 (s, 1H); ¹³C NMR (101 MHz, CD₃OD): δ 49.69, 61.15, 63.45, 68.75, 69.88, 75.00, 101.34, 104.22, 113.78 (d, J=104.8 Hz), 115.37, 119.85, 122.26, 123.39, 138.61, 148.82, 150.99, 157.25 (d, J=37.5 Hz), 167.83; LRMS [C₁₈H₁₇F₄N₃O₇] (m/z): (+ve ion mode) 484.0 [M+Na]⁺.

### IE1993-26

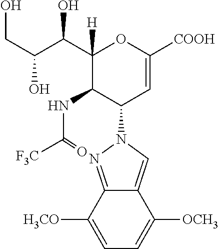 [SMILES: COc1ccc(OC)c2nn([C@H]3C=C(C(=O)O)O[C@@H]([C@H](O)[C@H](O)CO)[C@@H]3NC(=O)C(F)(F)F)cc12]

¹H NMR (400 MHz, CD₃OD): δ 3.56 (d, J=9.3 Hz, 1H), 3.67 (dd, J=11.5, 5.5 Hz, 1H), 3.80-3.89 (m, 4H), 3.9-3.97 (m, 4H), 4.62-4.75 (m, 2H), 5.45-5.55 (m, 1H), 5.80 (d, J=2.2 Hz, 1H), 6.24 (d, J=8.0 Hz, 1H), 6.50 (d, J=8.0 Hz, 1H), 8.19 (s, 1H); ¹³C NMR (101 MHz, CD₃OD): δ 49.56, 54.38, 54.73, 61.01, 63.43, 68.70, 70.00, 75.11, 97.86, 101.68, 103.45, 114.30, 116.98, 117.16, 121.09, 142.82, 144.02, 147.25, 150.75, 157.14; LRMS [C₂₀H₂₂F₃N₃O₉] (m/z): (−ve ion mode) 503.9 [M−H]⁺.

### IE1993-27

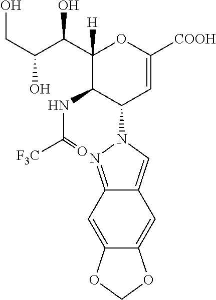 [SMILES: O=C(O)C1=C[C@H](n2cc3cc4c(cc3n2)OCO4)[C@@H](NC(=O)C(F)(F)F)[C@H]([C@H](O)[C@H](O)CO)O1]

¹H NMR (400 MHz, CD₃OD): δ 3.53 (d, J=9.4 Hz, 1H), 3.67 (dd, J=11.5, 5.5 Hz, 1H), 3.84 (dd, J=11.6, 2.9 Hz, 1H), 3.94 (ddd, J=9.1, 5.6, 2.8 Hz, 1H), 4.58-4.68 (m, 2H), 5.40 (d, J=7.1 Hz, 1H), 5.75-5.84 (m, 1H), 5.92 (s, 2H), 6.81 (s, 1H), 6.88 (s, 1H), 7.98 (s, 1H); ¹³C NMR (101 MHz, CD₃OD): δ 49.55, 60.58, 63.44, 68.75, 69.93, 75.01, 92.69, 94.79, 100.92, 101.89, 114.33, 117.31, 122.18, 145.71, 146.07, 149.63, 150.70, 157.24 (d, J=37.1 Hz); LRMS [C₁₉H₁₈F₃N₃O₉] (m/z): (−ve ion mode) 487.9 [M−H]⁺.

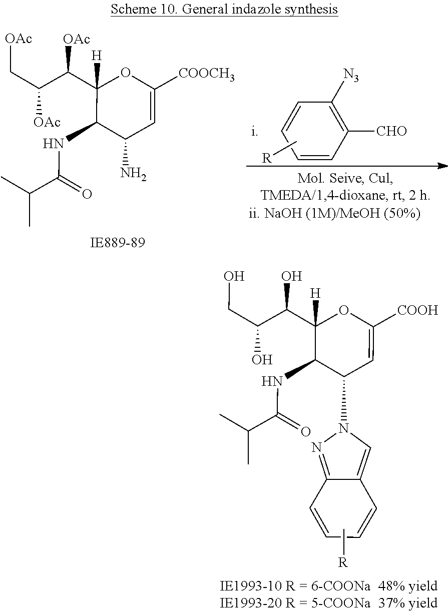

### General Synthesis of the Indazoles (Scheme 10)

To a mixture of amine (1.0 eq), 2-azido-1-carboxaldehyde derivative (1.2 eq), CuI (0.1 eq), and 4A Molecular Sieve (50 mg) in dry 1,4-dioxane (2 mL) under argon, was added tetramethylethylene-diamine (TMEDA, 1.0 eq). The mixture was allowed to stir at rt for 1 h, and was then filtered over celite, concentrated and purified by flash silica column chromatography to yield the protected indazole product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 10% methanol/water as solvent to yield the pure deprotected product.

### IE1993-10

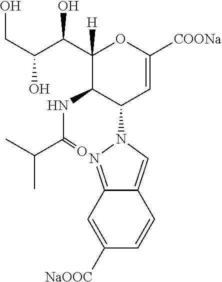

¹H NMR (400 MHz, D₂O): δ 0.83 (d, J=6.9 Hz, 3H), 0.93 (d, J=6.9 Hz, 3H), 2.37 (p, J=6.9 Hz, 1H), 3.63-3.71 (m, 2H), 3.93 (dd, J=12.0, 2.7 Hz, 1H), 4.03 (ddd, J=9.3, 6.4, 2.7 Hz, 1H), 4.55-4.66 (m, 2H), 5.48-5.54 (m, 1H), 5.95 (d, J=2.2 Hz, 1H), 7.60 (dd, J=8.7, 1.4 Hz, 1H), 7.80 (dd, J=8.8, 1.0 Hz, 1H), 8.15 (d, J=1.1 Hz, 1H), 8.39 (d, J=1.0 Hz, 1H); ¹³C NMR (101 MHz, D₂O): δ 18.39, 18.49, 35.02, 48.64, 62.00, 63.10, 68.26, 69.83, 75.41, 103.43, 117.57, 120.59, 121.99, 122.60, 124.69, 135.35, 147.94, 149.75, 169.08, 175.72, 180.56; LRMS [C₂₁H₂₃N₃Na₂O₉] (m/z): (+ve ion mode) 529.2 [M+Na]⁺.

### IE1993-20

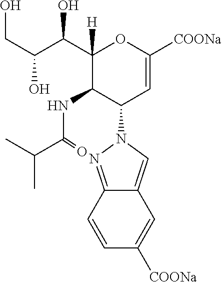

¹H NMR (400 MHz, D₂O): δ 0.84 (d, J=6.9 Hz, 3H), 0.93 (d, J=6.9 Hz, 3H), 2.38 (p, J=6.9 Hz, 1H), 3.63-3.71 (m, 2H), 3.92 (dd, J=12.0, 2.7 Hz, 1H), 4.03 (ddd, J=9.3, 6.4, 2.7 Hz, 1H), 4.53-4.65 (m, 2H), 5.51 (dd, J=9.2, 2.3 Hz, 1H), 5.95 (d, J=2.1 Hz, 1H), 7.65 (dt, J=9.2, 1.0 Hz, 1H), 7.85 (dd, J=9.1, 1.6 Hz, 1H), 8.34 (dd, J=1.6, 0.9 Hz, 1H), 8.50 (d, J=1.0 Hz, 1H).

### Phenyltriazole Derivative Synthesis

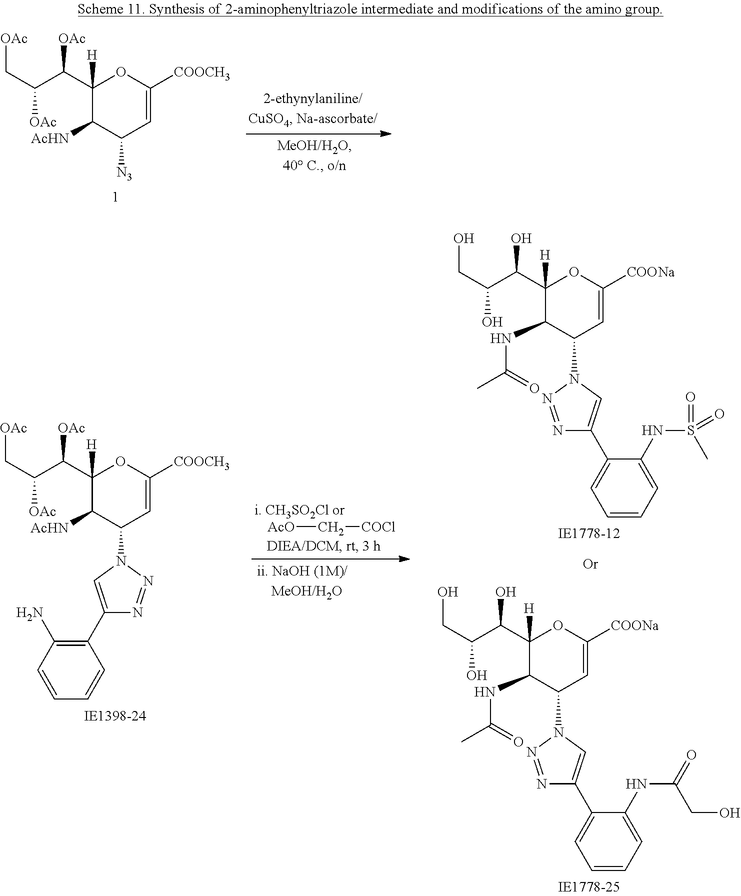

### Methyl 5-acetamido-7,8,9-tri-O-acetyl-4-((2-aminophenyl)-1H-1,2,3-triazol-1-yl)-2,6-anhydro-3,4,5-trideoxy-D-glycero-D-galacto-non-2-enonate (IE1398-24)

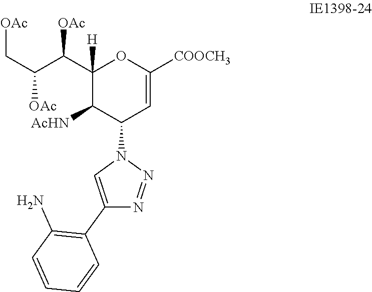 [SMILES: COC(=O)C1=C[C@H](n2cc(-c3ccccc3N)nn2)[C@@H](NC(C)=O)[C@H]([C@H](OC(C)=O)[C@@H](COC(C)=O)OC(C)=O)O1]

The 9-azido derivative 1 (500 mg, 1.1 mmol) and 1-amino-2-ethynylbenzene (140 μL, 1.2 mmol) were dissolved in a 4:1 mixture of MeOH:H₂O (4 mL). Copper(II)sulfate pentahydrate (50 mg, 0.22 mmol) was added to the mixture followed by sodium ascorbate (1.0 mL of freshly prepared 1 M solution in H₂O). The mixture was stirred at 45° C. o/n and then left to cool to rt. The mixture was then diluted with DCM (200 mL), washed with 10% NH₄OH (100 mL), followed by brine (100 mL). The organic layer was dried over anhydrous Na₂SO₄ and concentrated under vacuum to give the crude protected product, which was purified by silica gel chromatography using ethyl acetate:hexane (3:1) as solvent to yield pure IE1398-24 (484 mg, 0.84 mmol) at 77% yield. ¹H NMR (400 MHz, CDCl₃): δ 1.70 (s, 3H), 2.04 (s, 3H), 2.05 (s, 3H), 2.07 (s, 3H), 3.80 (s, 3H), 4.19 (dd, J=12.4, 7.4 Hz, 1H), 4.37 (q, J=10.0 Hz, 1H), 4.67 (dd, J=10.8, 1.9 Hz, 1H), 4.73 (dd, J=12.5, 2.6 Hz, 1H), 5.35 (ddd, J=7.5, 4.7, 2.6 Hz, 1H), 5.53 (dd, J=4.6, 1.8 Hz, 1H), 5.67 (dd, J=10.1, 2.4 Hz, 1H), 6.03 (d, J=2.3 Hz, 1H), 6.65-6.76 (m, 2H), 6.97 (d, J=9.2 Hz, 1H), 7.08 (ddd, J=8.3, 7.2, 1.6 Hz, 1H), 7.30 (dd, J=7.7, 1.5 Hz, 1H), 7.84 (s, 1H); ¹³C NMR (101 MHz, CDCl₃): δ 20.69, 20.82, 20.95, 22.72, 48.09, 52.71, 58.40, 62.22, 67.85, 71.39, 76.90, 107.36, 113.54, 116.72, 117.78, 119.35, 128.28, 129.43, 144.82, 145.75, 148.14, 161.32, 170.13, 170.51, 170.76, 171.05; LRMS [C₂₆H₃₁NO₁₀] (m/z): (+ve ion mode) 596.2 [M+Na]⁺

### Sodium 5-acetamido-2,6-anhydro-3,4,5-trideoxy-4-(2-(methylsulfonamido)phenyl)-1H-1,2,3-triazole-1-yl)-D-glycero-D-galacto-non-2-enonate (IE1778-12)

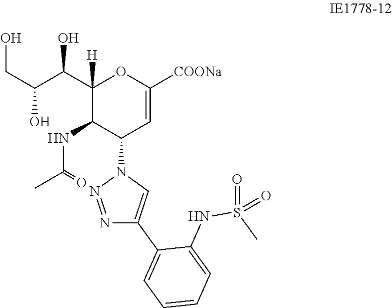

To a solution of IE1398-24 (50 mg, 0.087 mmol) in anhydrous dichloromethane (3 mL) was added diisopropylethylamine (46 μL, 0.26 mmol) followed by methylsulfonyl chloride (7.0 μL, 0.096 mmol) The mixture was stirred at rt for 3h, concentrated under vacuum to give the crude product, which was purified by silica gel chromatography using ethyl acetate:hexane (3:1) as solvent to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1778-12 (28 mg, 60% yield over two steps) as fluffy powder after freeze-drying. ¹H NMR (400 MHz, D₂O): δ 1.93 (s, 3H), 2.84 (s, 3H), 3.64-3.76 (m, 2H), 3.93 (dd, J=12.0, 2.7 Hz, 1H), 4.03 (ddd, J=9.3, 6.3, 2.6 Hz, 1H), 4.50 (dd, J=10.9, 9.5 Hz, 1H), 4.57-4.64 (m, 1H), 5.59 (dd, J=9.7, 2.3 Hz, 1H), 5.95 (d, J=2.2 Hz, 1H), 7.12 (ddd, J=7.8, 5.7, 2.8 Hz, 1H), 7.28-7.42 (m, 2H), 7.95 (dt, J=7.7, 1.3 Hz, 1H), 8.66 (s, 1H); ¹³C NMR (101 MHz, D₂O): δ 21.77, 39.74, 48.72, 59.61, 63.11, 68.16, 69.76, 75.42, 102.39, 121.36, 123.73, 123.97, 123.99, 127.76, 129.27, 143.92, 145.41, 150.14, 168.86, 173.75; LRMS [C₂₀H₂₄N₅NaO₉S] (m/z): (+ve ion mode) 556.1 [M+Na]⁺

### Sodium 5-acetamido-2,6-anhydro-3,4,5-trideoxy-4-(2-(2-hydroxyacetamido)phenyl)-1H-1,2,3-triazol-1-yl)-D-glycero-D-galacto-non-2-enonate (IE1778-25)

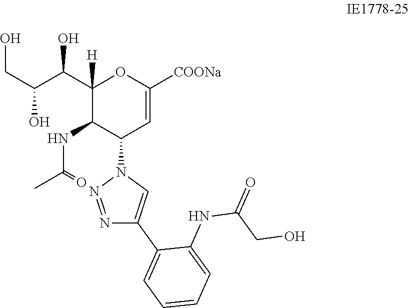

To a solution of IE1398-24 (50 mg, 0.087 mmol) in anhydrous dichloromethane (3 mL) was added diisopropylethylamine (46 μL, 0.26 mmol) followed by acetoxyacetyl chloride (10 μL, 0.096 mmol) The mixture was stirred at rt for 3h, concentrated under vacuum to give the crude product, which was purified by silica gel chromatography using ethyl acetate:hexane (4:1) as solvent to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1778-25 (33 mg, 74% yield over two steps) as fluffy powder after freeze-drying. ¹H NMR (400 MHz, D₂O): δ 1.93 (s, 3H), 3.63-3.77 (m, 2H), 3.93 (dd, J=12.0, 2.7 Hz, 1H), 4.03 (ddd, J=9.3, 6.3, 2.7 Hz, 1H), 4.22 (s, 2H), 4.47 (dd, J=10.9, 9.6 Hz, 1H), 4.60 (dd, J=10.9, 1.3 Hz, 1H), 5.61 (dd, J=9.6, 2.3 Hz, 1H), 5.89 (d, J=2.2 Hz, 1H), 7.45 (td, J=7.6, 1.4 Hz, 1H), 7.53 (td, J=7.8, 1.7 Hz, 1H), 7.76 (ddd, J=14.6, 7.8, 1.4 Hz, 2H), 8.33 (s, 1H); ¹³C NMR (101 MHz, D₂O): δ 21.68, 48.73, 60.06, 61.33, 63.08, 68.07, 69.72, 75.39, 101.85, 122.10, 123.92, 125.54, 127.13, 129.18, 129.63, 132.87, 145.31, 150.53, 168.75, 173.65, 174.10; LRMS [C₂₁H₂₄N₅NaO₉] (m/z): (+ve ion mode) 536.2 [M+Na]⁺

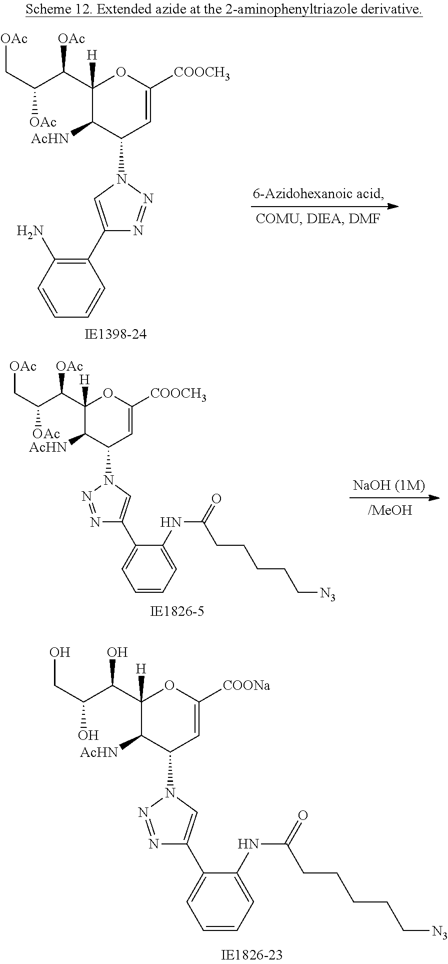

### Methyl 5-acetamido-7,8,9-tri-O-acetyl-2,6-anhydro-4-(2-(6-azidohexanamido)phenyl)-1H-1,2,3-triazol-1-yl)-3,4,5-trideoxy-D-glycero-D-galacto-non-2-enonate (IE1826-5)

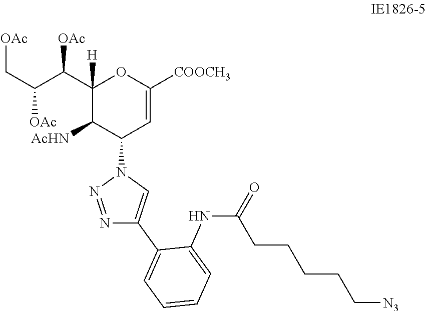 [SMILES: COC(=O)C1=C[C@H](n2cc(-c3ccccc3NC(=O)CCCCCN=[N+]=[N-])nn2)[C@@H](NC(C)=O)[C@H]([C@H](OC(C)=O)[C@@H](COC(C)=O)OC(C)=O)O1]

To a mixture of 2-aminophenyltriazole derivative IE1398-24 (400 mg, 0.70 mmol), 6-azidohexanoic acid (113 μL, 0.77 mmol) and COMU© (600 mg, 1.40 mmol) in dry DMF (10 mL) under argon, was added DIEA (370 μL, 2.1 mmol). The mixture was stirred at rt o/n and then concentrated under vacuum to give the crude product, which was purified by silica gel chromatography using ethyl acetate:hexane (3:1) as solvent to yield pure IE1826-5 (430 mg, 0.60 mmol) at 86% yield. ¹H NMR (400 MHz, MeOH-d₄): δ 1.41 (ddt, J=9.0, 6.7, 3.2 Hz, 2H), 1.54-1.63 (m, 2H), 1.67-1.77 (m, 5H), 1.89-2.06 (m, 11H), 2.41 (t, J=7.4 Hz, 2H), 3.77 (s, 3H), 4.11 (dd, J=12.5, 6.2 Hz, 1H), 4.42 (t, J=10.2 Hz, 1H), 4.57 (ddd, J=15.2, 11.6, 2.4 Hz, 2H), 5.34 (td, J=6.3, 2.7 Hz, 1H), 5.47-5.57 (m, 2H), 6.11 (d, J=2.5 Hz, 1H), 7.12 (td, J=7.6, 1.3 Hz, 1H), 7.27 (ddd, J=8.6, 7.4, 1.6 Hz, 1H), 7.62 (dd, J=8.0, 1.6 Hz, 1H), 8.17 (d, J=8.0 Hz, 1H), 8.37 (s, 1H); ¹³C NMR (101 MHz, MeOH-d₄): δ 19.25, 19.32, 19.36, 21.14, 24.84, 25.98, 28.24, 37.19, 37.24, 50.88, 51.71, 59.66, 61.77, 67.32, 70.21, 76.50, 106.75, 119.86, 121.01, 122.38, 124.24, 127.79, 128.45, 135.38, 146.10, 146.75, 170.01, 170.10, 171.00, 171.80, 172.95; LRMS [C₃₂H₄₀N₈O₁₁] (m/z): (+ve ion mode) 735.5 [M+Na]⁺

### Sodium 5-acetamido-2,6-anhydro-4-(2-(6-azidohexanamido)phenyl)-1H-1,2,3-triazol-1-yl)-3,4,5-trideoxy-D-glycero-D-galacto-non-2-enonate (IE1826-23)

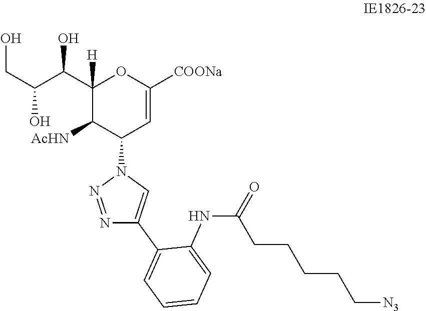

IE1826-5 (40 mg, 0.056 mmol) was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1826-23 (23 mg, 71% yield) as white powder after freeze-drying. ¹H NMR (400 MHz, D₂O): δ 1.35-1.46 (m, 2H), 1.59-1.73 (m, 4H), 1.94 (s, 3H), 2.44 (t, J=7.4 Hz, 2H), 3.34 (t, J=6.8 Hz, 2H), 3.64-3.73 (m, 2H), 3.93 (dd, J=12.0, 2.7 Hz, 1H), 4.03 (ddd, J=9.4, 6.4, 2.7 Hz, 1H), 4.45 (t, J=10.2 Hz, 1H), 4.60 (d, J=11.0 Hz, 1H), 5.61 (dd, J=9.6, 2.3 Hz, 1H), 5.88 (d, J=2.2 Hz, 1H), 7.43-7.57 (m, 3H), 7.69-7.76 (m, 1H), 8.24 (s, 1H); ¹³C NMR (101 MHz, D₂O): δ 21.73, 24.63, 25.47, 27.64, 35.90, 48.78, 50.94, 60.04, 63.09, 68.07, 69.70, 75.38, 101.61, 121.99, 125.35, 126.96, 127.53, 129.41, 129.72, 133.37, 145.15, 150.71, 168.58, 173.64, 176.15; LRMS [C₂₅H₃₁N₈NaO₈] (m/z): (+ve ion mode) 617.3 [M+Na]⁺

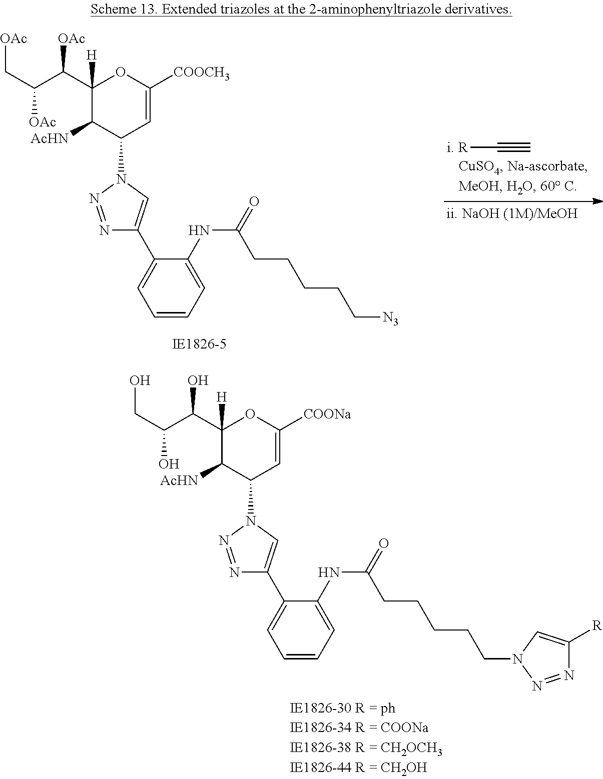

### General Procedure

The azide intermediate IE1826-5 (50 mg, 0.07 mmol) and the appropriate alkyne (0.084 mmol) were dissolved in a 4:1 mixture of MeOH:H₂O (4 mL). Copper(II)sulfate pentahydrate (3 mg, 0.014 mmol) was added to the mixture followed by sodium ascorbate (0.1 mL of freshly prepared 1 M solution in H₂O). The mixture was stirred at 60° C. for 6 h and then left to cool to rt. The mixture was then diluted with DCM (200 mL), washed with 10% NH₄OH (100 mL), followed by brine (100 mL). The organic layer was dried over anhydrous Na₂SO₄ and concentrated under vacuum to give the crude protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected triazole.

### Sodium 5-acetamido-2,6-anhydro-3,4,5-trideoxy-4-(2-(6-(4-phenyl-1H-1,2,3-triazol-1-yl) hexanamido)phenyl)-1H-1,2,3-triazol-1-yl)-D-glycero-D-galacto-non-2-enonate (IE1826-30)

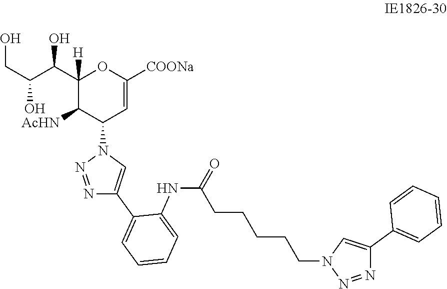

¹H NMR (400 MHz, D₂O): δ 1.25 (dt, J=14.3, 7.0 Hz, 2H), 1.67 (p, J=7.1 Hz, 2H), 1.88 (s, 3H), 1.91-1.98 (m, 2H), 2.37 (t, J=7.0 Hz, 2H), 3.63-3.73 (m, 2H), 3.91 (dd, J=12.0, 2.6 Hz, 1H), 4.02 (ddd, J=9.3, 6.2, 2.7 Hz, 1H), 4.35-4.46 (m, 3H), 4.54 (dd, J=11.2, 1.3 Hz, 1H), 5.47 (dd, J=9.7, 2.3 Hz, 1H), 5.79 (d, J=2.2 Hz, 1H), 7.27-7.33 (m, 2H), 7.39-7.53 (m, 5H), 7.59 (dd, J=7.8, 1.9 Hz, 2H), 7.97 (s, 1H), 8.15 (s, 1H); ¹³C NMR (101 MHz, D₂O): δ 21.71, 24.14, 24.49, 28.62, 35.84, 48.72, 50.15, 59.92, 63.08, 68.05, 69.69, 75.35, 101.51, 121.68, 121.90, 123.43, 125.41, 125.56, 126.81, 128.65, 129.05, 129.11, 129.31, 129.40, 133.29, 145.29, 147.23, 150.73, 168.55, 173.51, 175.54; LRMS [C₃₃H₃₇N₈NaO₈] (m/z): (+ve ion mode) 719.4 [M+Na]⁺

### Disodium 5-acetamido-2,6-anhydro-4-(2-(6-(4-carboxy-1H-1,2,3-triazol-1-yl) hexanamido)phenyl)-1H-1,2,3-triazol-1-yl)-3,4,5-trideoxy-D-glycero-D-galacto-non-2-enonate (IE1826-34)

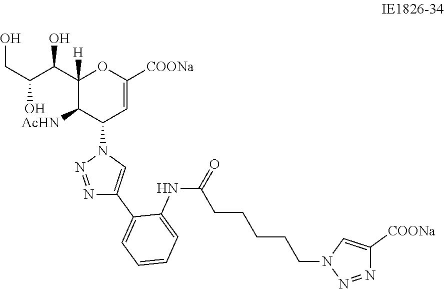

¹H NMR (400 MHz, D₂O): δ 1.32 (qd, J=8.6, 6.0 Hz, 2H), 1.64-1.74 (m, 2H), 1.91 (s, 3H), 1.97 (q, J=7.3 Hz, 2H), 2.40 (t, J=7.4 Hz, 2H), 3.65-3.76 (m, 2H), 3.92 (dd, J=12.0, 2.7 Hz, 1H), 4.03 (ddd, J=9.3, 6.3, 2.6 Hz, 1H), 4.42-4.52 (m, 3H), 4.60 (dd, J=11.0, 1.3 Hz, 1H), 5.60 (dd, J=9.7, 2.3 Hz, 1H), 5.87 (d, J=2.3 Hz, 1H), 7.42-7.55 (m, 3H), 7.71 (dd, J=7.7, 1.7 Hz, 1H), 8.22 (d, J=3.8 Hz, 2H); ¹³C NMR (101 MHz, D₂O): 6 21.71, 24.46, 24.98, 28.91, 35.72, 48.78, 50.24, 60.01, 63.08, 68.06, 69.72, 75.39, 101.69, 122.02, 125.37, 126.94, 127.08, 127.56, 129.37, 129.73, 133.28, 144.67, 145.14, 150.65, 167.67, 168.63, 173.59, 176.00; LRMS [C₂₈H₃₂N₈Na₂O₁₀] (m/z): (+ve ion mode) 709.3 [M+Na]⁺.

### Sodium 5-acetamido-2,6-anhydro-3,4,5-trideoxy-4-(2-(6-(4-(methoxymethyl)-1H-1,2,3-triazol-1-yl)hexanamido)phenyl)-1H-1,2,3-triazol-1-yl)-D-glycero-D-galacto-non-2-enonate (IE1826-38)

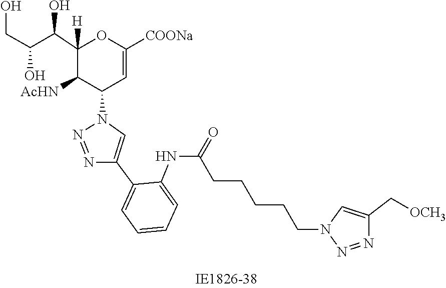

¹H NMR (400 MHz, D₂O): δ 1.27 (td, J=8.8, 4.5 Hz, 2H), 1.67 (p, J=7.3 Hz, 2H), 1.93 (d, J=9.7 Hz, 5H), 2.40 (t, J=7.1 Hz, 2H), 3.33 (s, 3H), 3.68 (dd, J=11.9, 6.3 Hz, 1H), 3.73 (d, J=9.7 Hz, 1H), 3.92 (dd, J=11.8, 2.4 Hz, 1H), 4.03 (ddd, J=9.3, 6.1, 2.4 Hz, 1H), 4.41-4.49 (m, 3H), 4.53 (s, 2H), 4.60 (d, J=10.9 Hz, 1H), 5.60 (dd, J=9.8, 2.2 Hz, 1H), 5.86 (d, J=1.8 Hz, 1H), 7.42-7.54 (m, 3H), 7.72 (d, J=7.5 Hz, 1H), 8.00 (s, 1H), 8.22 (s, 1H); ¹³C NMR (101 MHz, D₂O): δ 21.71, 24.35, 24.84, 28.83, 35.75, 48.79, 50.13, 57.26, 60.03, 63.08, 64.28, 68.06, 69.69, 75.38, 101.60, 121.95, 124.94, 124.99, 126.70, 127.43, 129.30, 129.66, 133.31, 143.58, 145.14, 150.72, 168.56, 173.59, 175.86; LRMS [C₂₉H₃₇N₈NaO₉] (m/z): (+ve ion mode) 687.3 [M+Na]⁺.

### Sodium 5-acetamido-2,6-anhydro-3,4,5-trideoxy-4-(2-(6-(4-(hydroxymethyl)-1H-1,2,3-triazol-1-yl)hexanamido)phenyl)-1H-1,2,3-triazol-1-yl)-D-glycero-D-galacto-non-2-enonate (IE1826-44)

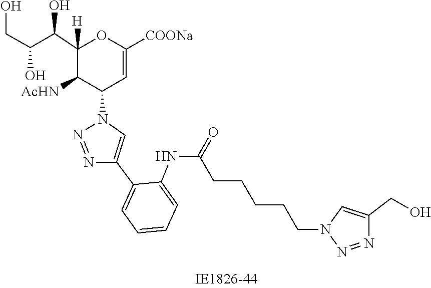

¹H NMR (400 MHz, D₂O): δ 1.22-1.34 (m, 2H), 1.67 (p, J=7.5 Hz, 2H), 1.88-2.00 (m, 5H), 2.40 (t, J=7.3 Hz, 2H), 3.65-3.76 (m, 2H), 3.93 (dd, J=11.9, 2.7 Hz, 1H), 4.03 (ddd, J=9.4, 6.5, 2.6 Hz, 1H), 4.40-4.50 (m, 3H), 4.60 (d, J=10.9 Hz, 1H), 4.66 (s, 2H), 5.60 (dd, J=9.7, 2.3 Hz, 1H), 5.86 (d, J=2.2 Hz, 1H), 7.43-7.55 (m, 3H), 7.68-7.76 (m, 1H), 7.96 (s, 1H), 8.22 (s, 1H); ¹³C NMR (101 MHz, D₂O): δ 21.72, 24.38, 24.89, 28.87, 35.75, 48.78, 50.09, 54.55, 60.02, 63.08, 68.06, 69.71, 75.39, 101.63, 122.01, 123.97, 125.12, 126.79, 127.47, 129.32, 129.69, 133.31, 145.13, 146.65, 150.69, 168.57, 173.61, 175.91; LRMS [C₂₈H₃₅N₈NaO₉] (m/z): (+ve ion mode) 673.1 [M+Na]⁺.

### Dimer Synthesis and Characterisation

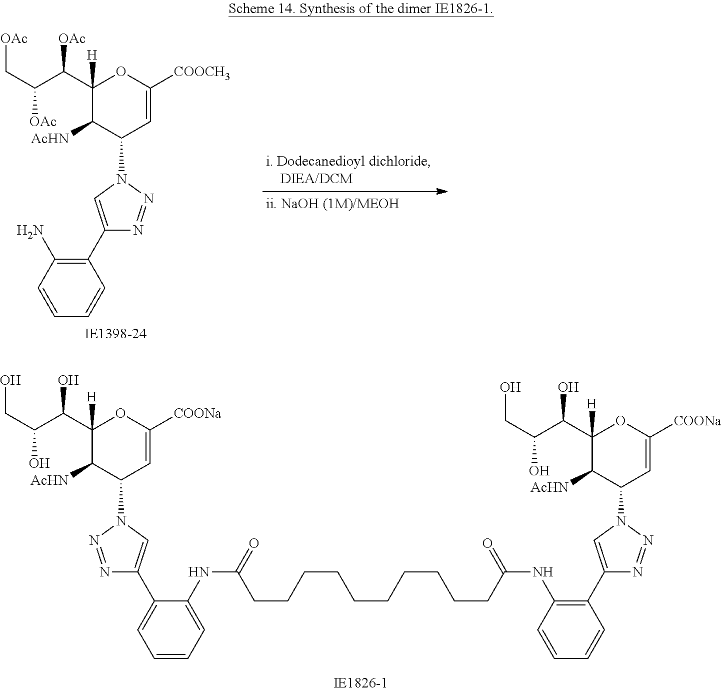

### IE1826-1

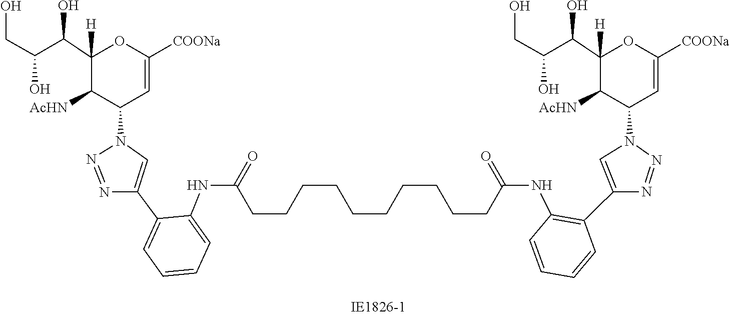

To a solution of IE1398-24 (50 mg, 0.087 mmol) in anhydrous dichloromethane (3 mL) under argon was added diisopropylethylamine (46 μL, 0.26 mmol) followed by dodecanedioyl dichloride (11 μL, 0.043 mmol). The mixture was stirred at rt for 3h, concentrated under vacuum to give the crude product, which was purified by silica gel chromatography using ethyl acetate:hexane (4:1) as solvent to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1826-1 (32 mg, 66% yield over two steps) as white powder after freeze-drying. ¹H NMR (400 MHz, D₂O): δ 1.26 (q, J=7.8, 5.9 Hz, 12H), 1.60 (q, J=6.8 Hz, 4H), 1.91 (s, 6H), 2.33-2.43 (m, 4H), 3.64-3.75 (m, 4H), 3.92 (dd, J=12.0, 2.7 Hz, 2H), 4.02 (ddd, J=9.3, 6.2, 2.7 Hz, 2H), 4.43 (dd, J=11.2, 9.3 Hz, 2H), 4.58 (dd, J=10.8, 1.4 Hz, 2H), 5.57 (dd, J=9.6, 2.4 Hz, 2H), 5.85 (d, J=2.1 Hz, 2H), 7.39 (td, J=7.5, 1.4 Hz, 2H), 7.47 (td, J=7.7, 1.6 Hz, 2H), 7.55-7.62 (m, 2H), 7.65 (dd, J=7.7, 1.6 Hz, 2H), 8.20 (s, 2H); ¹³C NMR (101 MHz, D₂O): δ 21.74, 24.99, 27.99, 28.15, 36.24, 48.78, 59.96, 63.09, 68.08, 69.69, 75.36, 101.56, 121.99, 124.56, 126.37, 127.18, 129.27, 129.62, 133.53, 145.27, 150.70, 168.52, 173.59, 176.39; LRMS [C₅₀H₆₂N₁₀Na₂O₁₆] (m/z): (+ve ion mode) 1128.0.0 [M+Na]⁺

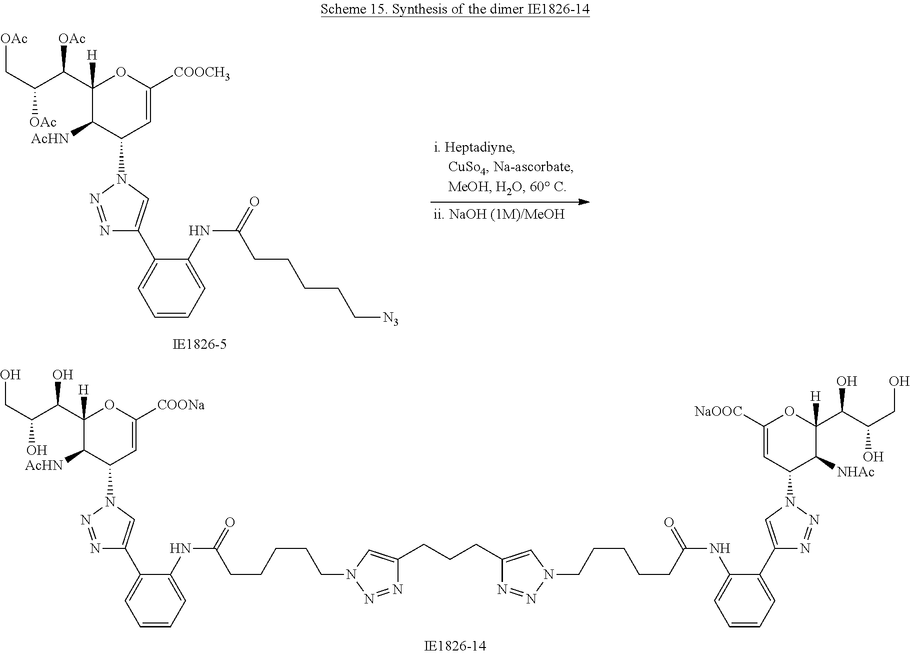

### IE1826-14

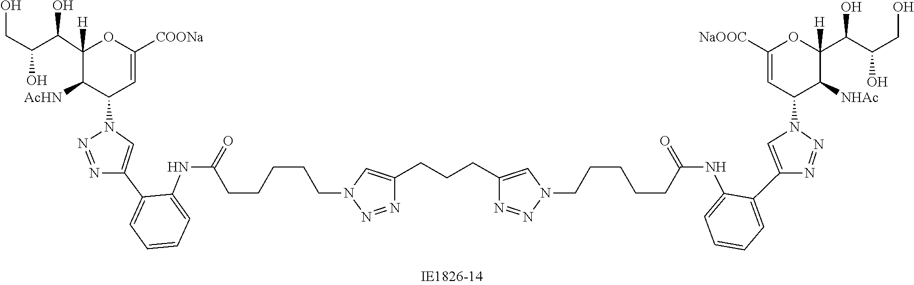

The azide derivative IE1826-5 (40 mg, 0.056 mmol) and Heptadiyne (3.0 μL, 0.028 mmol) were dissolved in a 4:1 mixture of MeOH:H₂O (4 mL). Copper(II)sulfate pentahydrate (3.0 mg, 0.22 mmol) was added to the mixture followed by sodium ascorbate (0.1 mL of freshly prepared 1 M solution in H₂O). The mixture was stirred at 60° C. for 6h and then left to cool to rt. The mixture was then concentrated under vacuum to give the crude product, which was purified by silica gel chromatography using ethyl acetate:methanol (7:1) as solvent to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1826-14 (24 mg, 69% yield over two steps) as white powder after freeze-drying. ¹H NMR (400 MHz, D₂O): δ 1.19 (ddt, J=13.6, 9.8, 6.2 Hz, 4H), 1.62 (p, J=7.4 Hz, 4H), 1.71-1.82 (m, 2H), 1.85-1.92 (m, 10H), 2.34 (q, J=6.1, 5.0 Hz, 4H), 2.51 (td, J=7.7, 3.0 Hz, 4H), 2.85 (s, 2H), 3.60-3.75 (m, 6H), 3.91 (dd, J=12.0, 2.7 Hz, 2H), 4.02 (ddd, J=9.3, 6.2, 2.6 Hz, 2H), 4.34 (t, J=6.7 Hz, 4H), 4.38-4.46 (m, 2H), 4.57 (dd, J=11.0, 1.3 Hz, 2H), 5.55 (dd, J=9.8, 2.2 Hz, 2H), 5.83 (d, J=2.2 Hz, 2H), 7.29-7.37 (m, 4H), 7.40-7.48 (m, 2H), 7.55-7.64 (m, 4H), 8.14 (d, J=9.5 Hz, 2H); ¹³C NMR (101 MHz, D₂O): 621.73, 23.77, 24.29, 24.79, 27.92, 28.80, 35.94, 48.77, 49.92, 59.95, 63.09, 68.07, 69.70, 75.38, 101.58, 121.83, 123.01, 123.82, 125.86, 126.91, 128.91, 129.41, 133.39, 145.23, 147.53, 150.73, 168.52, 173.51, 175.47; LRMS [C₅₇H₇₀N₁₆Na₂O₁₆] (m/z): (+ve ion mode) 1304.3 [M+Na]⁺

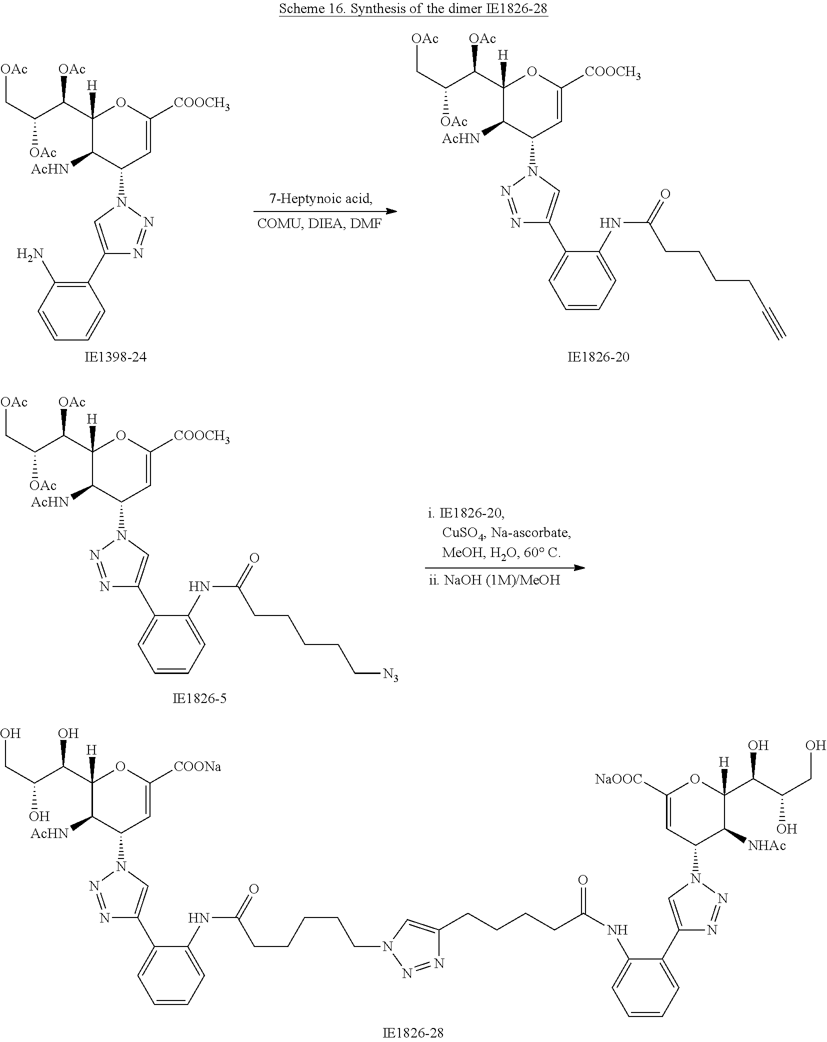

### Methyl 5-acetamido-7,8,9-tri-O-acetyl-2,6-anhydro-3,4,5-trideoxy-4-(2-(hept-6-ynamido)phenyl)-1H-1,2,3-triazol-1-yl)-D-glycero-D-galacto-non-2-enonate (IE1826-20)

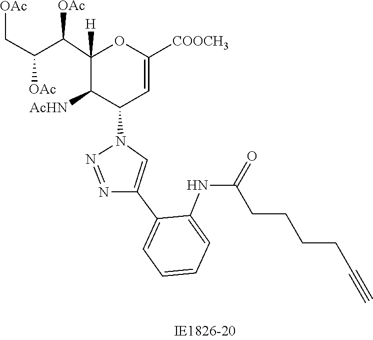 [SMILES: C#CCCCCC(=O)Nc1ccccc1-c1cn([C@H]2C=C(C(=O)OC)O[C@@H]([C@H](OC(C)=O)[C@@H](COC(C)=O)OC(C)=O)[C@@H]2NC(C)=O)nn1]

To a mixture of 2-aminophenyltriazole derivative IE1398-24 (200 mg, 0.35 mmol), 7-heptynoic acid (55 μL, 0.42 mmol) and COMU© (300 mg, 0.7 mmol) in dry DMF (5 mL) under argon, was added DIEA (240 μL, 1.4 mmol). The mixture was stirred at rt o/n and then concentrated under vacuum to give the crude product, which was purified by silica gel chromatography using ethyl acetate:hexane (2:1) as solvent to yield pure IE1826-20 (190 mg, 0.28 mmol) at 80% yield. LRMS [C₃₃H₃₉N₅O₁₁] (m/z): (+ve ion mode) 735.5 [M+Na]⁺

### IE1826-28

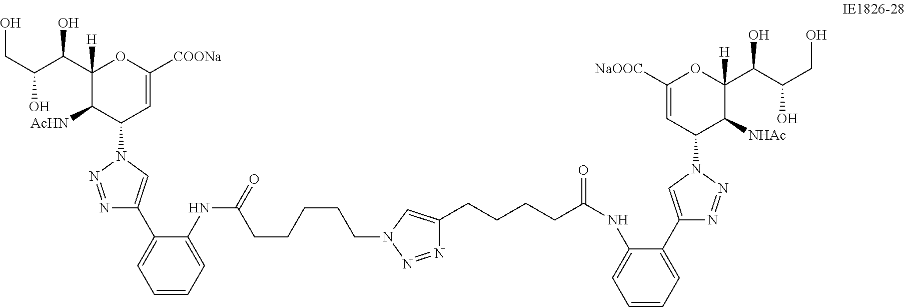

The azide derivative IE1826-5 (32 mg, 0.044 mmol) and the alkyne derivative IE1826-20 (30 mg, 0.044 mmol) were dissolved in a 4:1 mixture of MeOH:H₂O (4 mL). Copper(II)sulfate pentahydrate (2.5 mg, 0.01 mmol) was added to the mixture followed by sodium ascorbate (0.1 mL of freshly prepared 1 M solution in H₂O). The mixture was stirred at 60° C. for 6h and then left to cool to rt. The mixture was then concentrated under vacuum to give the crude product, which was purified by silica gel chromatography using ethyl acetate:methanol (10:1) as solvent to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 2% acetonitrile/water as solvent to yield the pure deprotected product IE1826-28 (17 mg, 65% yield over two steps) as white powder after freeze-drying. ¹H NMR (400 MHz, D₂O): δ 1.18-1.25 (m, 2H), 1.60 (q, J=7.7, 7.2 Hz, 6H), 1.85-1.96 (m, 8H), 2.34 (dt, J=14.6, 7.6 Hz, 4H), 2.64-2.74 (m, 2H), 3.60-3.72 (m, 4H), 3.92 (dd, J=12.0, 2.7 Hz, 2H), 4.02 (ddd, J=9.3, 6.2, 2.6 Hz, 2H), 4.35-4.48 (m, 4H), 4.53-4.64 (m, 2H), 5.49-5.59 (m, 2H), 5.82 (dd, J=12.3, 2.3 Hz, 2H), 7.33-7.55 (m, 6H), 7.57-7.63 (m, 1H), 7.65 (dd, J=7.7, 1.4 Hz, 1H), 7.80 (s, 1H), 8.10 (s, 1H), 8.15 (s, 1H); LRMS [C₅₁H₆₁N₁₃Na₂O₁₆] (m/z): (+ve ion mode) 1181.0 [M+Na]⁺

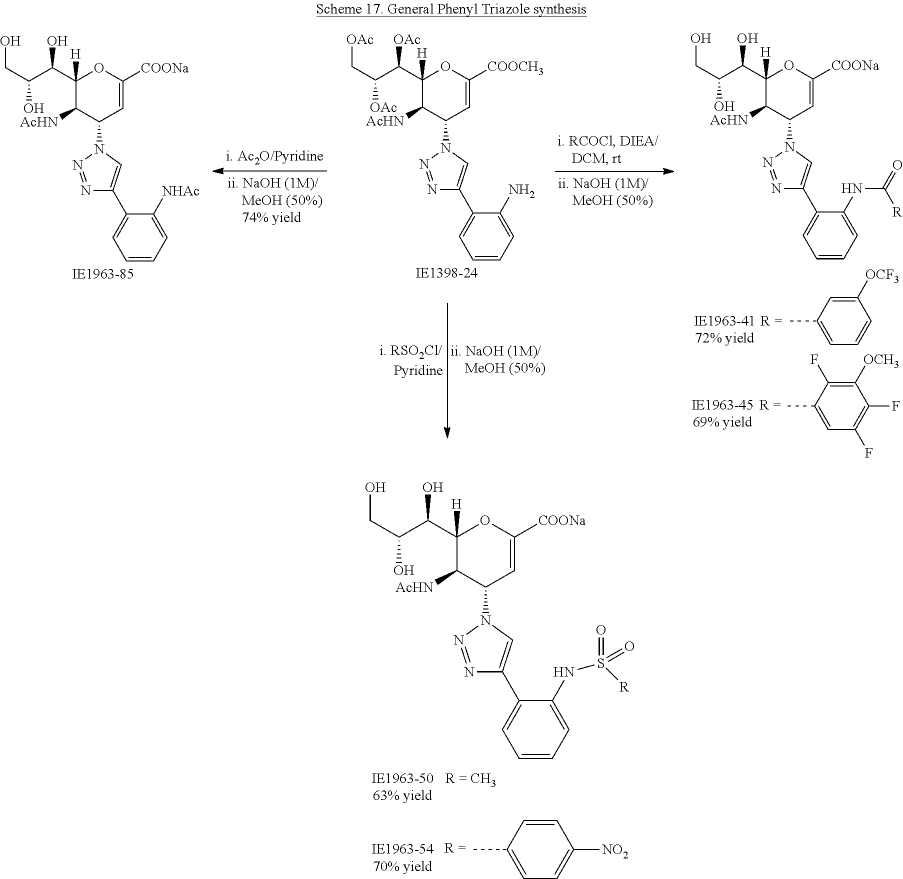

### IE1963-85

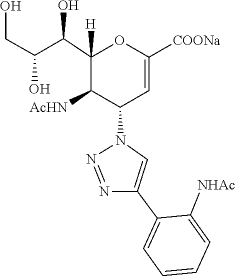

To a solution of the amine IE1398-24 (60 mg, 0.105 mmol) in pyridine (2 mL) was added Ac₂O (50 μL, 0.52 mmol), and the reaction mixture was stirred at rt under argon o/n. The reaction mixture was concentrated under vacuum, and the crude product was purified by silica gel chromatography to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 10% methanol/water as solvent to yield the pure deprotected product IE1963-85 (74% yield over two steps) after freeze-drying. ¹H NMR (400 MHz, D₂O): δ 1.93 (s, 4H), 2.15 (s, 3H), 3.64-3.76 (m, 2H), 3.92 (dd, J=11.9, 2.7 Hz, 1H), 4.03 (ddd, J=9.3, 6.2, 2.6 Hz, 1H), 4.46 (t, J=10.3 Hz, 1H), 4.60 (d, J=10.9 Hz, 1H), 5.60 (dd, J=9.6, 2.3 Hz, 1H), 5.88 (d, J=2.2 Hz, 1H), 7.44-7.54 (m, 3H), 7.73 (dd, J=7.3, 1.5 Hz, 1H), 8.25 (s, 1H); ¹³C NMR (101 MHz, D₂O): δ 21.70, 22.32, 48.79, 60.01, 63.07, 68.06, 69.72, 75.37, 101.81, 122.07, 125.44, 127.09, 127.61, 129.26, 129.69, 133.28, 145.00, 150.57, 168.70, 173.57, 173.63; LRMS [C₂₁H₂₄N₅NaO₈] (m/z): (+ve ion mode) 519.3 [M+Na]⁺.

### Synthesis of the Amides IE1963-41 and IE1963-45

To a solution of the amine IE1398-24 (60 mg, 0.105 mmol) in anhydrous DCM (2 mL) was added DIEA (90 μL, 0.52 mmol), followed by portionwise addition of the acid chloride (2 eq) and the reaction mixture was stirred at rt under argon o/n. The reaction mixture was concentrated under vacuum, and the crude product was purified by silica gel chromatography to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 10% methanol/water as solvent to yield the pure deprotected product.

### IE1963-41

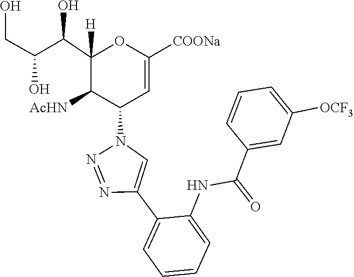

¹H NMR (400 MHz, D₂O): δ 1.79 (s, 3H), 3.65 (dq, J=11.4, 5.9 Hz, 2H), 3.85-3.92 (m, 1H), 3.98 (ddd, J=9.4, 6.1, 2.7 Hz, 1H), 4.36 (t, J=10.1 Hz, 1H), 4.54 (d, J=10.9 Hz, 1H), 5.54 (dd, J=9.3, 2.6 Hz, 1H), 5.77 (d, J=2.2 Hz, 1H), 7.34 (t, J=7.7 Hz, 1H), 7.43 (t, J=7.6 Hz, 1H), 7.51 (d, J=7.1 Hz, 1H), 7.58 (t, J=7.8 Hz, 1H), 7.64 (d, J=7.7 Hz, 1H), 7.72 (s, 1H), 7.82 (dd, J=17.3, 7.8 Hz, 2H), 8.27 (s, 1H); ¹³C NMR (101 MHz, D₂O): 6 21.53, 48.69, 59.82, 63.06, 68.03, 69.66, 75.33, 99.99, 101.39, 118.92, 119.92, 121.45, 122.91, 124.83, 125.63, 126.73, 128.69, 129.44, 130.73, 133.70, 135.42, 145.94, 149.00, 150.69, 165.53, 168.48, 173.47; LRMS [C₂₇H₂₅F₃N₅NaO₉] (m/z): (+ve ion mode) 665.6 [M+Na]⁺.

### IE1963-45

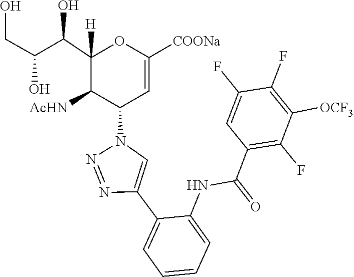

¹H NMR (400 MHz, D₂O): δ 1.87 (s, 3H), 3.64-3.73 (m, 2H), 3.92 (dd, J=12.0, 2.7 Hz, 1H), 4.02 (ddd, J=9.4, 6.3, 2.7 Hz, 1H), 4.08 (s, 3H), 4.40 (t, J=10.3 Hz, 1H), 4.53-4.61 (m, 1H), 5.59 (dd, J=9.9, 2.3 Hz, 1H), 5.81 (d, J=2.2 Hz, 1H), 7.35-7.42 (m, 1H), 7.46 (dd, J=7.6, 1.5 Hz, 1H), 7.49-7.54 (m, 1H), 7.72 (td, J=8.1, 1.5 Hz, 2H), 8.29 (s, 1H); ¹³C NMR (101 MHz, D₂O): δ 21.60, 48.70, 59.93, 62.46, 63.08, 68.05, 69.68, 75.39, 101.51, 110.56 (d, J=21.4 Hz), 121.79, 124.55, 126.13, 127.56, 129.18, 129.68, 133.21, 145.26, 148.43, 150.64, 151.55, 163.74, 168.53, 173.60, 174.00; LRMS [C₂₇H₂₅F₃N₅NaO₉] (m/z): (+ve ion mode) 665.6 [M+Na]⁺.

### Synthesis of the Sulfonamides IE1963-50 and IE1963-54

To a solution of the amine IE1398-24 (60 mg, 0.105 mmol) in dry pyridine (2 mL) was added the sulfonyl chloride (1.2 eq) and the reaction mixture was stirred at rt under argon o/n. The reaction mixture was concentrated under vacuum, and the crude product was purified by silica gel chromatography to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 10% methanol/water as solvent to yield the pure deprotected product.

### IE1963-50

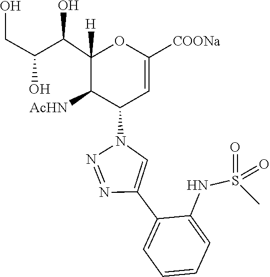

¹H NMR (400 MHz, D₂O): 61.93 (s, 3H), 2.86 (s, 3H), 3.64-3.74 (m, 2H), 3.93 (dd, J=11.9, 2.7 Hz, 1H), 4.03 (ddd, J=9.3, 6.3, 2.6 Hz, 1H), 4.50 (t, J=10.2 Hz, 1H), 4.60 (d, J=10.9 Hz, 1H), 5.59 (dd, J=9.7, 2.4 Hz, 1H), 5.94 (d, J=2.2 Hz, 1H), 7.16 (ddd, J=8.3, 5.0, 3.6 Hz, 1H), 7.34-7.40 (m, 2H), 7.93 (dd, J=7.3, 0.8 Hz, 1H), 8.65 (s, 1H); ¹³C NMR (101 MHz, D₂O): 621.77, 39.67, 48.72, 59.66, 63.11, 68.15, 69.76, 75.42, 102.34, 121.93, 123.89, 123.92, 124.06, 127.91, 129.33, 142.91, 145.41, 150.17, 168.85, 173.75; LRMS [C₂₀H₂₄N₅NaO₉S] (m/z): (+ve ion mode) 555.5 [M+Na]⁺.

### IE1963-54

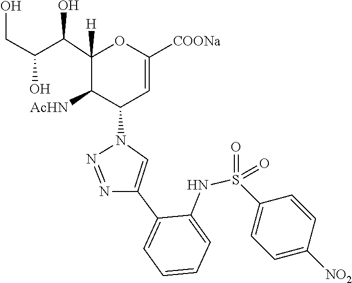

¹H NMR (400 MHz, D₂O): δ 1.89 (s, 3H), 3.64-3.73 (m, 2H), 3.93 (dd, J=12.0, 2.7 Hz, 1H), 4.03 (ddd, J=9.3, 6.3, 2.7 Hz, 1H), 4.36 (t, J=10.3 Hz, 1H), 4.53-4.61 (m, 1H), 5.50 (dd, J=9.9, 2.3 Hz, 1H), 5.87 (d, J=2.2 Hz, 1H), 7.15 (td, J=7.4, 1.4 Hz, 1H), 7.23 (d, J=7.8 Hz, 1H), 7.28-7.34 (m, 1H), 7.66 (d, J=8.8 Hz, 2H), 7.76 (dd, J=7.8, 1.7 Hz, 1H), 8.17 (d, J=8.8 Hz, 2H), 8.32 (s, 1H); ¹³C NMR (101 MHz, D₂O): δ 21.70, 48.64, 59.55, 63.11, 68.12, 69.74, 75.40, 101.92, 122.69, 123.42, 123.99, 125.04, 125.88, 126.43, 127.97, 129.28, 142.67, 145.17, 148.39, 149.76, 150.41, 168.75, 173.65; LRMS [C₂₅H₂₅N₆NaO₁₁S] (m/z): (+ve ion mode) 662.6 [M+Na]⁺.

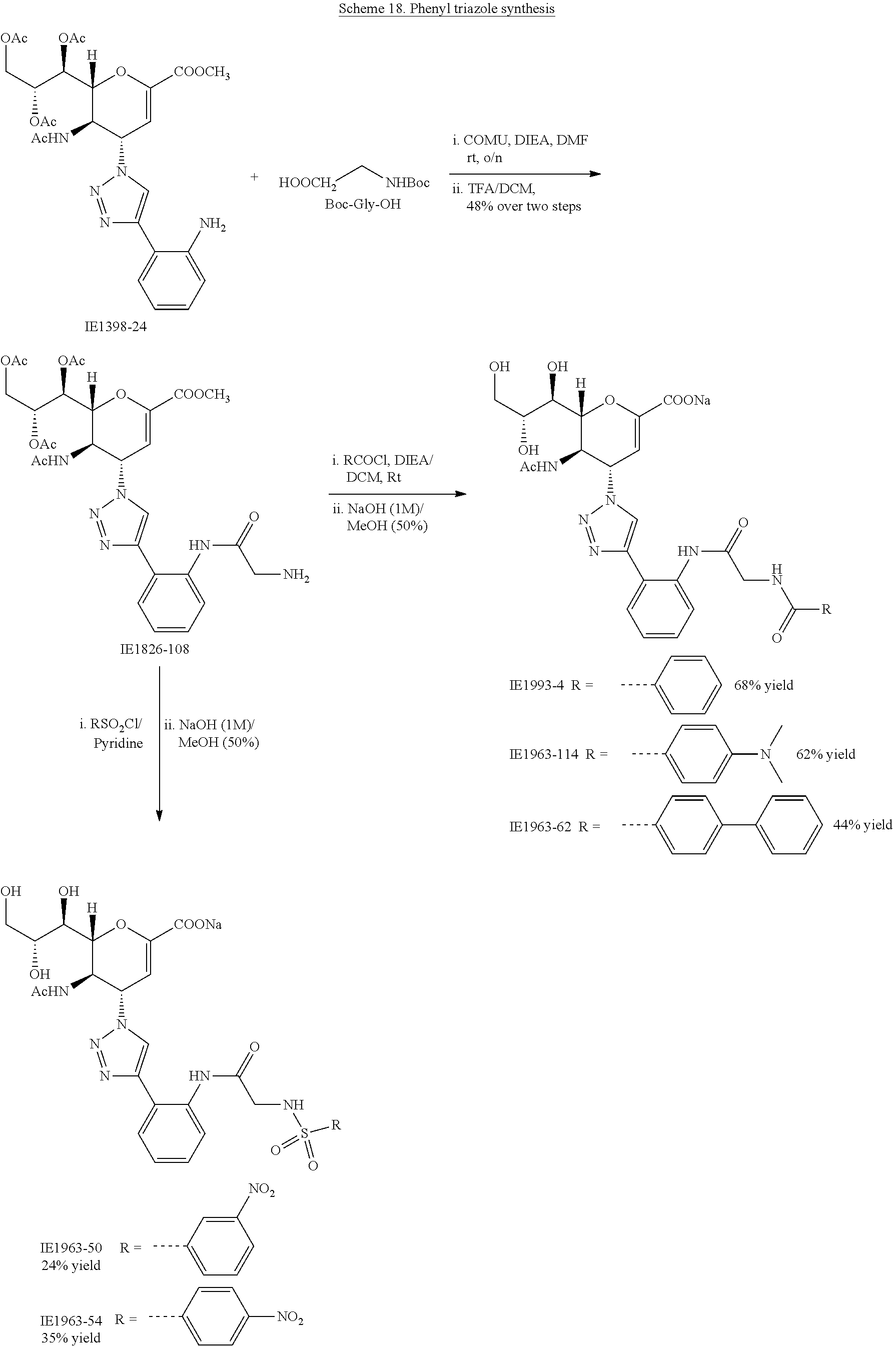

### IE1826-108

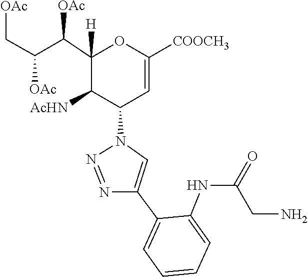 [SMILES: COC(=O)C1=C[C@H](n2cc(-c3ccccc3NC(=O)CN)nn2)[C@@H](NC(C)=O)[C@H]([C@H](OC(C)=O)[C@@H](COC(C)=O)OC(C)=O)O1]

To a solution of the amine IE1398-24 (1.0 g, 1.744 mmol) in dry DMF (6 ml) was added Boc-Gly-OH (0.61 g, 3.48 mmol, 2.0 eq) followed by DIEA (1.21 ml, 6.976 mmol, 4.0 eq) and COMU (1.49 g, 3.48 mmol, 2.0 eq), and the reaction mixture was stirred at rt o/n. The mixture was concentrated under vacuum, and the crude product was purified by silica gel chromatography (Hexane/acetone 3:2) to yield the pure N-Boc protected product. To a solution of the Boc-protected product (1.0 g, 1.369 mmol) in anhydrous DCM, was added TFA (2.1 ml, 27.37 mmol, 20 eq) at 0° C. and the reaction mixture was allowed to warm to rt and stirred under argon o/n. The reaction was diluted with acetonitrile and then after cooling down to 0° C. quenched by adding powdered sodium carbonate and stirred for 5 mins and filtered, washed with water and the organic solvent was dried over anhydrous Na₂SO₄ and concentrated under reduced pressure. The crude amine was purified by flash chromatography (Ethyl acetate/methanol/water; 7/1/0.5) to yield the pure amine IE1826-108 in 48% yield over two steps. ¹H NMR (400 MHz, CD₃OD): δ 1.80 (s, 3H), 2.04 (s, 3H), 2.05 (s, 3H), 2.07 (s, 3H), 3.83 (s, 3H), 3.93 (s, 2H), 4.18 (dd, J=12.5, 6.2 Hz, 1H), 4.53 (t, J=10.1 Hz, 1H), 4.63 (ddd, J=12.5, 9.7, 2.3 Hz, 2H), 5.41 (td, J=6.4, 2.7 Hz, 1H), 5.56 (td, J=8.2, 7.4, 2.3 Hz, 2H), 6.18 (d, J=2.2 Hz, 1H), 7.22-7.32 (m, 1H), 7.34-7.44 (m, 1H), 7.67 (dd, J=7.8, 1.6 Hz, 1H), 8.07 (d, J=8.1 Hz, 1H), 8.42 (s, 1H); ¹³C NMR (101 MHz, CD₃OD): δ 19.24, 19.33, 19.34, 21.14, 41.03, 51.71, 59.82, 61.75, 67.32, 70.12, 76.45, 106.81, 121.61, 121.71, 123.35, 125.31, 128.43, 128.71, 134.45, 145.98, 146.12, 161.53, 164.50, 170.01, 170.09, 171.02, 172.04; LRMS [C₂₈H₃₄N₆O₁₁] (m/z): (+ve ion mode) 630.8 [M+H]⁺.

### Synthesis of the Amides IE1993-4, IE1963-114 and IE1963-62

To a solution of the amine IE1826-108 (50 mg, 0.08 mmol) in anhydrous DCM (2 mL) was added DIEA (90 μL, 0.52 mmol), followed by portionwise addition of the acid chloride (2 eq) and the reaction mixture was stirred at rt under argon o/n. The reaction mixture was concentrated under vacuum, and the crude product was purified by silica gel chromatography to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 10% methanol/water as solvent to yield the pure deprotected product.

### IE1993-4

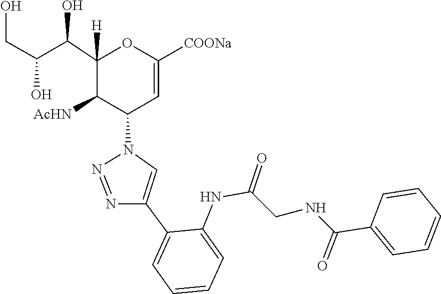

¹H NMR (400 MHz, D₂O): δ 1.86 (s, 3H), 3.64-3.74 (m, 2H), 3.92 (dd, J=12.0, 2.7 Hz, 1H), 4.03 (ddd, J=9.3, 6.2, 2.6 Hz, 1H), 4.16-4.29 (m, 2H), 4.38-4.48 (m, 1H), 4.55 (d, J=11.0 Hz, 1H), 5.41 (dd, J=9.8, 2.4 Hz, 1H), 5.75 (d, J=2.2 Hz, 1H), 7.37 (t, J=7.5 Hz, 1H), 7.47 (td, J=7.7, 1.6 Hz, 1H), 7.56 (t, J=7.6 Hz, 2H), 7.62-7.67 (m, 2H), 7.83 (d, J=8.2 Hz, 1H), 7.87 (dd, J=7.4, 1.8 Hz, 2H), 8.29 (s, 1H); ¹³C NMR (101 MHz, D₂O): δ 21.68, 43.99, 48.66, 59.89, 63.08, 68.09, 69.70, 75.33, 101.90, 122.41, 123.00, 124.91, 126.78, 127.43, 128.80, 128.88, 129.46, 132.39, 132.65, 133.18, 145.23, 150.35, 168.68, 170.94, 171.11, 173.59; LRMS [C₂₈H₂₉N₆NaO₉] (m/z): (+ve ion mode) 638.4 [M+Na]⁺.

### IE1963-114

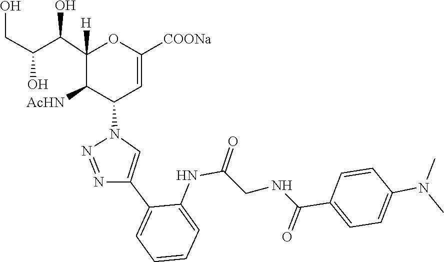

¹H NMR (400 MHz, D₂O): 61.84 (s, 3H), 2.99 (s, 6H), 3.68 (ddd, J=14.3, 9.3, 3.8 Hz, 2H), 3.91 (dd, J=12.0, 2.7 Hz, 1H), 4.01 (ddd, J=9.3, 6.3, 2.7 Hz, 1H), 4.15 (q, J=16.8 Hz, 2H), 4.36 (t, J=10.3 Hz, 1H), 4.51 (dd, J=11.1, 1.3 Hz, 1H), 5.05 (dd, J=9.8, 2.3 Hz, 1H), 5.56 (d, J=2.2 Hz, 1H), 6.87 (d, J=9.0 Hz, 2H), 7.38 (td, J=7.6, 1.4 Hz, 1H), 7.49 (td, J=7.7, 1.7 Hz, 1H), 7.62-7.67 (m, 1H), 7.77 (d, J=8.9 Hz, 2H), 7.85 (d, J=8.1 Hz, 1H), 8.13 (s, 1H); ¹³C NMR (101 MHz, D₂O): δ 21.62, 39.60, 44.21, 48.72, 59.88, 63.13, 68.06, 69.71, 75.43, 101.99, 112.09, 118.79, 122.54, 123.02, 124.76, 126.72, 129.12, 129.18, 129.56, 133.08, 144.69, 150.13, 153.81, 168.39, 170.51, 171.37, 173.56; LRMS [C₃₀H₃₄N₇NaO₉] (m/z): (+ve ion mode) 681.5 [M+Na]⁺.

### IE1963-62

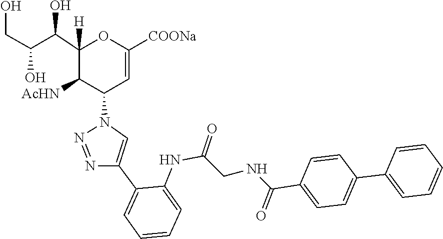

¹H NMR (400 MHz, D₂O): 61.80 (s, 3H), 3.29 (d, J=1.7 Hz, 2H), 3.84 (t, J=11.9 Hz, 1H), 3.95 (s, 1H), 4.13-4.26 (m, 2H), 4.35 (t, J=10.1 Hz, 1H), 4.44 (d, J=11.0 Hz, 1H), 5.22 (d, J=9.4 Hz, 1H), 5.62 (s, 1H), 7.36 (d, J=7.8 Hz, 1H), 7.42-7.56 (m, 4H), 7.61 (d, J=7.7 Hz, 1H), 7.71 (d, J=7.4 Hz, 2H), 7.78 (d, J=7.5 Hz, 2H), 7.89 (dd, J=14.9, 8.5 Hz, 3H), 8.24 (s, 1H); LRMS [C₃₄H₃₃N₆NaO₉] (m/z): (+ve ion mode) 714.6 [M+Na]⁺.

### Synthesis of the Sulfonamides IE1993-9 and IE1963-99

To a solution of the amine IE1826-108 (50 mg, 0.08 mmol) in anhydrous DCM (2 mL) was added DMAP (cat.) followed by the sulfonyl chloride (1.2 eq) and the reaction mixture was stirred at rt under argon o/n. The reaction mixture was concentrated under vacuum, and the crude product was purified by silica gel chromatography to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 10% methanol/water as solvent to yield the pure deprotected product.

### IE1993-9

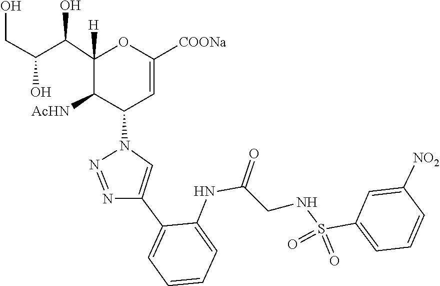

¹H NMR (400 MHz, D₂O): 61.92 (s, 3H), 3.64-3.76 (m, 2H), 3.86 (s, 2H), 3.93 (dd, J=11.9, 2.7 Hz, 1H), 4.03 (ddd, J=9.3, 6.3, 2.8 Hz, 1H), 4.50 (t, J=10.2 Hz, 1H), 4.60 (d, J=10.9 Hz, 1H), 5.63 (dd, J=9.7, 2.2 Hz, 1H), 5.91 (d, J=2.2 Hz, 1H), 7.34-7.46 (m, 2H), 7.61-7.76 (m, 3H), 8.16 (d, J=7.8 Hz, 1H), 8.34 (dt, J=8.2, 1.6 Hz, 1H), 8.37 (s, 1H), 8.57 (t, J=2.0 Hz, 1H); ¹³C NMR (101 MHz, D₂O): δ 21.75, 46.94, 48.67, 59.95, 63.10, 68.12, 69.71, 75.44, 101.88, 121.67, 122.17, 122.81, 124.53, 126.77, 127.05, 128.82, 129.43, 130.84, 132.58, 133.01, 141.90, 145.19, 147.80, 150.50, 168.71, 171.36, 173.71; LRMS [C₂₇H₂₈N₇NaO₁₂S] (m/z): (+ve ion mode) 719.5 [M+Na]⁺.

### IE1963-99

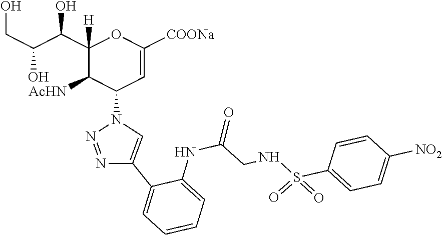

¹H NMR (400 MHz, D₂O): 61.93 (s, 3H), 3.65-3.75 (m, 2H), 3.85 (s, 2H), 3.93 (dd, J=11.9, 2.7 Hz, 1H), 4.03 (ddd, J=9.3, 6.3, 2.6 Hz, 1H), 4.52 (dd, J=10.9, 9.5 Hz, 1H), 4.61 (dd, J=11.0, 1.0 Hz, 1H), 5.64 (dd, J=9.6, 2.3 Hz, 1H), 5.92 (d, J=2.2 Hz, 1H), 7.37 (td, J=7.6, 1.4 Hz, 1H), 7.44 (td, J=7.8, 1.7 Hz, 1H), 7.64 (dd, J=8.1, 1.3 Hz, 1H), 7.70 (dd, J=7.7, 1.6 Hz, 1H), 7.99 (d, J=8.8 Hz, 2H), 8.28 (d, J=8.8 Hz, 2H), 8.39 (s, 1H); ¹³C NMR (101 MHz, D₂O): δ 21.75, 46.82, 48.65, 60.03, 63.09, 68.12, 69.71, 75.46, 101.88, 122.17, 123.10, 124.52, 124.75, 126.90, 127.82, 128.85, 129.46, 132.97, 145.17, 145.79, 149.50, 150.53, 168.71, 171.26, 173.68; LRMS [C₂₇H₂₈N₇NaO₁₂S] (m/z): (+ve ion mode) 719.5 [M+Na]⁺.

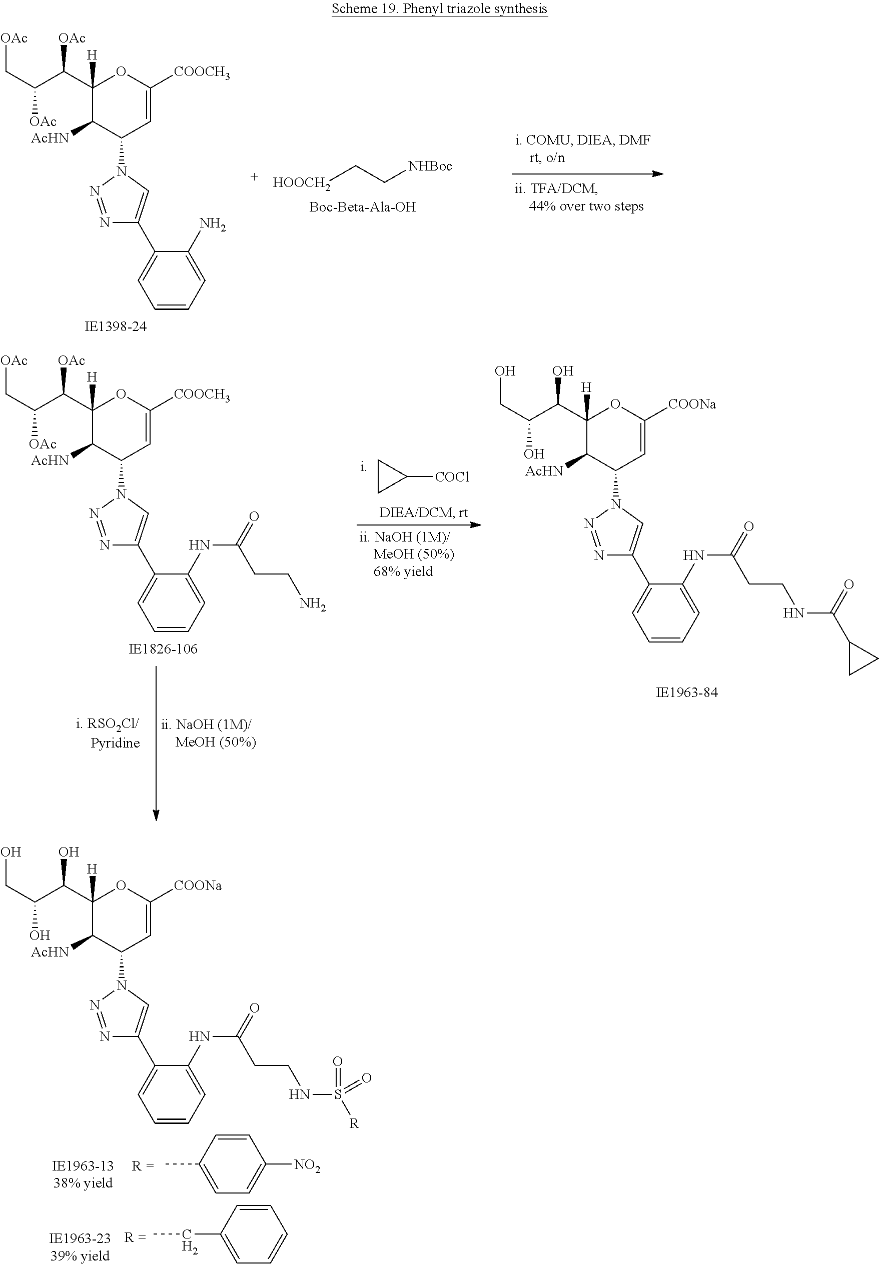

### IE1826-106

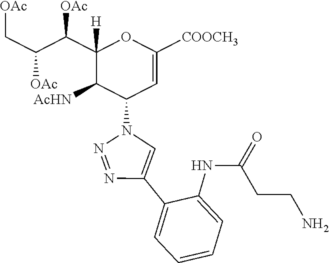 [SMILES: COC(=O)C1=C[C@H](n2cc(-c3ccccc3NC(=O)CCN)nn2)[C@@H](NC(C)=O)[C@H]([C@H](OC(C)=O)[C@@H](COC(C)=O)OC(C)=O)O1]

Boc-β-Alanine-OH (333 mg, 1.76 mmol) was activated with DIEA (0.61 mL, 3.5 mmol) and COMU (750 mg, 1.76 mmol) in DMF (3 ml), and then added to a stirred solution of the amine IE1398-24 (504 mg, 0.879 mmol) in DMF (3 ml). The mixture was stirred at rt o/n, then concentrated under vacuum. The crude product was then dissolved in water, extracted with ethyl acetate (50 mL×4), the organic layers were combined, dried with over magnesium sulfate, concentrated under vacuum and then purified by silica gel chromatography using ethyl acetate/hexane (4:1). To a solution of the Boc-protected product in anhydrous DCM (15 mL), was added TFA (2.0 ml, 26.0 mmol, 20 eq) at 0° C. and the reaction mixture was allowed to warm to rt and stirred under argon o/n. The reaction was diluted with acetonitrile and then after cooling down to 0° C. quenched by adding powdered sodium carbonate and stirred for 5 mins and filtered, washed with water and the organic solvent was dried over anhydrous Na₂SO₄ and concentrated under reduced pressure. The crude amine was purified by flash chromatography (acetone/methanol (4:1) to yield the pure amine IE1826-106 in 44% yield over two steps.

¹H NMR (400 MHz, CD₃OD): δ 1.80 (s, 3H), 2.04 (s, 3H), 2.05 (s, 3H), 2.07 (s, 3H), 2.64 (t, J=6.9 Hz, 2H), 3.05 (t, J=7.3 Hz, 2H), 3.83 (s, 3H), 4.18 (dd, J=12.5, 6.3 Hz, 1H), 4.52 (t, J=10.1 Hz, 1H), 4.61 (dd, J=12.5, 2.7 Hz, 1H), 4.67 (dd, J=10.8, 2.0 Hz, 1H), 5.41 (td, J=6.4, 2.7 Hz, 1H), 5.53-5.66 (m, 2H), 6.18 (d, J=2.2 Hz, 1H), 7.21 (t, J=7.6 Hz, 1H), 7.30-7.40 (m, 1H), 7.63-7.73 (m, 1H), 8.21 (d, J=8.1 Hz, 1H), 8.50 (s, 1H); ¹³C NMR (101 MHz, CD₃OD): δ 19.25, 19.33, 19.38, 21.18, 33.72, 38.98, 51.72, 59.70, 61.78, 67.33, 70.13, 76.46, 106.88, 120.02, 121.68, 122.56, 124.47, 127.89, 128.48, 135.17, 145.98, 146.59, 161.56, 170.00, 170.10, 171.01, 171.39, 171.83; LRMS [C₂₉H₃₆N₆O₁₁] (m/z): (+ve ion mode) 645.3 [M+H]⁺.

### Synthesis of the Amide IE1963-84

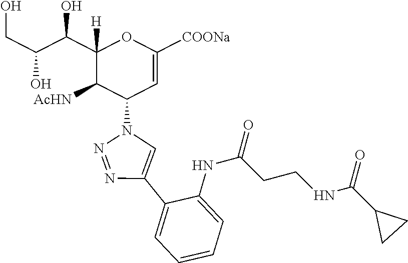

To a solution of the amine IE1826-106 (60 mg, 0.093 mmol) in anhydrous DCM (2 mL) was added DIEA (80 μL, 0.47 mmol), followed by dropwise addition of cyclopropane carbonyl chloride (2 eq) and the reaction mixture was stirred at rt under argon o/n. The reaction mixture was concentrated under vacuum, and the crude product was purified by silica gel chromatography to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 10% methanol/water as solvent to yield the pure deprotected product. ¹H NMR (400 MHz, D₂O): δ 0.76-0.85 (m, 4H), 1.59 (dq, J=7.2, 6.0, 5.5 Hz, 1H), 1.93 (s, 3H), 2.57-2.67 (m, 2H), 3.53 (t, J=6.5 Hz, 2H), 3.65-3.75 (m, 2H), 3.92 (dd, J=12.0, 2.7 Hz, 1H), 4.03 (ddd, J=9.2, 6.2, 2.6 Hz, 1H), 4.45 (dd, J=10.9, 9.6 Hz, 1H), 4.55-4.63 (m, 1H), 5.60 (dd, J=9.6, 2.3 Hz, 1H), 5.88 (d, J=2.2 Hz, 1H), 7.45 (td, J=7.6, 1.5 Hz, 1H), 7.51 (td, J=7.6, 1.7 Hz, 1H), 7.59 (dd, J=8.0, 1.4 Hz, 1H), 7.71 (dd, J=7.6, 1.6 Hz, 1H), 8.31 (s, 1H); ¹³C NMR (101 MHz, D₂O): δ 6.64, 14.03, 21.72, 35.83, 36.24, 48.78, 60.03, 63.07, 68.06, 69.70, 75.36, 101.74, 122.13, 124.73, 126.52, 127.39, 129.32, 129.65, 133.25, 145.33, 150.62, 168.66, 173.31, 173.60, 177.22; LRMS [C₂₆H₃₁N₆NaO₉] (m/z): (+ve ion mode) 616.5 [M+Na]⁺.

### Synthesis of the Sulfonamides IE1993-13 and IE1993-23

To a solution of the amine IE1826-106 (60 mg, 0.093 mmol) in anhydrous DCM (2 mL) was added DMAP (cat.) followed by the sulfonyl chloride (1.2 eq) and the reaction mixture was stirred at rt under argon o/n. The reaction mixture was concentrated under vacuum, and the crude product was purified by silica gel chromatography to yield pure protected product. The protected product was suspended in a 1:1 mixture of MeOH:H₂O (2 mL). To this suspension at 0° C. was added dropwise a NaOH solution (1.0 M) until pH ˜14. The temperature was gradually raised to rt and the mixture was stirred at rt overnight. The solution was then acidified with Amberlite® IR-120 (H⁺) resin (to pH=5), filtered and washed with MeOH (10 mL) and H₂O (10 mL). The combined filtrate and washings were then concentrated under vacuum, then diluted with distilled water (5 mL) and adjusted to pH=8.0 using 0.05 M NaOH to convert the compound to its sodium salt. Finally, the compound was purified on a C18-GracePure™ cartridge using 10% methanol/water as solvent to yield the pure deprotected product.

### IE1993-13

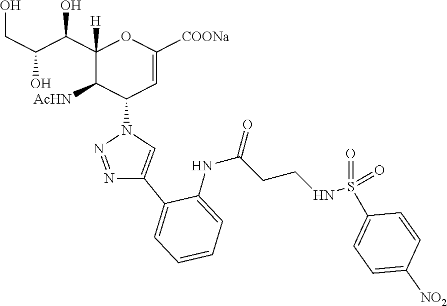

¹H NMR (400 MHz, D₂O): δ 1.95 (s, 3H), 2.52-2.61 (m, 2H), 3.43 (t, J=6.1 Hz, 2H), 3.65-3.78 (m, 2H), 3.93 (dd, J=12.0, 2.7 Hz, 1H), 4.04 (ddd, J=9.3, 6.3, 2.6 Hz, 1H), 4.49 (t, J=10.2 Hz, 1H), 4.60 (d, J=10.9 Hz, 1H), 5.60 (dd, J=9.7, 2.3 Hz, 1H), 5.89 (d, J=2.2 Hz, 1H), 7.27 (t, J=7.6 Hz, 1H), 7.37 (td, J=7.7, 1.6 Hz, 1H), 7.58 (dd, J=8.3, 1.3 Hz, 1H), 7.66 (d, J=8.1 Hz, 1H), 7.89 (d, J=8.8 Hz, 2H), 8.08 (d, J=8.8 Hz, 2H), 8.33 (s, 1H); ¹³C NMR (101 MHz, D₂O): δ 21.78, 37.38, 39.48, 48.68, 60.00, 63.10, 68.10, 69.73, 75.48, 101.81, 121.55, 121.98, 124.06, 124.42, 126.30, 127.69, 128.47, 129.29, 133.45, 145.54, 145.58, 149.22, 150.62, 168.68, 172.10, 173.60; LRMS [C₂₁H₂₃N₃Na₂O₉] (m/z): (+ve ion mode) 733.5 [M+Na]⁺.

### IE1993-23

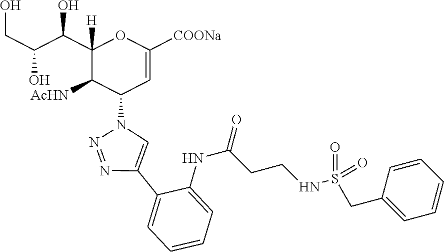

¹H NMR (400 MHz, D₂O): δ 1.90 (s, 3H), 2.57-2.66 (m, 2H), 3.36 (t, J=6.2 Hz, 2H), 3.63-3.75 (m, 2H), 3.92 (dd, J=12.0, 2.7 Hz, 1H), 4.03 (ddd, J=9.4, 6.3, 2.7 Hz, 1H), 4.40-4.52 (m, 3H), 4.57 (dd, J=10.9, 1.2 Hz, 1H), 5.56 (dd, J=9.7, 2.3 Hz, 1H), 5.86 (d, J=2.2 Hz, 1H), 7.40-7.48 (m, 6H), 7.51 (td, J=7.7, 1.7 Hz, 1H), 7.60 (dd, J=7.9, 1.4 Hz, 1H), 7.72 (dd, J=7.7, 1.7 Hz, 1H), 8.24 (s, 1H); ¹³C NMR (101 MHz, D₂O): δ 21.71, 37.06, 39.35, 48.72, 57.50, 59.99, 63.09, 68.09, 69.71, 75.38, 101.86, 122.36, 124.78, 126.65, 127.42, 128.74, 128.88, 129.30, 129.62, 130.70, 133.16, 144.96, 150.52, 168.69, 172.87, 173.60.

### Biology

### Cells and Virus:

LLC-MK2 cells (Rhesus monkey kidney, ATCC CCL-7) and MA104 cells (Rhesus monkey kidney, ATCC CRL-2378.1) were cultured in Eagle's minimal essential medium (EMEM) supplemented with 1% Glutamine (200 mM) and 2% of foetal bovine serum. During hPIV-3 (LLC-MK2) and hPIV-1 (MA104) infection and post-infection incubation, LLC-MK2 and MA104 cells were maintained in EMEM supplemented only with 1% glutamine. All cell lines were incubated at 37° C. in a humidified atmosphere of 5% CO₂. hPIV-3 (strain C-243) and hPIV-1 (strain C-35) were obtained from the American Type Culture Collection (ATCC). hPIV-3 (strain JS) was obtained from Viratree. hPIV-3 (strain C1002) is clinical isolate obtained from the Gold Coast University Hospital. The viruses were propagated in LLC-MK2 cells for hPIV-3 and in MA104 cells for hPIV-1 with EMEM supplemented with only glutamine at 35° C. in a humidified atmosphere of 5% CO₂. Virus-containing culture supernatant was collected 3 to 4 days post-infection, while monitoring cytopathic effects, and clarified from cell debris by centrifugation (3,000 RCF for 15 min). Virus was concentrated at least 10 times using 30 kDa Amicon Ultra filter unit for use in Haemagglutination Inhibition (HI) assays. Neuraminidase Inhibition (NI) assays used virus that was PEG-precipitated and then purified as described below. Clarified hPIV-3 or hPIV-1 supernatant was mixed with PEG6000 (8% final concentration) and NaCl (0.4 M final concentration) then incubated overnight at 4° C. under gentle agitation. PEG6000/hPIV complex was pelleted by centrifugation at 3,000 RCF for 30 min at 4° C. The supernatant was discarded and a volume of GNTE buffer (200 mM glycine, 200 mM NaCl, 20 mMTris-HCl, 2 mM EDTA, pH 7.4) corresponding to at least 1:40 of the initial virus suspension volume was used to resuspend the pellet overnight at 4° C. The virus suspension was homogenized by up and down pipetting followed by a mechanical disruption of the remaining virus aggregates using a douncer with “tight” pestle. The hPIV-3 or hPIV-1 homogenate was loaded on top of a 30%-60% non-linear sucrose gradient prepared in GNTE buffer and centrifuged at 100,000 RCF for 2 h 30 min at 4° C. without brake for deceleration. The virus was concentrated at the 30%-60% sucrose interface and then collected and stored at −4° C. for NI assays.

hPIV HN Inhibitors:

Compounds were provided as a lyophilized powder and then solubilized in sterile water or DMSO to generate a 10 mM stock solution. Solutions were sonicated for 15 min to allow complete dissolution. The stock solution was stored in an amber glass vial at −20° C. and freshly diluted in appropriate buffer before use.

### Haemagglutination Inhibition Assay:

The HN inhibitors were assessed in duplicate in a U-bottom 96 well plate assay. Compounds were diluted in PBS as a 4× solution for each concentration tested (25 μL/well, 1× final). Each dilution was mixed with 4 haemagglutination units (HAU) of hPIV-3 or hPIV-1 (25 μL/well, 1 HAU final) and incubated for 20 min at room temperature. An equivalent volume (50 μL/well) of 1% human red blood cells (h-RBC) was added to each well. The plate was then incubated for 1 h at room temperature (22-23° C.) before reading the extent of haemagglutination. The HI IC₅₀ considered as the concentration of inhibitor that reduced the haemagglutinin activity (agglutination) by 50% compared to those of a 1 HAU of non-treated virus suspension.

### Neuraminidase Inhibition Assay:

Purified hPIV-3 or hPIV-1, inhibitors and MUN were prepared and diluted in NA Reaction Buffer [NaOAc 50 mM, CaCl₂ 5 mM, pH 4.6 (hPIV3) or 5.0 (hPIV-1)]. Neuraminidase assay, employing different hPIV-3 or hPIV-1 dilutions, were initially measured to determine the lowest virus concentration to be used in the assays. The neuraminidase assays were performed with enough purified virus to obtain a maximal fluorescence signal at least 5 times higher than the background for the experiment to be considered statistically significant. Neuraminidase inhibition (NI) assays were done in triplicate. For each concentration tested, 2 μL of purified hPIV and 4 μL of 2.5× inhibitor solution (1× final) was added to each well. The plate was kept at room temperature for 20 min before 4 μL of 5 mM 2′-(4-Methylumbelliferyl)-α-D-N-acetylneuraminic acid (MUN) (2 mM final) was added to each well and then the plate incubated at 37° C. for 30 min with agitation (1100 rpm). The enzymatic reaction was stopped by the addition of 190 μl of glycine buffer (glycine 0.25 M, pH 10.4) to each well. A negative control was included by the addition of MUN to virus and then the enzymatic reaction stopped at t=0 min. Relative fluorescence (RF) was measured with a Tecan Infinite M200 Pro. Data were processed by background subtraction (negative control RF) and then analysed with GraphPadPrism to calculate IC₅₀ values (nonlinear regression (curve fit), Dose-response-inhibition, 3 or 4 parameter logistic). The concentration of inhibitor that reduced neuraminidase activity (relative fluorescence) by 50% compared to those of a non-treated virus suspension was considered to be the NI IC₅₀ value. All assays were performed in triplicate.

### In Situ Enzyme-Linked Immunosorbent Assay (ELISA):

In situ ELISA is a useful technique to evaluate virus growth inhibition. It measures, in one step, the expression level of hPIV-3 HN at the cell surface of an infected LLC-MK2 cell monolayer. The expression level is directly correlated to the ability of a non-immobilized virus to infect and re-infect target cells. Before assessing the best inhibitors in cell-based assays, an MTT assay can be performed to evaluate compound cytotoxicity. Infection was performed on a confluent LLC-MK2 cell monolayer seeded in a 96 well plate with 200 FFU/well. Infection with hPIV3 strains C243, JS or C1002 was done in triplicate and continued for 1 h at 37° C. with gentle agitation every 15 min. Compounds were diluted at a final concentration from 250 mM to 2.5 nM as a 10-fold dilution series. Inocula were removed and replaced with 100 μL/well of each respective compound dilution. A positive control for infection was incorporated by the use of identical experimental conditions, minus inhibitor. Infected cell monolayers were kept for 36-40 h at 37° C. 5% CO₂ for virus proliferation. Virus was inactivated and cells fixed by the direct addition of 100 μL of 7.4% formaldehyde/PBS. The plate was maintained at room temperature for 15 min and then washed 3 times for 5 min with PBS and then endogenous peroxidases were inactivated by treatment with 0.3% H₂O₂/PBS for 30 min at 37° C. The cell monolayers were washed and incubated with mouse monoclonal IgG anti-hPIV-3HN (Fitzgerald, clone #M02122321, 2.0 mg/mL) at 1 μg/mL in 5% milk/PBS for 1 h at 37° C. The wells were washed 3 times for 5 min with 0.02% Tween20/PBS. Goat anti-Mouse-IgG(H+L)-HRP conjugate (BioRad, #1706516), diluted at 1:4000 in 5% milk/PBS, was added to each well and incubated for 1 h at 37° C. Cell monolayers were washed with 0.02% Tween20/PBS and then rinsed twice with PBS. BD OptEIA TMB substrate was added to each well and the plate was then incubated at 37° C. The enzymatic reaction was stopped after 3-5 min by the addition of 50 μL of 0.6 M of H₂SO₄ per well. Raw data were obtained by reading the absorbance (OD) of each well at 450 nm using a xMark™ Microplate Absorbance Spectrophotometer. Final ODs were obtained by subtraction of the negative control (non-infected cells) OD from the initial OD reading and the data analysed with GraphPad Prism4 to calculate IC₅₀ values (nonlinear regression (curve fit), Dose response-inhibition, 3 or 4 parameter logistic). The IC₅₀ value was considered as the concentration of inhibitor that reduced the absorbance at 450 nm by 50%, compared to a non-treated infected cell monolayer.

Compounds of the present invention can be tested in a hPIV-3 inhibition assay on ex vivo differentiated human airway epithelial (HAE) cells using a published model. In brief the testing procedure is as follows: Human airway epithelial (HAE) cells are isolated, cultured and differentiated as previously described (Muller et al., 2013). Briefly, human nasal airway epithelial cells are isolated, expanded and seeded on collagen-coated permeable membrane supports (transwell). Once the cells are confluent, the apical medium is removed, and cells are maintained at the air-liquid interface for approximately 4 to 6 weeks to allow epithelial differentiation. Cultures containing ciliated cells are inoculated via the apical surface with 400 focus forming units of hPIV-3 per transwell for 1 hour. Test compounds at various concentrations are added to HAE apical side (20 μL/transwell) just after the cells have been infected for 1 h with the virus. Viral load reduction is assessed at 1, 3 and 6 days post-infection by virus titration using focus forming assay or in situ ELISA in LLC-MK2 cells, as previously published (Guillon et al., 2014).

### Structural Biology

### Recombinant HN Expression and Purification

The HN protein was expressed using the Bac-to-Bac® baculovirus expression system (Invitrogen, Carlsbad, Calif.) based on a substantially modified literature procedure. Thus, the nucleotide sequence for a honeybee melittin signal peptide (HBM) was added downstream to the sequence encoding for the HN ectodomain (amino acids 125 to 572). This sequence (HBM+HN) was codon optimised for expression in Spodoptera frugiperda cells (Sf9) and ordered directly through the DNA2.0 gene synthesis service (DNA2.0, Menlo Park, Calif.) as a gene named HBM-HNhPIV-3ₒₚₜ. HBM-HNhPIV-3ₒₚₜ was amplified by PCR and ligated into a pFastBac/CT-TOPO® vector that provides an additional C-terminal 6-histidine tag (His-Tag) for purification and detection purposes.

The generation and amplification of recombinant baculovirus containing HBM-HNhPIV-3ₒₚₜ were performed according to the manufacturer's instructions. Sf9 cells (Invitrogen), cultured in Insect-XPRESS protein free insect cell medium (Lonza), were infected with high MOI of HBM-HNhPIV-3ₒₚₜ baculovirus. Four days post-infection the supernatant, containing recombinant HN, was collected to yield the highest protein expression. The supernatant was clarified by centrifugation (3,000 RCF for 15 min) to remove cell debris and then purified on a HisTrap excel 5 mL column (GE Healthcare life sciences, Buckinghamshire, England) following the manufacturer's protocol. Recombinant HN was eluted with 500 mM imidazole solution and collected fractions were assessed for their neuraminidase enzymatic activity (see above). The most active fractions were pooled and concentrated with a 10 kDa Amicon Ultra filter unit (Millipore) to a final volume of 800 μL. An additional purification step was performed that employed fast protein liquid chromatography (Amersham Biosciences) over a Superdex 75 gel filtration column (GE Healthcare) at 4° C. and 1 mL fractions were collected with a Frac-920. Protein-containing fractions, as determined by monitoring fraction collection at 280 nm, were assessed for their neuraminidase enzymatic activity as well as subjected to SDS-PAGE. Purified and concentrated recombinant HN protein was stored at 4° C.

### Crystallisation, Data Collection and Structure Determination

Some of the hPIV3-HN complexes (with compound IE-1826.23) were prepared by soaking crystals in a crystallisation solution (0.1 M citrate buffer pH 4.6, 0.2 M (NH₄)2SO₄, 15% v/v Polyethylene glycol (PEG) 3000) containing 5 mM of inhibitors for various times between 1 hr-24 hrs. Other hPIV-3 HN complexes were prepared by co-crystallisation (with compounds IE-1826.30, IE-1530.74, IE-1530.69 and IE-1778.39) where the 4 mg/mL hPIV3 HN protein stock solution was preincubated with a final concentration of 1.5 mM inhibitor in 0.1 M citrate buffer pH 4.6, 0.2 M (NH₄)₂SO₄ and 10% PEG 3000 for 30 min. Crystallization trials were set up as 2 μL preincubated stock solution using the hanging drop vapour diffusion method. The drop was equilibrated against a 500 μL reservoir (0.1 M citrate buffer pH 4.6, 0.2 M (NH₄)₂SO₄ and 10% or 15% PEG 3000). The crystals were mounted in nylon loops (Hampton Research) and flash frozen at 100 K in a cryoprotectant solution containing 20% glycerol in addition to the precipitant solution.

X-ray diffraction data were collected on the MX2 beamline at the Australian Synchrotron using the Blu-Ice software. The datasets were processed using XDS and scaled using Aimless in the CCP4 suite. The structures were solved by molecular replacement using Phaser and the apo hPIV3-HN model (PDB ID: 4XJQ) as template. The models were refined using Phenix. Refine, and structure validation was performed using MolProbity. Structure analyses were performed using Coot, and PyMOL (http://www.pymol.org/; DeLano Scientific LLC).

### Results

TABLE 1
Biological evaluation on hPIV-3 strain C243 of the inhibitor examples.

| | | | In situ |
|---|---|---|---|
| | | NI | ELISA |
| Cmpd | | (IC₅₀, | (IC₅₀, |
| no. | Structure | μM) | μM) |
| IE1778- 64 | 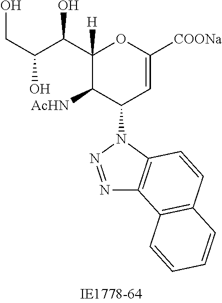 | 4.133 | Not tested |
| IE1778- 74 | 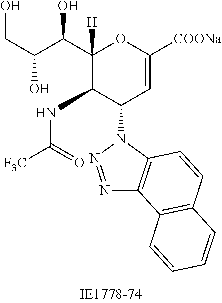 | 0.5677 | 1.87 |
| IE1530- 66 | 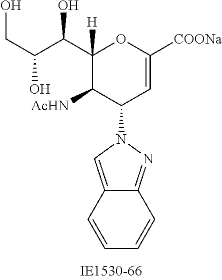 | 92.81 | Not tested |
| IE1530- 74 | 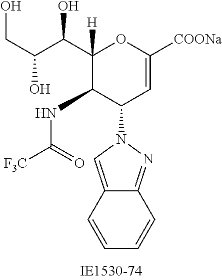 | 9.37 | Not tested |
| IE1530- 65 | 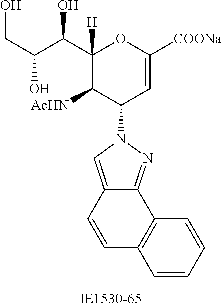 | 10.25 | Not tested |
| IE1530- 69 | 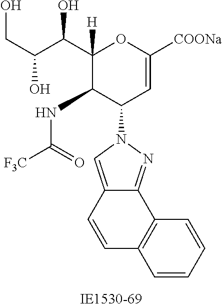 | 2.837 | Not tested |
| IE1778- 12 | 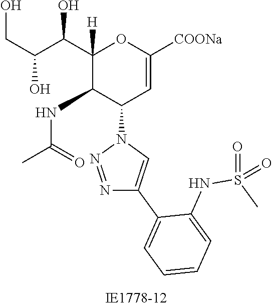 | 29.08 | Not tested |
| IE1778- 25 | 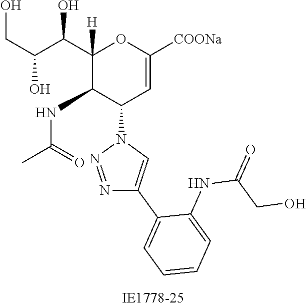 | 27.95 | Not tested |
| IE1826- 23 | 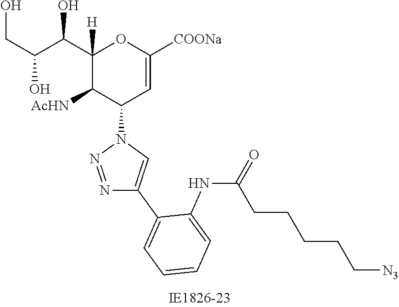 | 69.18 | Not tested |
| IE1826- 30 | 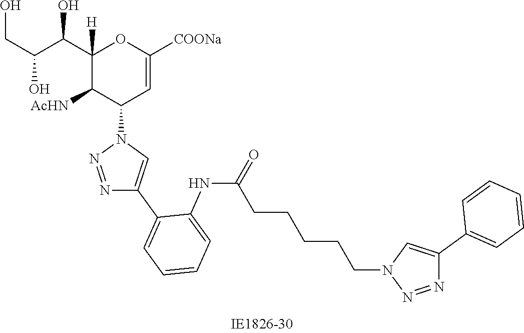 | 24.3 | Not tested |
| IE1826- 34 | 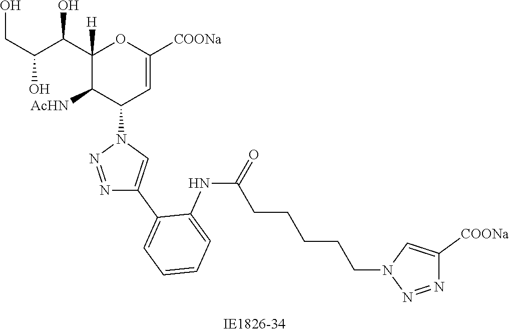 | 16.74 | Not tested |
| IE1826- 38 | 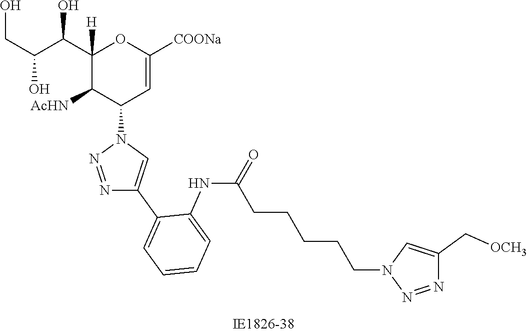 | 24.18 | Not tested |
| IE1826- 44 | 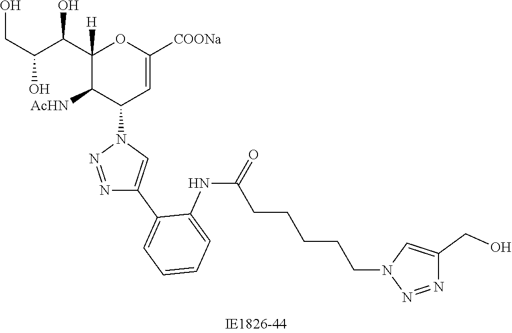 | 47.25 | Not tested |
| IE1826- 01 | 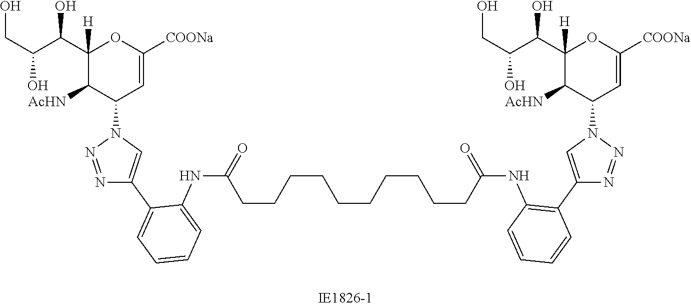 | 8.095 | 17.93 |
| IE1826- 14 | 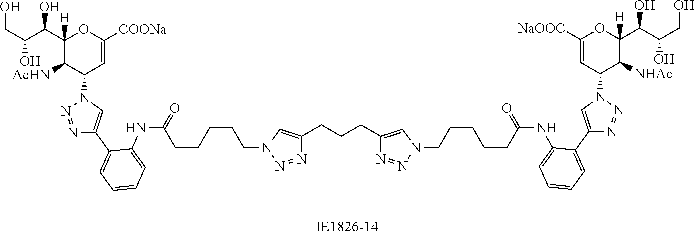 | 8.217 | Not tested |
| IE1826- 28 | 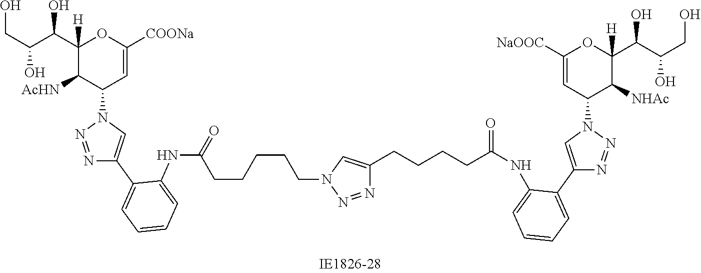 | 13.98 | Not tested |

NI: neuraminidase inhibition.

TABLE 2
Biological evaluation of the inhibitor examples on hPIV-3 (strains C243, JS, CI002) and hPIV-1 (strain C35).

| | | | In situ ELISA |
|---|---|---|---|
| Structure | HI IC₅₀ (μM) | NI IC₅₀ (μM) | IC₅₀ (μM) |
| 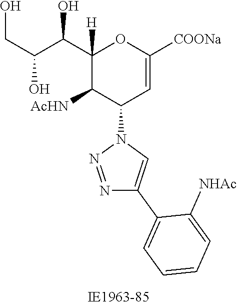 | >200 (hPIV1 C35) >200 (hPIV3 CI002) 40 (hPIV3 JS) | — | 126 (hPIV3 C243) 406 (hPIV3 JS) |
| 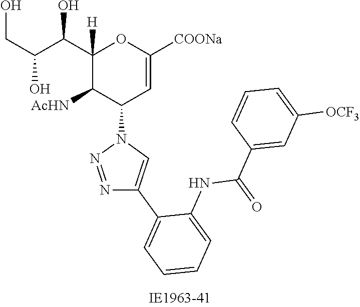 | >200 (hPIV3 C243) >200 (hPIV3 CI002) >250 (hPIV3 JS) | 47.37 (hPIV3 C243) | 217 (hPIV3 C243) >500 (hPIV3 JS) |
| 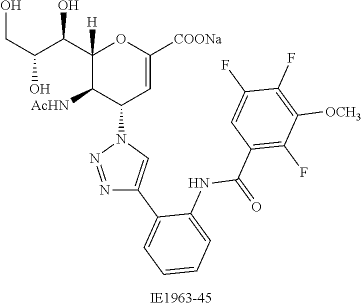 | >200 (hPIV1 C35) (hPIV3 CI002) >250 (hPIV3 JS) | 32.82 (hPIV3 C243) | 176.6 (hPIV3 C243) 764.5 (hPIV3 JS) |
| 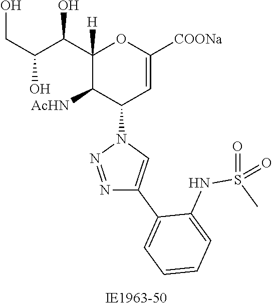 | >200 (hPIV1 C35) 60 (hPIV3 CI002) 28 (hPIV3 JS) | — | — |
| 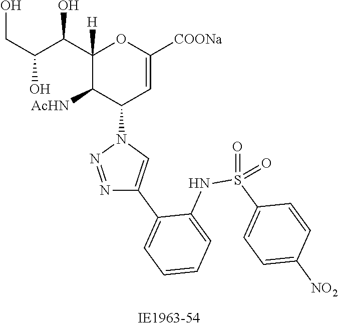 | >200 (hPIV1 C35) 2.56 (hPIV3 C243) 13.7 (hPIV3 CI002) 4.48 (hPIV3 JS) | — | 29.12 (hPIV3 CI002) |
| 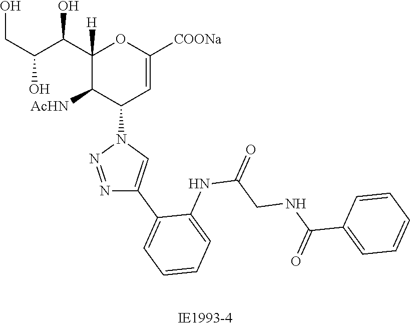 | >200 (hPIV1 C35) >200 (hPIV3 CI002) 20 (hPIV3 JS) | — | 340 (hPIV3 JS) |
| 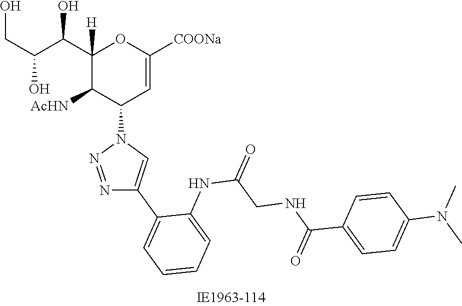 | >200 (hPIV1 C35) >200 (hPIV3 CI002) 14 (hPIV3 JS) | — | 355 (hPIV3 JS) |
| 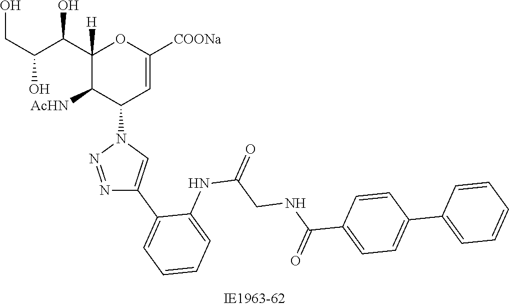 | >200 (hPIV1 C35) 44.5 (hPIV3 CI002) 6.4 (hPIV3 JS) | — | 23.73 (hPIV3 C243) 60.73 (hPIV3 JS) |
| 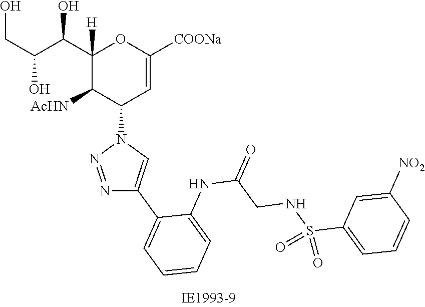 | >200 (hPIV1 C35) 44.5 (hPIV3 CI002) 15.4 (hPIV3 JS) | — | 99.06 (hPIV3 CI002) |
| 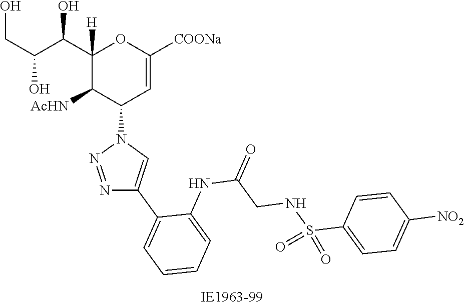 | >200 (hPIV1 C35) 23.9 (hPIV3 CI002) 8 (hPIV3 JS) | — | >200 (hPIV1 C35) 63.9 (hPIV3 C243) 240 (hPIV3 JS) |
| 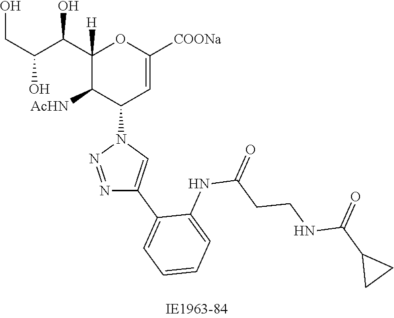 | >200 (hPVI3 C243) >200 (hPIV3 CI002) >250 (hPIV3 JS) | 30.17 (hPIV3 C243) | 104 (hPIV3 C243) 602 (hPIV3 JS) |
| 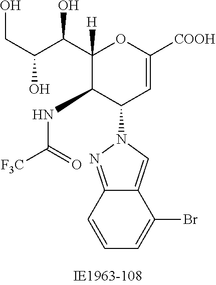 [SMILES: O=C(O)C1=C[C@H](n2cc3c(Br)cccc3n2)[C@@H](NC(=O)C(F)(F)F)[C@H]([C@H](O)[C@H](O)CO)O1] | 4.94 (hPIV1 C35) 7.41 (hPIV3 CI002) 4.94 (hPIV3 JS) | 3.95 (hPIV3 C243) 8.87 (hPIV3 CI002) | — |
| 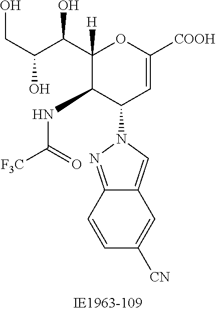 [SMILES: N#Cc1ccc2nn([C@H]3C=C(C(=O)O)O[C@@H]([C@H](O)[C@H](O)CO)[C@@H]3NC(=O)C(F)(F)F)cc2c1] | 66.7 (hPIV1 C35) >200 (hPIV3 CI002) 133 (hPIV3 JS) | 47.9 (hPIV3 C243) 143 (hPIV3 CI002) | — |
| 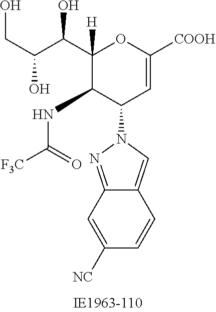 [SMILES: N#Cc1ccc2cn([C@H]3C=C(C(=O)O)O[C@@H]([C@H](O)[C@H](O)CO)[C@@H]3NC(=O)C(F)(F)F)nc2c1] | >200 (hPIV1 C35) >200 (hPIV3 CI002) >200 (hPIV3 JS) | 49.8 (hPIV3 C243) 208 (hPIV3 CI002) | — |
| 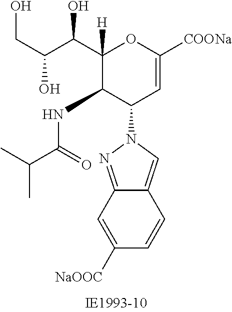 | >200 (hPIV1 C35) >200 (hPIV3 CI002) >200 (hPIV3 JS) | 211 (hPIV3 C243) 378 (hPIV3 CI002) | — |
| 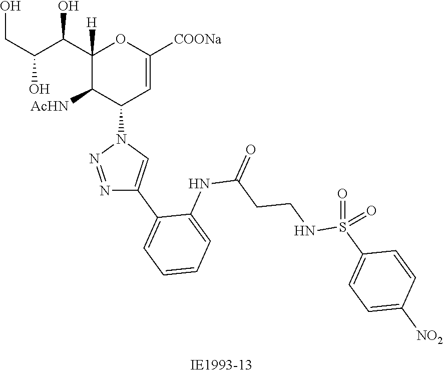 | >200 (hPIV1 C35) >200 (hPIV3 CI002) 38.5 (hPIV3 JS) | — | 287 (hPIV3 CI002) |
| 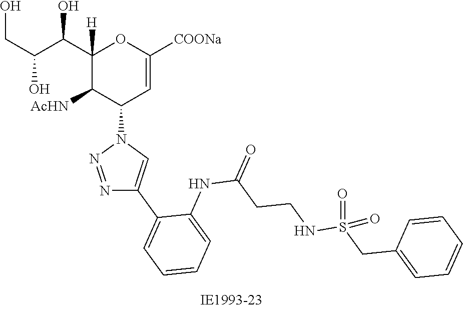 | >200 (hPIV1 C35) >200 (hPIV3 CI002) >200 (hPIV3 JS) | 21.2 (hPIV3 C243) 53.9 (hPIV3 CI002) | — |
| 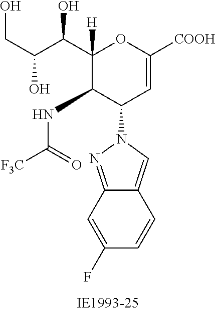 [SMILES: O=C(O)C1=C[C@H](n2cc3ccc(F)cc3n2)[C@@H](NC(=O)C(F)(F)F)[C@H]([C@H](O)[C@H](O)CO)O1] | 133 (hPIV1 C35) 22.2 (hPIV3 CI002) 22.2 (hPIV3 JS) | 12.9 (hPIV3 C243) 103 (hPIV3 CI002) | — |
| 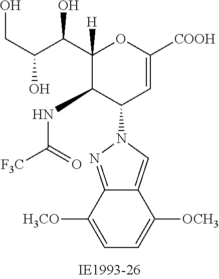 [SMILES: COc1ccc(OC)c2nn([C@H]3C=C(C(=O)O)O[C@@H]([C@H](O)[C@H](O)CO)[C@@H]3NC(=O)C(F)(F)F)cc12] | 14.8 (hPIV3 243) | 11.2 (hPIV3 C243) 74.2 (hPIV3 CI002) | — |
| 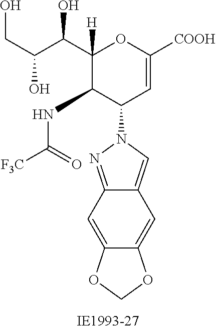 [SMILES: O=C(O)C1=C[C@H](n2cc3cc4c(cc3n2)OCO4)[C@@H](NC(=O)C(F)(F)F)[C@H]([C@H](O)[C@H](O)CO)O1] | 133 (hPIV1 C35) 14.8 (hPIV3 C243) 66.7 (hPIV3 CI002) | 12.9 (hPIV3 C243) 38.9 (hPIV3 CI002) | — |
| 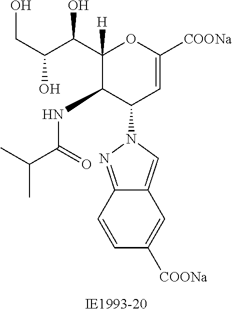 | >200 (hPIV3 C243) | 230 (hPIV3 C243) 163 (hPIV3 CI002) | — |

HI: haemagglutination inhibition.
NI: neuraminidase inhibition.
IC₅₀: concentration of inhibitor reducing by 50% the viral function/growth compared to experiment positive control (no inhibitor present).
hPIV1 strain: C35.
hPIV3 strains: C243, JS, CI002 (clinical isolate).

1. A compound of formula (I), or a pharmaceutically acceptable salt thereof: 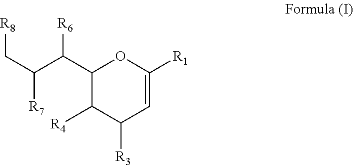
wherein, R₁ is selected from the group consisting of COOH, or a salt thereof, C(O)NR₉R₁₀, C(O)OR₁₁ wherein R₉, R₁₀ and R₁₁ are independently selected from the group consisting of hydrogen, optionally substituted C₁-C₆ alkyl and optionally substituted aryl;
R₃ is selected from the group consisting of optionally substituted N-linked naphthotriazole, optionally substituted N-linked indazole, and N-linked triazole of the following formula: 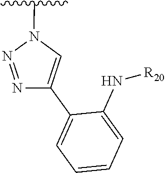
wherein R₂₀ is selected from the group consisting of 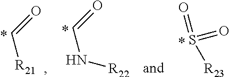
wherein, * is the point of attachment, R₂₁, R₂₂ and R₂₃ are independently selected from the group consisting of optionally substituted alkyl, optionally substituted alkenyl, substituted alkynyl, optionally substituted cycloalkyl, optionally substituted aryl, optionally substituted heteroaryl, optionally substituted heterocyclyl, optionally substituted alkylheterocyclic, optionally substituted alkylheteroaryl, optionally substituted alkylamine, optionally substituted dialkylamine and an optionally substituted linker which links the compound to another compound of Formula (I);
R₄ is selected from the group consisting of sulfonamide, urea and NHC(O)R₁₇ wherein R₁₇ is selected from the group consisting of C₁-C₆ alkyl, C₁-C₆ haloalkyl and C₃-C₆ cycloalkyl, all of which may be optionally substituted;
R₆, R₇ and R₈ are independently selected from the group consisting of OH, protected OH, NH₂, C₁-C₆ alkyl, C₁-C₆ haloalkyl, NR₁₈R₁₈′, C₁-C₆ alkoxy, C₁-C₆ haloalkoxy, —OC(O)R₁₈, —NH(C═O)R₁₈, and S(O)ₙR₁₈, wherein n=0-2 and each R₁₈ and R₁₈′ are independently selected from hydrogen, optionally substituted C₁-C₆ alkyl and optionally substituted C₁-C₉ alkanoyl, as appropriate.

2. The compound of claim 1 wherein R₁ is COOH, or a salt thereof, or C(O)OR₁₁ wherein R₁₁ is selected from methyl, ethyl and propyl.

3. The compound of claim 1 wherein when R₃ is optionally substituted N-linked naphthotriazole it is of the following formula: 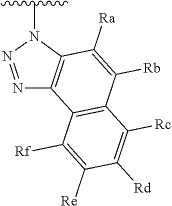
wherein, Rₐ, R_{b}, R_{c}, R_{d}, Rₑ, and R_{f} are independently selected from the group consisting of hydrogen, hydroxyl, cyano, halo, amido, C₁-C₁₂ alkyl, C₁-C₁₂ alkoxy, C₁-C₁₂ haloalkoxy, C₁-C₁₂ alkanoyl, C₁-C₁₂ haloalkanoyl, C₁-C₁₂ haloalkyl, pyridyl and phenyl, all of which may be optionally substituted as appropriate.

4. The compound of claim 1 wherein when R₃ is optionally substituted N-linked indazole it is of the following formula: 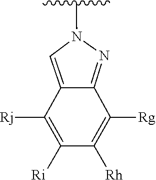
wherein, R_{g}, Rₕ, Rᵢ, and Rⱼ are independently selected from the group consisting of hydrogen, hydroxyl, halo, C₁-C₆ alkoxy, C₁-C₆ haloalkyl, cyano, sulfonyl, amine, alkylamine, dialkylamine, amido, and carboxyl; and
R_{g} and Rₕ, Rₕ and Rᵢ, and Rᵢ and Rⱼ may together form a heteroaryl, heterocyclic or aryl ring, each of which may be optionally substituted.

5. The compound of claim 1 wherein when R₃ is N-linked triazole, as defined in claim 1, R₂₁ may be selected from the group consisting of optionally substituted C₁-C₉ alkyl, optionally substituted C₂-C₉ alkenyl, optionally substituted 5 or 6 membered aryl, optionally substituted C₁-C₉ alkyl-nitrogenheterocycle, optionally substituted C₁-C₉ alkyl-nitrogenheteroaryl, optionally substituted C₁-C₉ alkylamine, optionally substituted C₁-C₆ alkyl-NH—CO-aryl, optionally substituted C₁-C₆ alkyl-NH—CO-aryl-aryl, optionally substituted C₁-C₆ alkyl-NH—CO-cycloalkyl, optionally substituted C₁-C₆ alkyl-NH—SO₂-aryl, optionally substituted C₁-C₆ alkyl-NH—SO₂—C₁-C₆alkyl-aryl and an optionally substituted linker which links the compound to another compound of Formula (I).

6. The compound of claim 1 wherein when R₃ is N-linked triazole, as defined in claim 1, when R₂₁ is an optionally substituted linker which links the compound to another compound of Formula (I) then the compound of formula (I) is of the following formula: 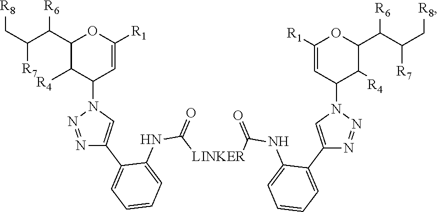
wherein, R₁, R₄, R₆, R₇ and R₈ are as defined in claim 1 and LINKER is selected from C₁-C₁₂ alkyl; C₁-C₉ alkyl; C₂-C₉ alkenyl; and C₂-C₉ alkynyl; which are all optionally substituted and optionally linked to a 5-membered nitrogen heteroaryl.

7. The compound of claim 1 wherein R₃ is selected from the group consisting of:

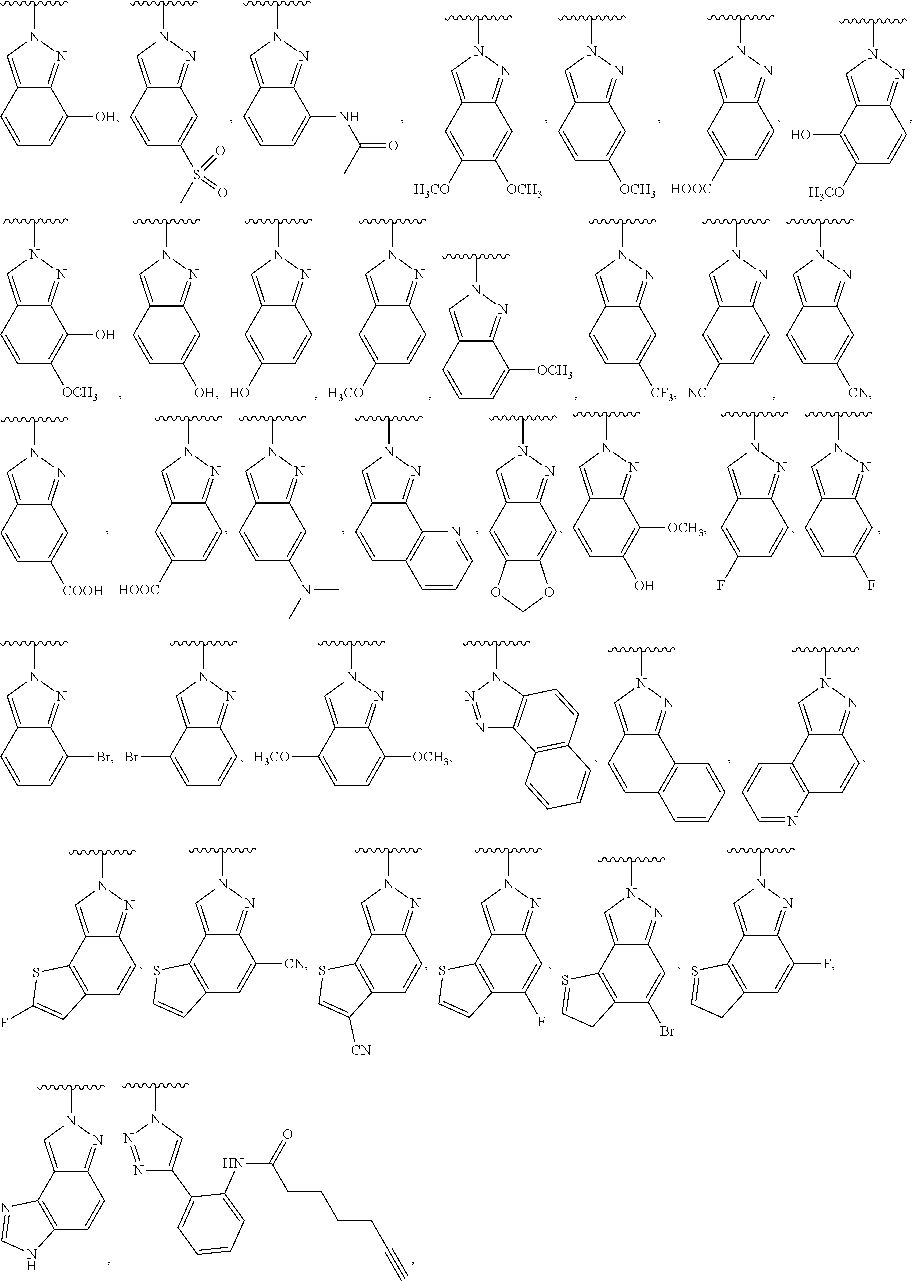

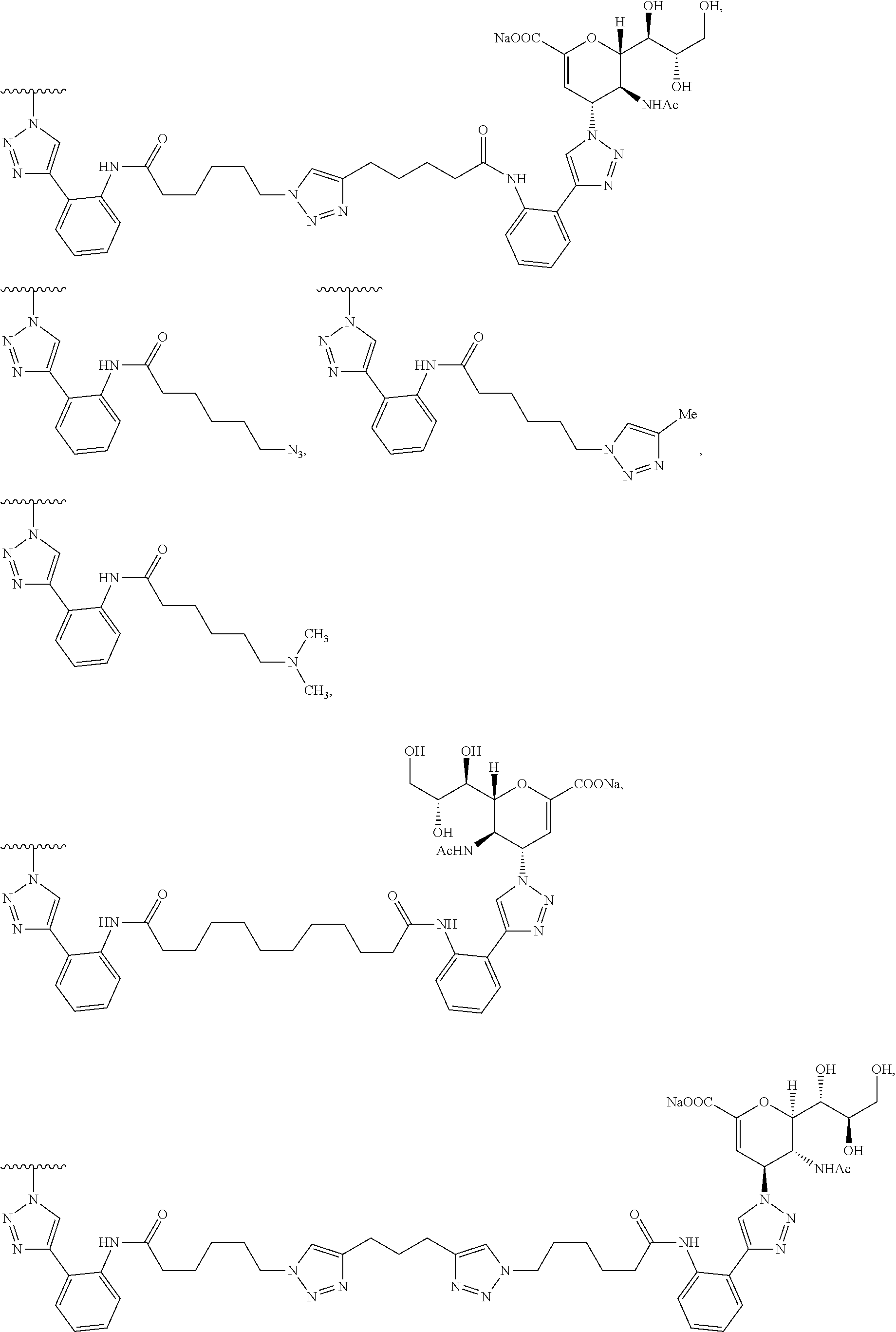

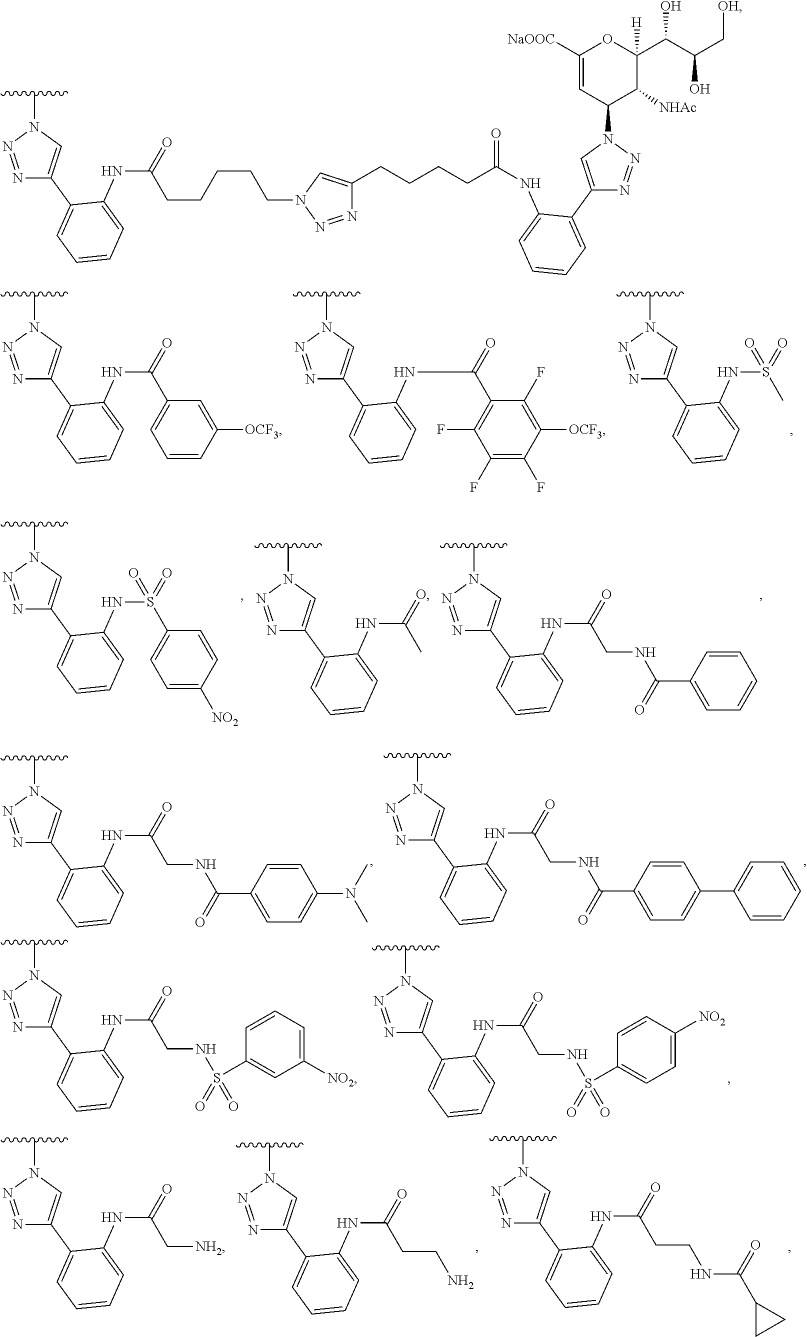

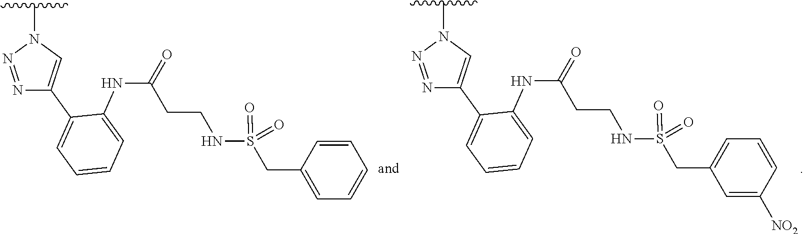 [SMILES: *n1cc(-c2ccccc2NC(=O)CCNS(=O)(=O)Cc2ccccc2)nn1 ; *n1cc(-c2ccccc2NC(=O)CCNS(=O)(=O)Cc2cccc([N+](=O)[O-])c2)nn1]

8. The compound of claim 1 wherein R₄ is selected from the group consisting of —NHS(O)₂R₂₇ wherein R₂₇ is selected from the group consisting of C₁-C₆ alkyl, C₁-C₆ haloalkyl and C₃-C₆ cycloalkyl, all of which may be optionally substituted; —NHC(O)NHR₁₇, wherein R₁₇ is selected from the group consisting of C₁-C₆ alkyl, C₁-C₆ haloalkyl and C₃-C₆ cycloalkyl, all of which may be optionally substituted; and the following:

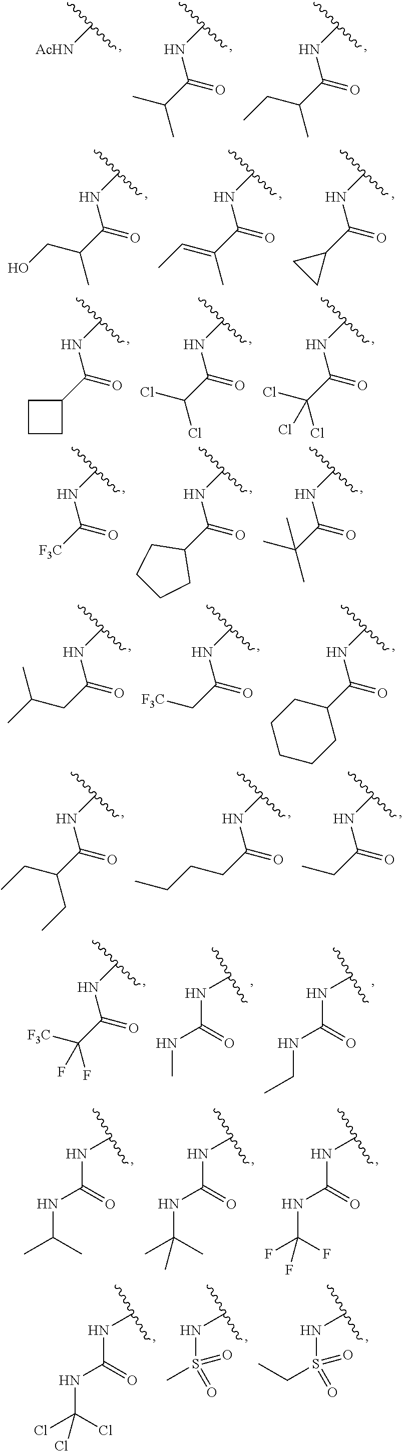

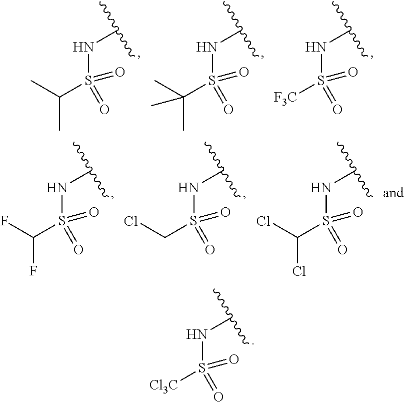

9. The compound of claim 1 wherein R₄ is selected from the group consisting of —NHAc, —NHC(O)CH(CH₃)₂, —NHC(O)CF₃ and —NHC(O)CH₂CH₃.

10. The compound of claim 1 wherein R₆, R₇ and R₈ are independently selected from OH and OAc.

11. The compound of claim 1 wherein the compound of formula (I) is a compound of formula (II): 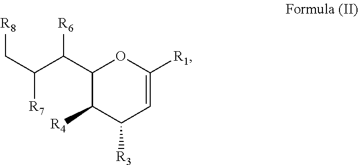
wherein, R₁, R₃, R₄, R₆, R₇ and R₈ are as described in claim 1.

12. The compound of claim 1 wherein the compound is a compound of formula (IIIa) or (IIIb): 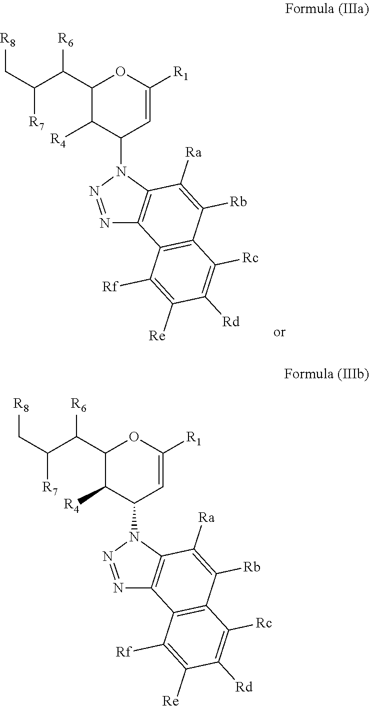
wherein, R₁, R₄, R₆, R₇, and R₈, are as defined in claim 1, and Rₐ, R_{b}, R_{c}, R_{d}, Rₑ, and R_{f} are independently selected from the group consisting of hydrogen, hydroxyl, cyano, halo, amido, C₁-C₁₂ alkyl, C₁-C₁₂ alkoxy, C₁-C₁₂ haloalkoxy, C₁-C₁₂ alkanoyl, C₁-C₁₂ haloalkanoyl, C₁-C₁₂ haloalkyl, pyridyl and phenyl, all of which may be optionally substituted as appropriate.

13. The compound of claim 1 wherein the compound is a compound of formula (IVa) or (IVb): 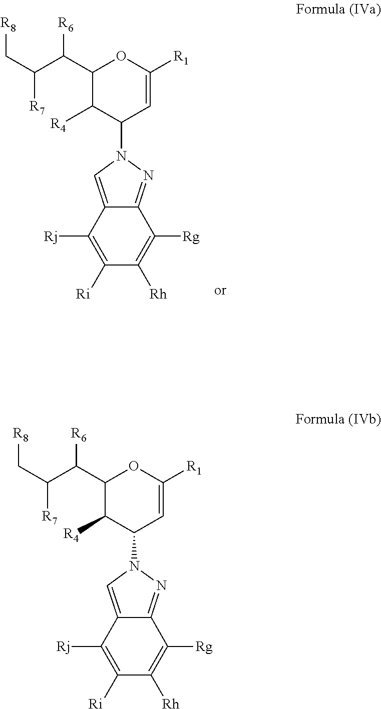
wherein, R₁, R₄, R₆, R₇, and R₈ are as defined in claim 1, and R_{g}, Rₕ, Rᵢ, and Rⱼ are independently selected from the group consisting of hydrogen, hydroxyl, halo, C₁-C₆ alkoxy, C₁-C₆ haloalkyl, cyano, sulfonyl, amine, alkylamine, dialkylamine, amido, and carboxyl; and
R_{g} and Rₕ, Rₕ and Rᵢ, and Rᵢ and R₁ may together form a heteroaryl, heterocyclic or aryl ring, each of which may be optionally substituted.

14. The compound of claim 1 wherein the compound is a compound of any one or more of formulae Va, Vb, VIa, VIb, VIIa and VIIb: 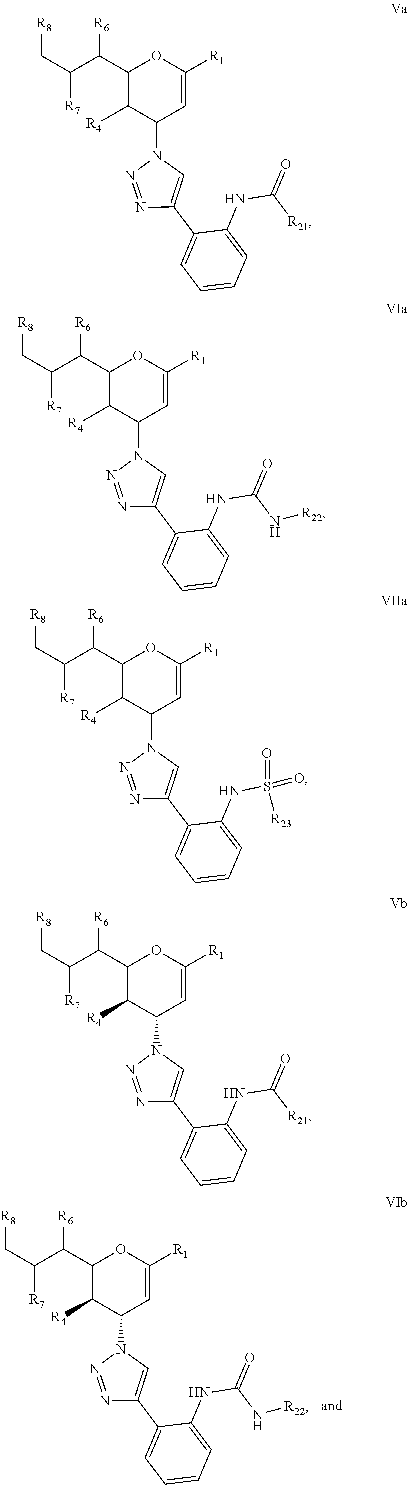 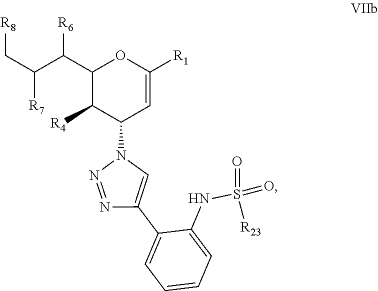
wherein, R₁, R₄, R₆, R₇, R₈, R₂₁, R₂₂ and R₂₃ are as defined in claim 1.

15. The compound of claim 1 wherein the compound of formula (I) is a compound selected from the group consisting of: 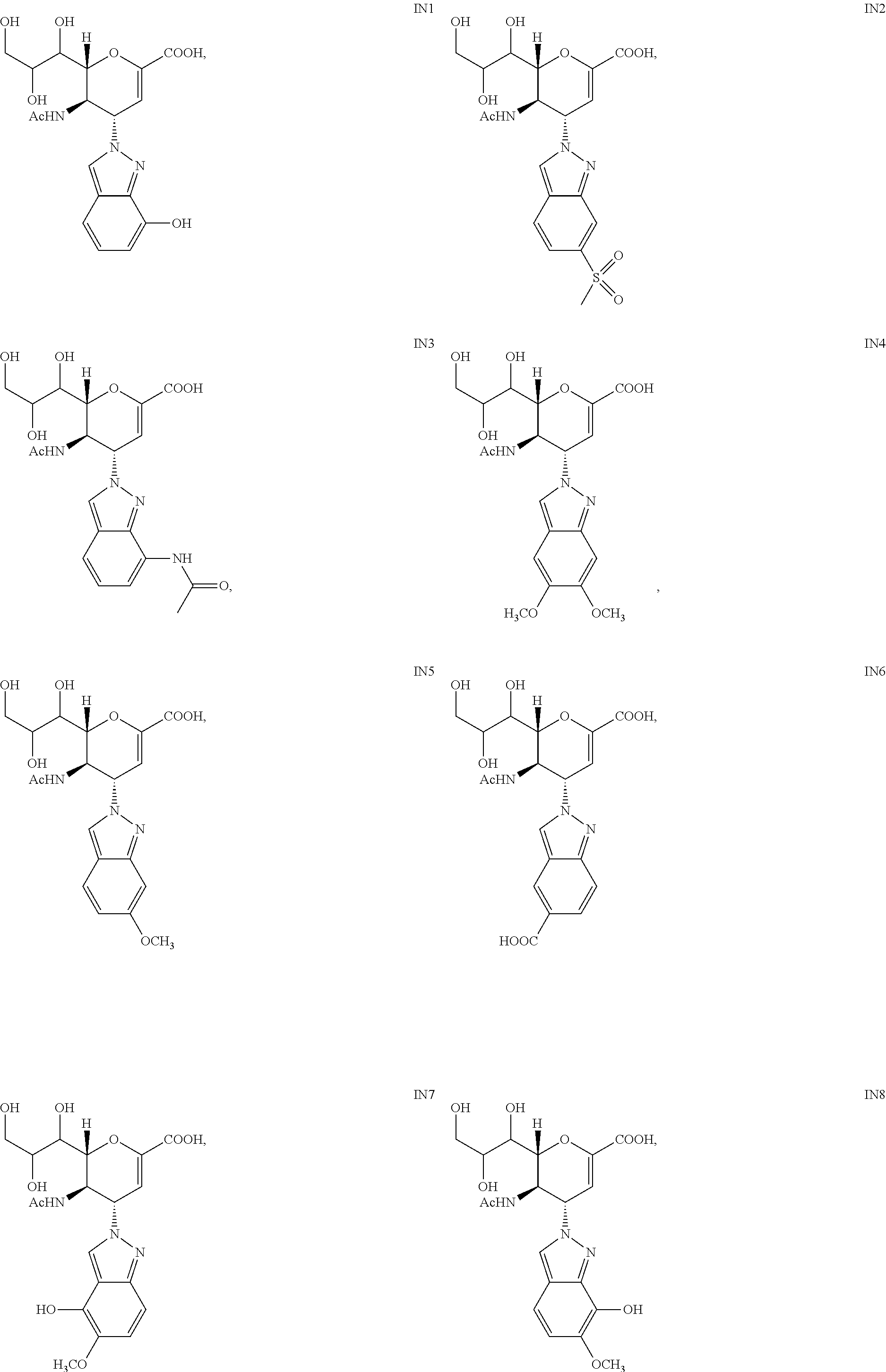 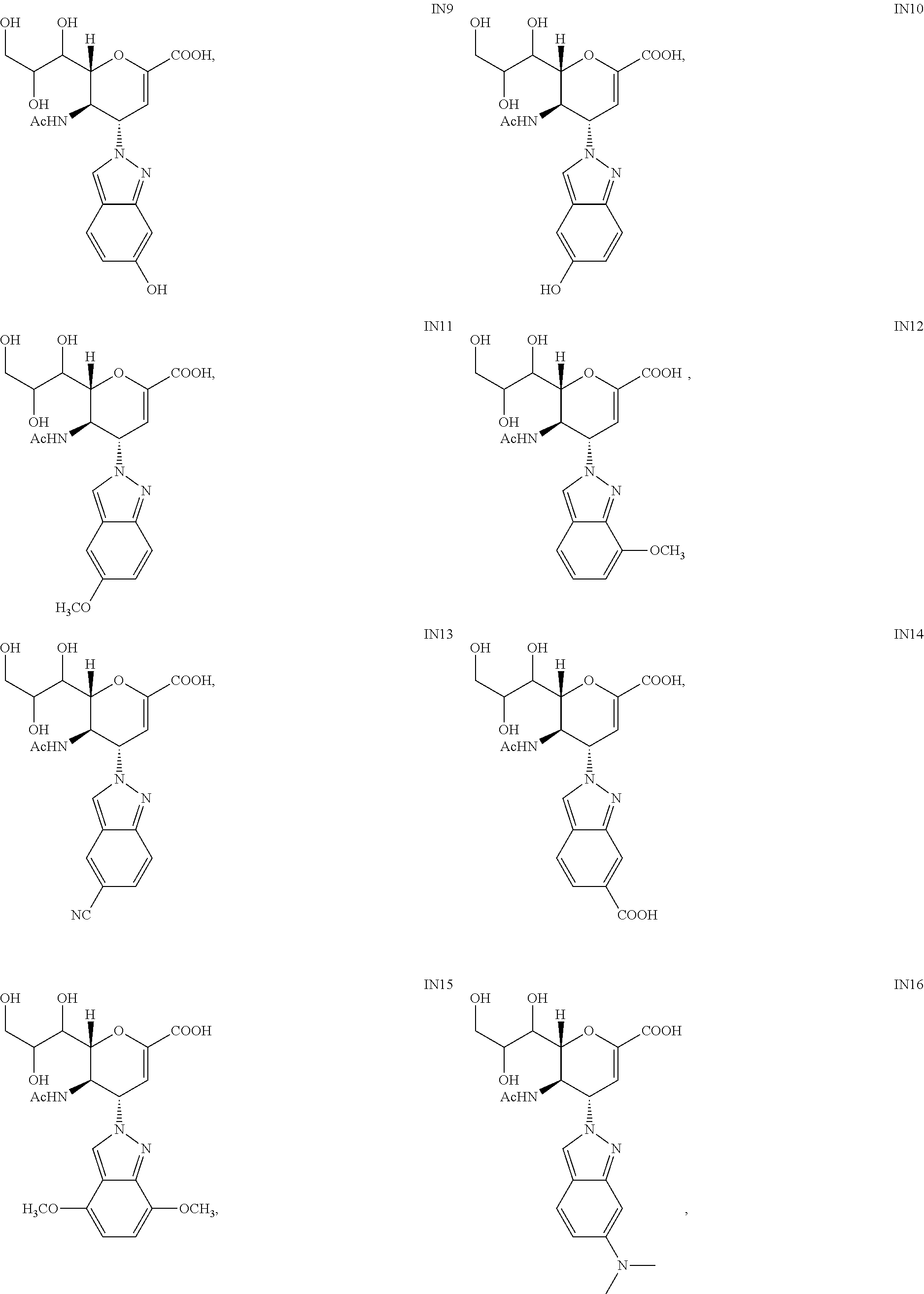 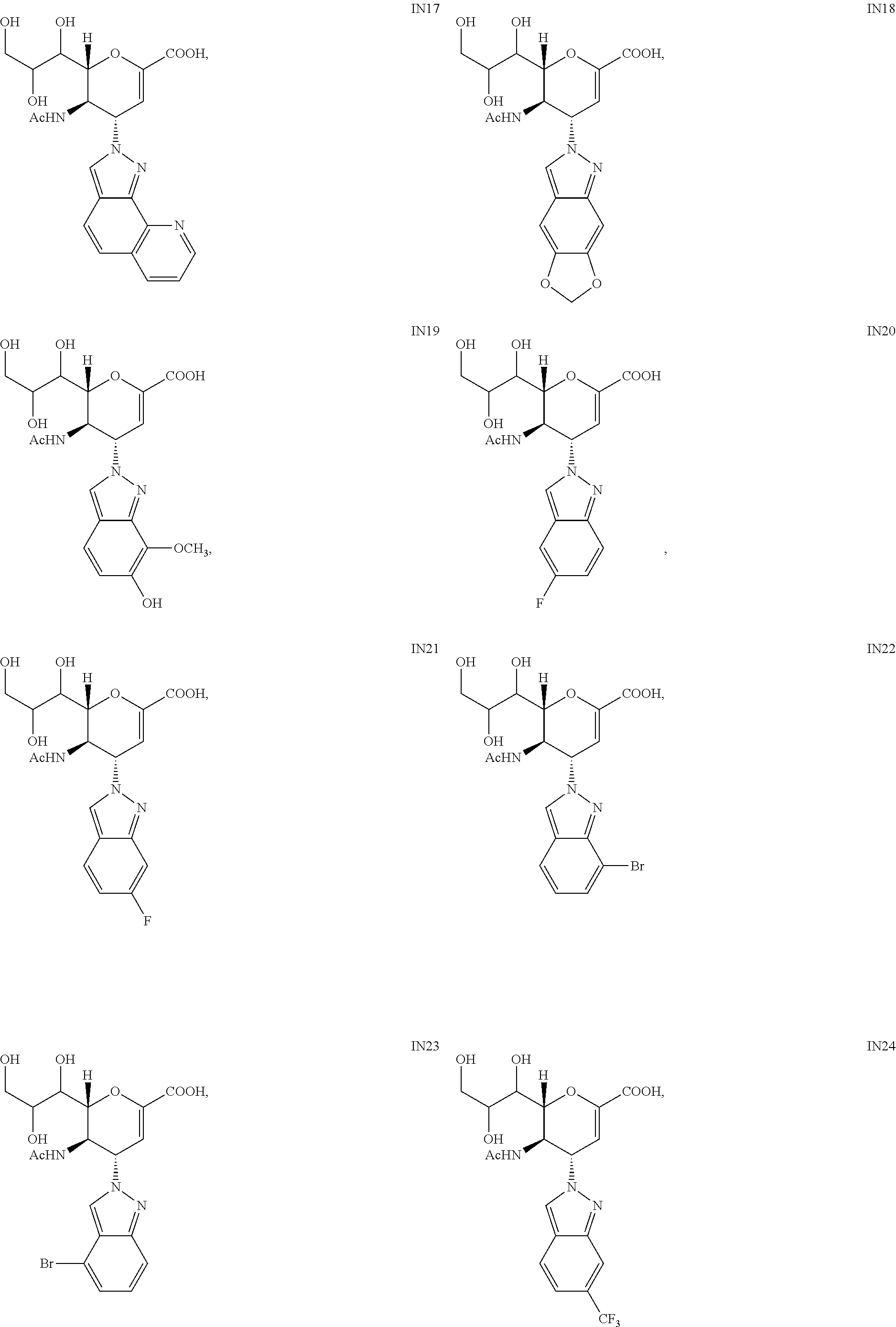 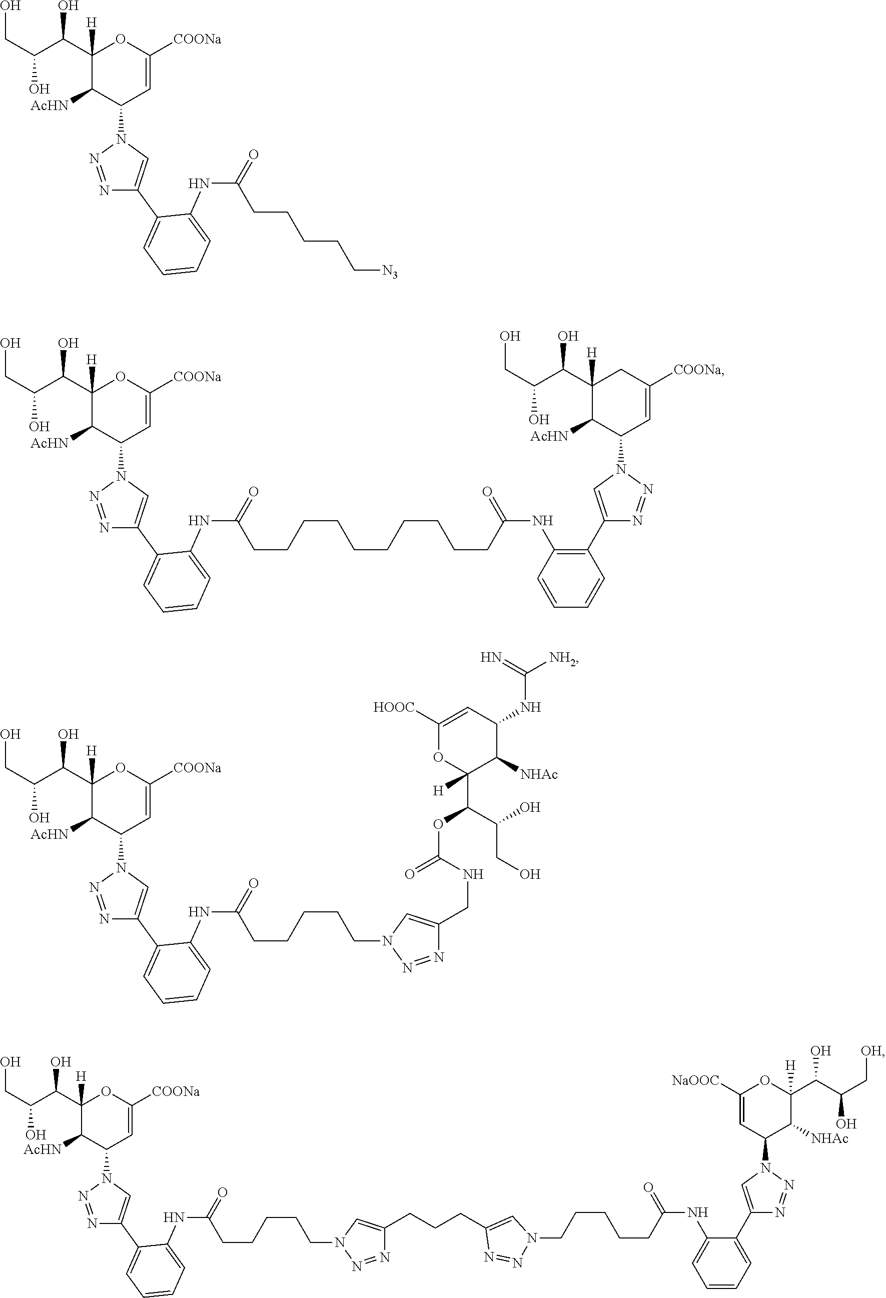 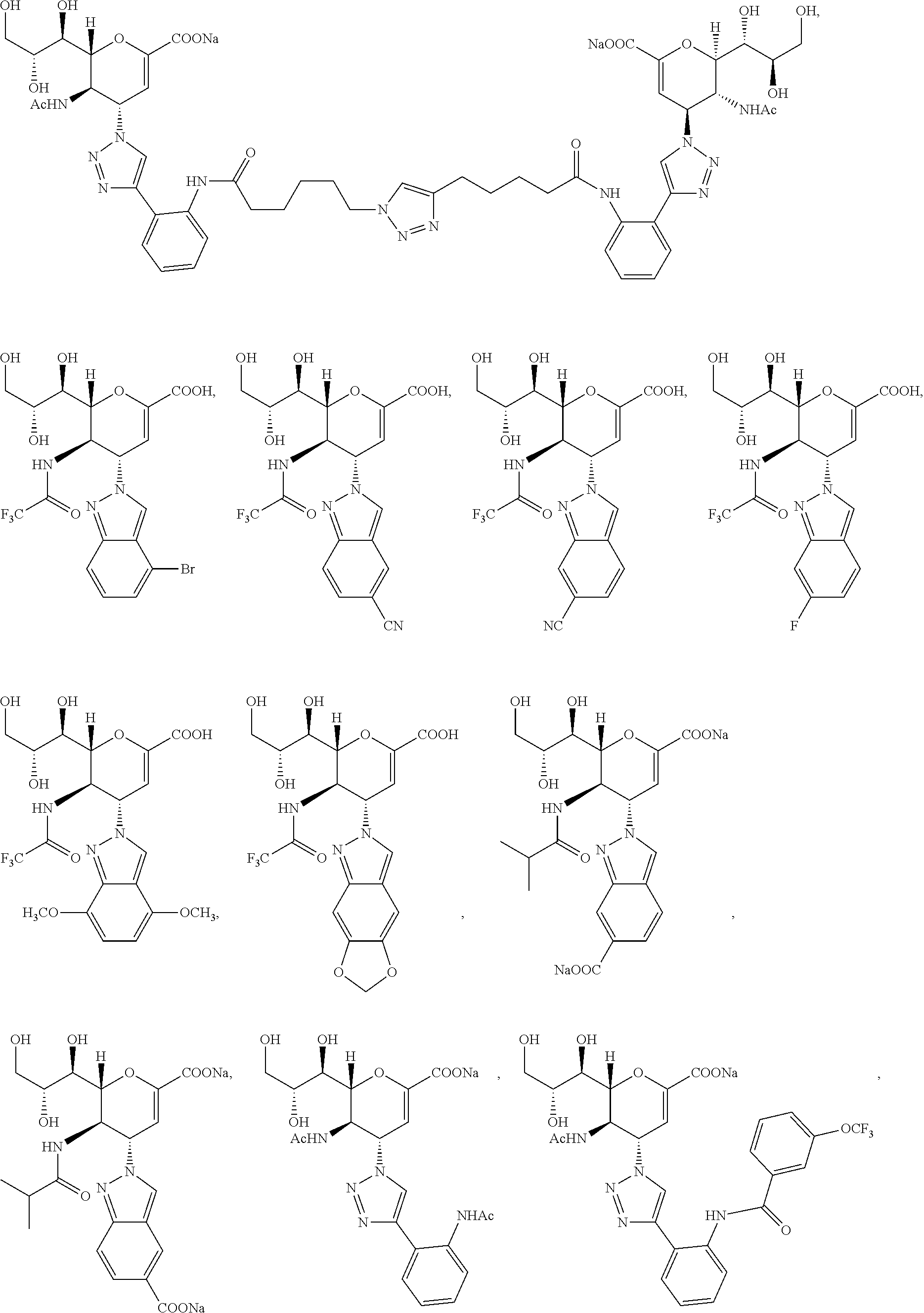 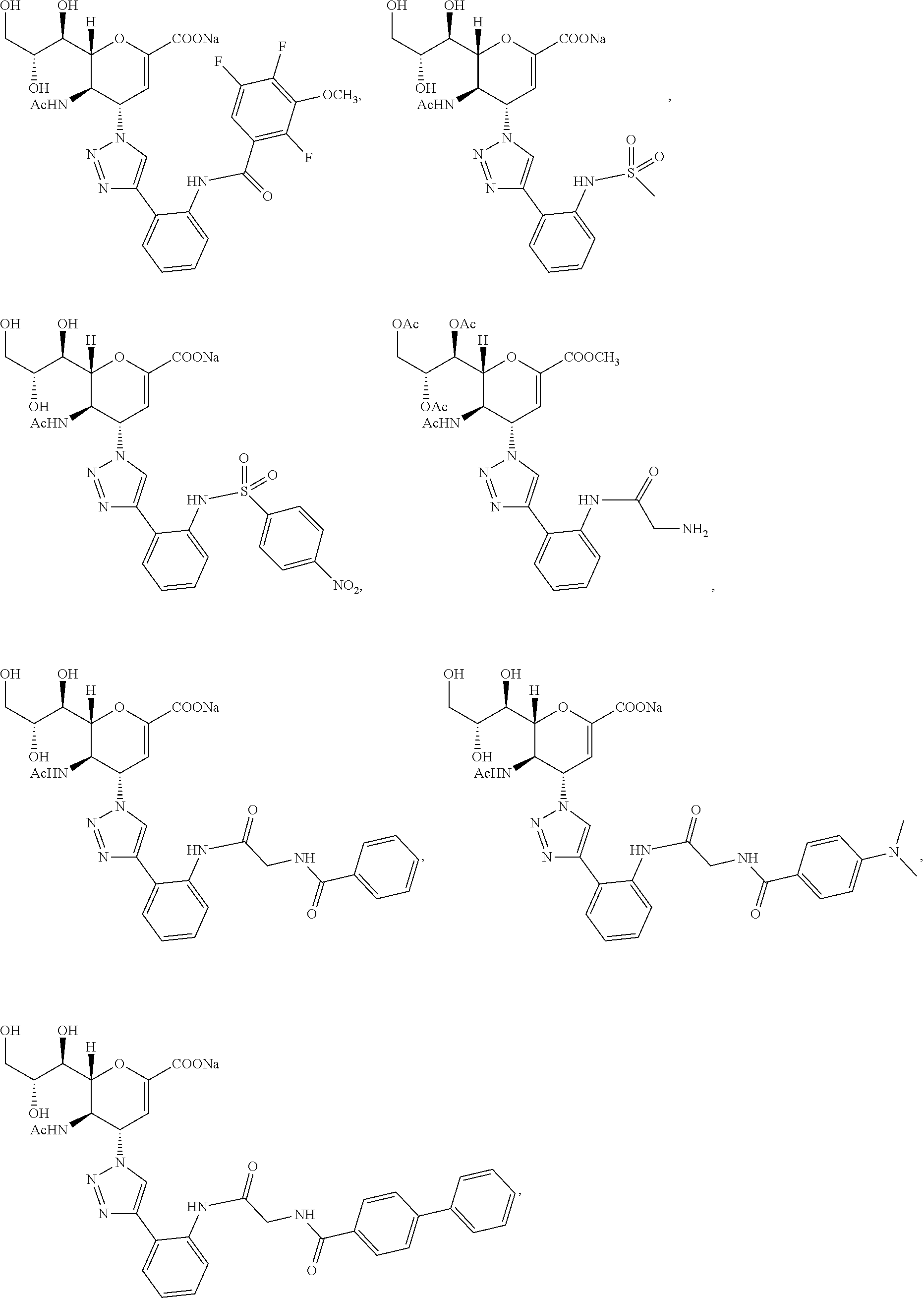 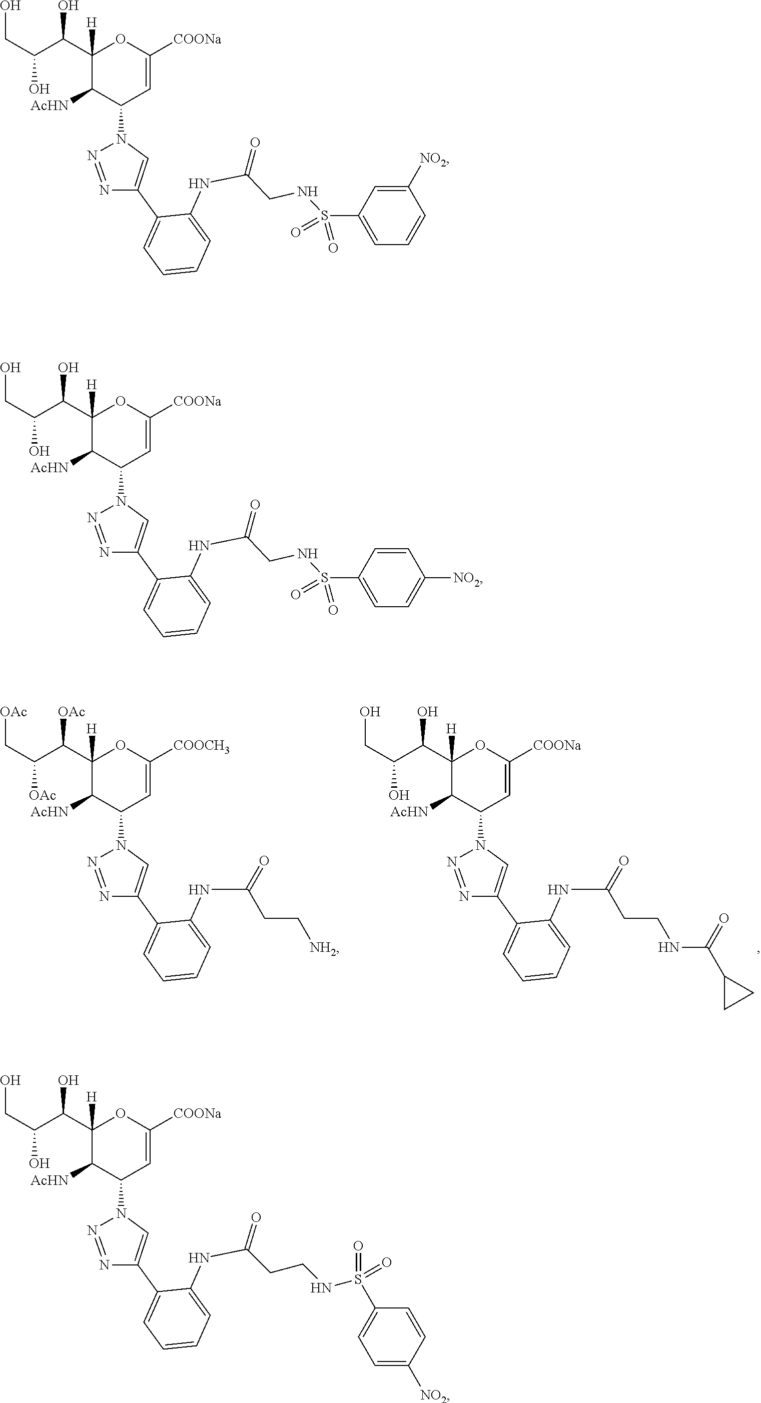 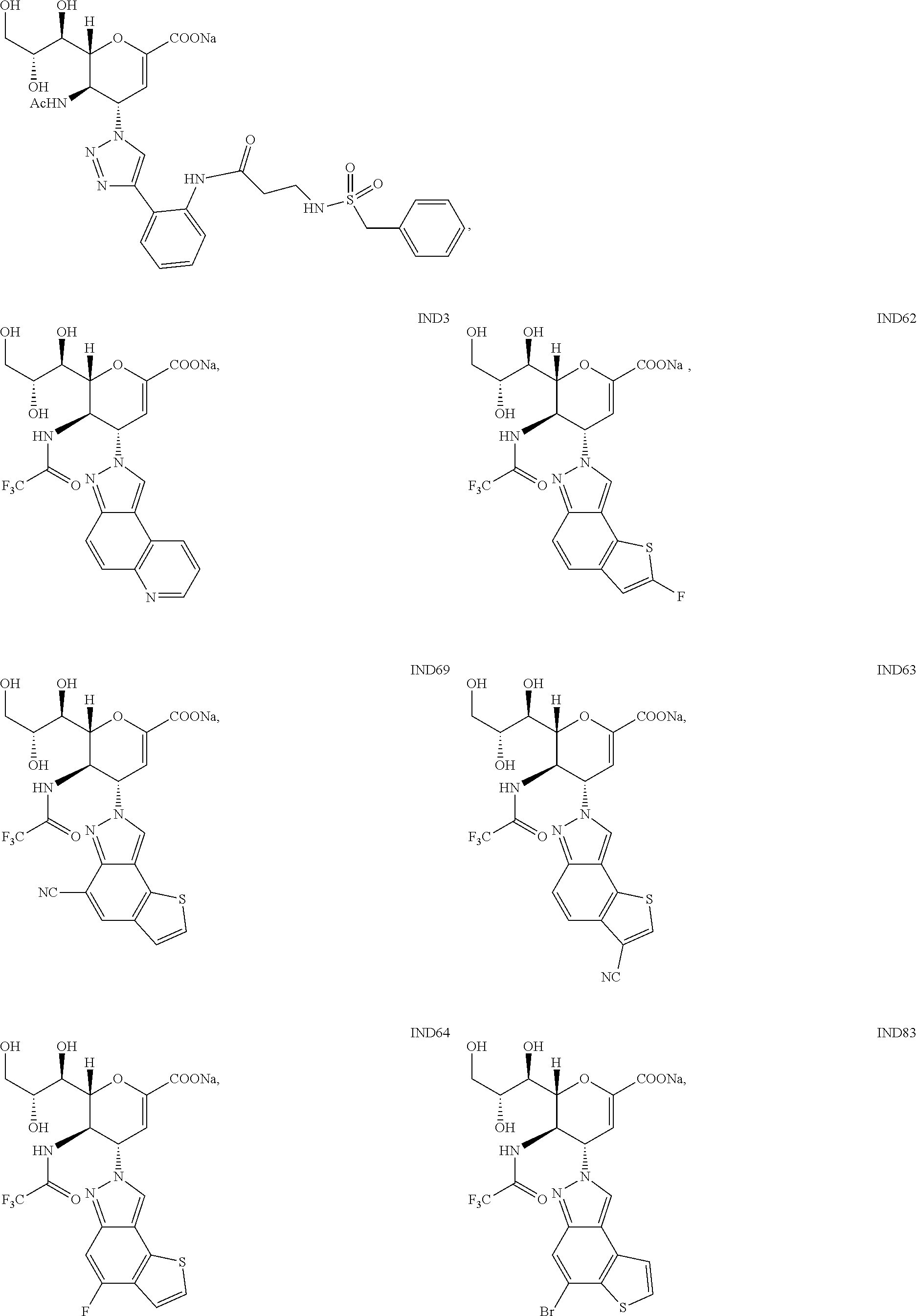 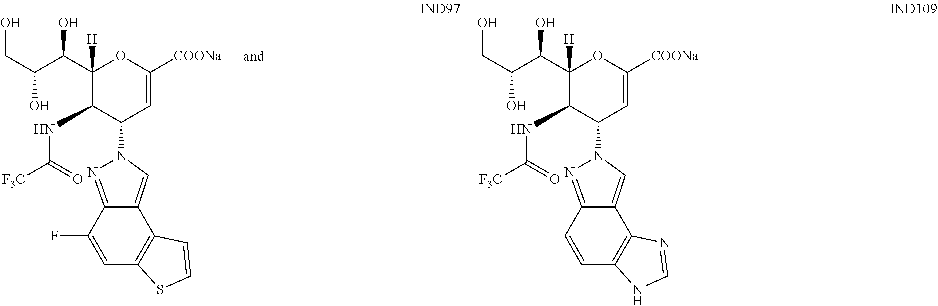
and protected forms thereof and analogues thereof wherein the C-2 carboxy group is in the protonated form, sodium salt form or prodrug form and wherein the R₄ position is substituted with any —NHC(O)R group wherein R is C₁-C₄ alkyl or haloalkyl.

16. A pharmaceutical composition comprising an effective amount of a compound of claim 1, or a pharmaceutically acceptable salt thereof, and a pharmaceutically acceptable carrier, diluent and/or excipient.

17. (canceled)

18. A method of treating a disease, disorder or condition caused by viral infection in a patient including the step of administering an effective amount of a compound of claim 1, or a pharmaceutically effective salt thereof, to the patient.

19. (canceled)

20. The method of claim 19 wherein the infection is caused by a virus selected from the group consisting of influenza A virus, influenza B virus, influenza C virus, influenza D virus, parainfluenza virus, respiratory syncytial virus (RSV) and human metapneumovirus (hMPV).

21.-22. (canceled)

23. A method of modulating viral haemagglutinin and/or neuraminidase function including the step of contacting the viral haemagglutinin-neuraminidase with a compound of claim 1, or a pharmaceutically effective salt thereof.

24. The method of claim 23 wherein the modulating is inhibiting and the viral haemagglutinin-neuraminidase is a parainfluenza haemagglutinin-neuraminidase.
